(19) United States
(12) Patent Application Publication
(10) Pub. No.: US 2005/0048574 A1
(43) Pub. Date: Mar. 3, 2005

(54) Biomarkers for diagnosing rheumatoid arthritis

(76) Inventors: Aaron B. Kantor, San Carlos, CA (US); Christopher Becker, Palo Alto, CA (US); Howard Schulman, Palo Alto, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/801,990

(22) Filed: Mar. 15, 2004

Related U.S. Application Data
(60) Provisional application No. 60/455,037, filed on Mar. 14, 2003.

(51) Int. Cl. G01N 33/53

(52) U.S. Cl. 435/7.1

(57) ABSTRACT

Biological markers for rheumatoid arthritis (RA) are disclosed. Also disclosed are the uses of such markers to diagnose and treat RA, monitor progression of the disease, evaluate therapeutic interventions, and screen candidate drugs in a clinical or preclinical trial.

### CROSS-REFERENCE TO RELATED APPLICATIONS

This application claims priority under 35 U.S.C. § 119 from U.S. Application Ser. No. 60/455,037, filed Mar. 14, 2003, which is incorporated herein in its entirety by reference.

### FIELD OF THE INVENTION

The present invention relates to biological markers for rheumatoid arthritis (RA). More specifically, the present invention relates to the use of such markers to diagnose and treat RA, monitor progression of the disease, evaluate therapeutic interventions, and screen candidate drugs in a clinical or preclinical trial.

### BACKGROUND OF INVENTION

Rheumatoid arthritis (RA) is a chronic inflammatory disorder of the small joints that also has pronounced and potential disabling systemic consequences, including fatigue, malaise and fever. It is estimated that about 2.1 million people in the United States have RA. The disease typically begins in middle age and occurs with increased frequency in older people. For reasons that are not fully understood, about two to three times as many women as men have the disease.

Although the etiology of the disease is unknown, its pathology evolves with common characteristics over time. The inflamed joint is characterized by synovial fibroblast hyperplasia, infiltration of activated lymphocytes and macrophages, and high levels of neutrophils. Early events are believed to include an inflammatory response initiated by unknown mediators. Activated CD4 T-cells appear to amplify and perpetuate the inflammation. The presence of activated T-cells can induce polyclonal B-cell activation.

Tissue damage inevitably progresses, releasing autoantigens, and the extent of the T-cell response broadens. Eventually, the constant inflammatory environment may lead to transformation of the synovial fibroblasts, yielding destructive potential that is independent of T-cells and macrophages. The pro-inflammatory cytokines such as TNF-α, produced mainly by macrophages in the joint, and the cytokines they induce such as IL-6 are systemically active, present in the serum and augment hepatic synthesis of acute-phase proteins. These cytokines are potent stimulators of mesenchymal cells, such as synovial fibroblasts, osteoclasts and chondrocytes, which release tissue-destroying matrix metalloproteinases which ultimately lead to the erosion of bone and cartilage.

The diagnosis of RA is typically made based on medical history, physical examination and X-ray imaging of the affected joint(s). Antibodies directed to the crystallizable fragment of IgG molecules (rheumatoid factor) are often found in high levels in RA. However, not everyone who has RA tests positive for rheumatoid factor and some who test positive never develop the disease. Neutrophils, for example, are generally elevated in RA, while CD8 T-cells are generally reduced. Also, the CD4:CD8 T-cell ratio is higher in RA subjects. Cush & Lipsky, Arthritis Rheum., 31:1230-8 (1988); Dale, Neutropenia and Neutophilia, in WILLIAMS HEMATOLOGY, Beutler et al., eds., McGraw Hill: New York. p. 823-834 (2001). Other factors associated with RA include, for example, C-reactive protein and antibodies to citrulline-containing peptides. However, there is no consensus panel of RA-specific markers. Early diagnosis and knowledge of disease progression would allow early initiation of treatment when it is most appropriate and potentially would be of the greatest benefit to the patient.

A number of approaches are used to treat RA. Nonsterioidal anti-inflammatory drugs (NSAIDS) are typically used to reduce pain, swelling and inflammation. Disease-modifying anti-rheumatic drugs (DMARDS) are used to slow progression of the disease and to prevent further joint injury (e.g., gold salts, antimalarials, methotrexate, Penicillamine, Sulfazalazine). The mechanism of action for these drugs is not fully understood. Biologic response modifiers differ from traditional DMARDS in that they target specific constituents of the immune system that contribute to the disease, while leaving other constituents of the immune system intact. This includes anti-TNF alpha inhibitors. While some patients respond well to a particular DMARD or combination of DMARDs, others show only modest benefit or no significant improvement. Furthermore, these drugs are associated with a number of serious side effects. The search for better therapeutics with fewer side effects is a subject of active research.

Therefore, there is a need to identify biochemical markers for RA. There is also a need for improved compositions and methods for diagnosing RA, and improved compositions and methods for treating RA.

### SUMMARY OF THE INVENTION

One aspect of the invention provides polypeptides that have been identified as differentially expressed in biological samples obtained from RA subjects as compared to samples obtained from non-RA subjects (“polypeptide markers”). The invention also provides polypeptides that have substantial homology with polypeptide markers, modified polypeptide markers, and fragments of polypeptide markers. The invention also includes precursors and successors of the polypeptide markers in biological pathways. The invention also provides molecules that comprise a polypeptide marker, a polypeptide that has substantial homology with a polypeptide marker, a modified polypeptide marker, a fragment of a polypeptide marker, or a precursor or successor of a polypeptide marker (e.g., a fusion protein). As used herein, the term “polypeptides of the invention” shall be understood to refer to any or all of the foregoing polypeptides.

Another aspect of the invention provides polynucleotides encoding polypeptides of the invention (“polynucleotide markers”). The invention also provides polynucleotides that have substantial homology with polynucleotide markers, modified polynucleotide markers, and fragments of polynucleotide markers. The invention also provides molecules that comprise a polynucleotide marker, a polynucleotide that has substantial homology with a polynucleotide marker, a modified polynucleotide marker or a fragment of a polynucleotide marker (e.g., a vector). Because of the redundancy (degeneracy) of the genetic code, a number of polynucleotides markers are capable of encoding a single polypeptide of the invention. As used herein, the term “polynucleotides of the invention” shall be understood to refer to any or all of the foregoing polynucleotides.

Another aspect of the invention provides cell populations that have been identified as differentially expressed in biological samples obtained from RA subjects as compared to samples obtained from non-RA subjects. As used herein, the terms “cell populations of the invention” or “cell population markers” shall be understood to refer to any or all of such cell populations.

Another aspect of the invention provides antibodies that selectively bind to a polypeptide of the invention, polynucleotide of the invention, or a cell population of the invention (e.g., a molecule associated with a cell that is a member of a cell population). The invention also provides methods for producing an antibody that selectively binds to a polypeptide of the invention, polynucleotide of the invention, or cell population of the invention.

Another aspect of the invention provides compositions comprising (i) a polypeptide of the invention, (ii) a polynucleotide of the invention, (iii) an antibody against a polypeptide of the invention, polynucleotide of the invention or cell population of the invention, (iv) an inhibitor of the activity of a polypeptide of the invention, a polynucleotide of the invention or a cell population of the invention, or (v) a molecule that can increase or decrease the level or activity of a polypeptide of the invention, a polynucleotide of the invention or a cell population of the invention. Such compositions may be pharmaceutical compositions formulated for use as therapeutics.

Another aspect of the invention provides a method for detecting the level or activity of a polypeptide of the invention, a polynucleotide of the invention or a cell population of the invention. In one embodiment, for example, the method comprises contacting an antibody that selectively binds to a polypeptide of the invention with a biological sample suspected of containing such polypeptide under conditions that would permit the formation of a stable complex and detecting any stable complexes that are formed. In another embodiment, the method comprises determining the activity of a polypeptide of the invention that functions as an enzyme. In another embodiment, the method comprises determining the level of a polynucleotide of the invention in a cell obtained from the subject.

Another aspect of the invention provides a method for diagnosing RA in a subject by detecting the level or activity of a polypeptide of the invention, a polynucleotide of the invention, or a cell population of the invention in a biological sample obtained from the subject. For example, in one embodiment, the method comprises obtaining a biological sample from a subject suspected of having RA, or at risk for developing RA, and comparing the level of a polypeptide of the invention in the biological sample with the level or activity in a biological sample obtained from a non-RA subject or with a standard value or reference range. In some embodiments, the method is used for staging or stratifying subjects with RA, monitoring progression of the disease, response to therapy, or susceptibility to RA. In some embodiments, a plurality of polypeptides of the invention, polynucleotides of the invention, or cell populations of the invention are detected. In some embodiments, such plurality of polypeptides of the invention, polynucleotides of the invention, or cell populations, are detected in a pattern (e.g., two specific polypeptide markers are elevated and one specific cell population is decreased). In some embodiments, the method comprises detecting known markers of RA or considering other clinical indicia of RA in addition to detecting one or more polypeptides of the invention, polynucleotides of the invention or cell populations of the invention. Another aspect of the invention provides methods for monitoring therapeutic treatment of RA.

Another aspect of the invention provides methods for treating RA by administering to a subject a therapeutic agent that results in an increase or decrease in the level or activity of a polypeptide of the invention, a polynucleotide of the invention or a cell population of the invention (e.g., the level of a certain polypeptide marker in a sample obtained from the subject). In one embodiment, the therapeutic agent administered to the subject is one or more markers of the invention. For polypeptides of the invention, polynucleotides of the invention, or cell populations of the invention that are increased in biological samples obtained from RA subjects, the method comprises administering a therapeutic agent that decreases the level or activity of the polypeptide, polynucleotide or cell population. For polypeptides of the invention, polynucleotides of the invention, or cell populations of the invention that are decreased in biological samples obtained from RA subjects, the method comprises administering a therapeutic agent that increases the level or activity of the polypeptide, polynucleotide, or cell population.

Another aspect of the invention provides a method for screening a candidate compound for use as a therapeutic agent for treating RA. In one embodiment, the method comprises administering the candidate compound to an RA subject and screening for the ability to increase or decrease the level or activity of a polypeptide of the invention, a polynucleotide of the invention, or a cell population of the invention in a biological sample obtained from the subject.

Another aspect of the invention provides a kit for performing one or more of the methods described above. In another embodiment, the kit is for detecting the level or activity of a polypeptide of the invention, a polynucleotide of the invention, or a cell population of the invention and includes an antibody that selectively binds to the polypeptide, polynucleotide or cell population.

Other features and advantages of the invention will become apparent to one of skill in the art from the following description and claims.

### DETAILED DESCRIPTION OF THE INVENTION

The present inventors have discovered polypeptides, polynucleotides, and cell populations that are differentially expressed in biological samples obtained from RA subjects compared to samples obtained from non-RA subjects. The levels and activities of these polypeptides, polynucleotides, and cell populations can be used as biological markers indicative of rheumatoid arthritis (RA).

According to one definition, a biological marker is “a characteristic that is objectively measured and evaluated as an indicator of normal biologic processes, pathogenic processes, or pharmacological responses to therapeutic interventions.” NIH Biomarker Definitions Working Group (1998). Biological markers can also include patterns or ensembles of characteristics indicative of particular biological processes (“panel of markers”). The marker measurement can be increased or decreased to indicate a particular biological event or process. In addition, if a marker measurement typically changes in the absence of a particular biological process, a constant measurement can indicate occurrence of that process.

Marker measurements may be of the absolute values (e.g., the molar concentration of a molecule in a biological sample) or relative values (e.g., the relative concentration of two molecules in a biological sample). The quotient or product of two or more measurements also may be used as a marker. For example, some physicians use the total blood cholesterol as a marker of the risk of developing coronary artery disease, while others use the ratio of total cholesterol to HDL cholesterol. See discussion of marker measurement and discovery in Ringold et al., “Phenotype and Biological Marker Identification System” WO 00/65472 (published Nov. 2, 2000), incorporated herein by reference in its entirety.

In the invention, the markers are primarily used for diagnostic purposes. However they may also be used for therapeutic, drug screening and patient stratification purposes (e.g., to group patients into a number of “subsets” for evaluation), as well as other purposes described herein, including evaluation the effectiveness of an RA therapeutic.

The practice of the invention employs, unless otherwise indicated, conventional methods of analytical biochemistry, microbiology, molecular biology and recombinant DNA generally known techniques within the skill of the art. Such techniques are explained fully in the literature. (See, e.g., Sambrook et al. Molecular Cloning: A Laboratory Manual. 3rd, ed., Cold Spring Harbor Laboratory, Cold Spring Harbor Laboratory Press, Cold Spring Harbor, N.Y., 2000; DNA Cloning: A Practical Approach, Vol. I & II (Glover, ed.); Oligonucleotide Synthesis (Gait, ed., Current Edition); Nucleic Acid Hybridization (Hames & Higgins, eds., Current Edition); Transcription and Translation (Hames & Higgins, eds., Current Edition); CRC Handbook of Parvoviruses, Vol. I & II (Tijessen, ed.); Fundamental Virology, 2nd Edition, Vol. I & II (Fields and Knipe, eds.)).

The terminology used herein is for describing particular embodiments and is not intended to be limiting. As used herein, the singular forms “a,” “and” and “the” include plural referents unless the content and context clearly dictate otherwise. Thus, for example, a reference to “a marker” includes a combination of two or more such markers.

Unless defined otherwise, all scientific and technical terms are to be understood as having the same meaning as commonly used in the art to which they pertain. For the purposes of the invention, the following terms are defined below.

I. Definitions

As used herein, the term “antibody” refers to any molecule that reversibly binds to another with the required selectivity. Thus, the term includes any molecule that is capable of selectively binding to a marker of the invention. The term includes an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required selectivity (see “selectively binding” defined, infra). The term also includes non-immunoglobin species. Thus, for example, a binding molecule may be a member of a binding pair such as enzyme with respect to a substrate, substrate with respect to an enzyme, lectin with respect to a carbohydrate, carbohydrate with respect to a lectin, receptor with respect to a hormone, hormone with respect to a receptor, ligand with respect to a counterligand, counterligand with respect to a ligand, aptamer with respect to its target, target with respect to its aptamer, and so on. Consistent with the foregoing, an “antibody” described as selectively binding to a polypeptide of the invention should be understood as including any molecule that reversibly binds to the polypeptide with the required selectivity.

As used herein, the term “biological sample” means any biological substance, including but not limited to blood (including whole blood, leukocytes prepared by lysis of red blood cells, peripheral blood mononuclear cells, plasma and serum), sputum, urine, semen, cerebrospinal fluid, bronchial aspirate, sweat, feces, synovial fluid, cells, and whole or manipulated tissue.

As used herein, the term “cell population” means a set of cells having characteristics in common. The characteristics include without limitation the presence and level of one, two, three or more cell-associated molecules (e.g., cell-surface antigens). One, two, three or more cell-associated molecules can thus define a cell population.

As used herein, the term “cell-associated molecule” means any molecule associated with a cell. This includes without limitation (i) intrinsic cell surface molecules such as proteins, glycoproteins, lipids, and glycolipids; (ii) extrinsic cell surface molecules such as cytokines bound to their receptors, immunoglobulin bound to Fc receptors, foreign antigen bound to B-cell or T-cell receptors and auto-antibodies bound to self antigens; (iii) intrinsic internal molecules such as cytoplasmic proteins, carbohydrates, lipids and mRNA, and nuclear protein and DNA (e.g., genomic and somatic nucleic acids); and (iv) extrinsic internal molecules such as viral proteins and nucleic acid. As an example, there are hundreds of leukocyte cell surface proteins or antigens, including leukocyte differentiation antigens (e.g., CD antigens), antigen receptors (e.g., B-cell receptor and T-cell receptor) and major histocompatibility complexes. Each of these classes encompasses a vast number of proteins.

As used herein, the term “differentially expressed” refers to the level or activity of a constituent in a first sample (or set of samples) as compared to the level or activity of the constituent in a second sample (or set of samples), where the method used for detecting the constituent provides a different level or activity when applied to the two samples (or sets of samples). Thus, for example, a polypeptide of the invention that is measured at one concentration in a first sample, and at a different concentration in a second sample is differentially expressed in the first sample as compared with the second sample. A marker would be referred to as “increased” in the first sample if the method for detecting the marker indicates that the level or activity of the marker is higher or greater in the first sample than in the second sample (or if the marker is detectable in the first sample but not in the second sample). Conversely, the marker would be referred to as “decreased” in the first sample if the method for detecting the marker indicates that the level or activity of the marker is lower in the first sample than in the second sample (or if the marker is detectable in the second sample but not in the first sample). In particular, a marker is referred to as “increased” or “decreased” in a sample (or set of samples) obtained from a subject (e.g., an RA subject, a subject suspected of having RA, a subject at risk of developing RA) if the level or activity of the marker is higher or lower, respectively, compared to the level of the marker in a sample (or set of samples) obtained from another subject (e.g., a non-RA subject) or subjects or a reference value or range.

As used herein, the terms “fold increase” and “fold decrease” refer to the relative increase or decrease in the level or activity of a marker in one sample (or set of samples) compared to another sample (or set of samples). A positive fold change indicates an increase in the level of a marker while a negative fold change indicates a decrease in the level of a marker. The increase or decrease may be measured by any method or technique known to those of skill in the art. As will be appreciated by one of skill in the art, the observed increase or decrease may vary depending on the particular method or technique that is used to make the measurement.

As used herein, the term “fragment” as applied to a polypeptide (e.g., “a fragment of a polypeptide”) refers to a single amino acid of a full-length polypeptide from which it has been derived or to a polymer of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the full-length polypeptide from which it has been derived. As used herein, the term “fragment” as applied to a polynucleotide (e.g., “a fragment of a polynucleotide”) refers to a single nucleic acid of a full-length polynucleotide or to a polymer of nucleic acid residues comprising a nucleic acid sequence that has at least 15 contiguous nucleic acid residues, at least 30 contiguous nucleic acid residues, at least 60 contiguous nucleic acid residues of a sequence of a full-length polynucleotide from which it has been derived.

As used herein, the term “isolated” as applied to a molecule or cell refers to a molecule or cell that has been removed from its natural environment. For example, a polypeptide can be considered isolated if it is separated from one or more metabolites, polynucleotides and other polypeptides with which it is naturally associated. Isolated molecules can be either prepared synthetically or purified from their natural environment (e.g., biological sample obtained from a subject). Standard methodologies known in the art can be employed to obtain and isolate the polynucleotides, polypeptides, antibodies, other molecules, and cells of the invention. The term “isolated” does not necessarily reflect the extent to which the molecule or cell has been purified.

As used herein, the term “marker” includes polypeptide markers, polynucleotide markers, and cell population markers. For clarity of disclosure, aspects of the invention will be described with respect to “polypeptide markers,” “polynucleotide markers” and “cell population markers.” However, statements made herein with respect to “polypeptide markers” are intended to apply to other polypeptides of the invention. Likewise, statements made herein with respect to “polynucleotide markers” are intended to apply to other polynucleotides of the invention. Thus, for example, a polynucleotide described as encoding a “polypeptide marker” is intended to encompass a polynucleotide that encodes a polypeptide marker, a polypeptide that has substantial homology to a polypeptide marker, a modified polypeptide marker, a fragment, precursor or successor of a polypeptide marker, and molecules that comprise a polypeptide marker, homologous polypeptide, a modified polypeptide marker or a fragment, precursor or successor of a polypeptide marker. Furthermore, consistent with their definition, supra, as sets of cells having characteristics in common, statements made herein with respect to “cell population markers (or “cell populations of the invention”) are intended also to apply to one or more cells that are members of the cell populations. Thus, for example, an antibody described as selectively binding to a “cell population of the invention” should be understood as including an antibody that selectively binds to a cell that is a member of the cell population.

As used herein, the phrase “capable of performing the function of that polypeptide in a functional assay” means that the polypeptide has at least 50% of the activity, at least 60% of the activity, at least 70% of the activity, at least 80% of the activity, at least 90% of the activity, or at least 95% of the activity of the polypeptide in the functional assay.

As used herein, the term “polypeptide” refers to a single amino acid or a polymer of amino acid residues of any length. A polypeptide includes without limitation an amino acid, an oligopeptide, a peptide and a protein. A polypeptide may be composed of a single polypeptide chain or two or more polypeptide chains. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention (e.g., formation of a disulfide bond, glycosylation, lipidation, methylation, acetylation, phosphorylation, conjugation with a labeling molecule).

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. Analogs of both the purine and pyrimidine base can differ from a corresponding naturally occurring moiety by having new substituent groups attached thereto, for example, 2,6-diaminopurine or didehydroribose, by having naturally occurring substituent groups deleted therefrom, or by having atoms normally present replaced by others, for example, 8-azaguanine. Polynucleotides can also comprise modified backbones, including, but not limited to, methyl phosponates, phosphorothioates, phosphordithioates, and PNA backbones. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention, including by in vitro manipulation). For every single-stranded polynucleotide of the invention, the invention also includes the complementary polynucleotide.

In some embodiments, a polypeptide marker or a polynucleotide marker is part of one or more biological pathways (e.g., amino acid metabolism, the urea cycle, the citric acid cycle, pentose phosphate pathway, glycogen synthesis and degradation pathways, fatty acid synthesis and breakdown pathways, prostaglandin and steroid biosynthesis, purine and pyrimidine synthesis, deoxyribonucleotide synthesis). The identification of such biological pathways and their members is within the skill of one in the art. Once a polypeptide of the invention or polynucleotide of the invention is identified as part of one or more biological pathways, the invention includes additional members of the pathway that precede or follow the polypeptide or polynucleotide by one step, two steps, three steps, or more steps. As used herein, the term “precursor” or “metabolic precursor” refers to a molecule (or reactant) that precedes the marker in the pathway while the term “successor” or “metabolic successor” refers to a molecule (or product) that follows the marker in the pathway.

As used herein, the terms “RA subject” and “a subject who has RA” refer to a subject who has been diagnosed with RA. The terms “non-RA subject” and “a subject who does not have RA” are refer to a subject who has not been diagnosed as having RA. Non-RA subjects may be healthy and have no other disease, or they may have a disease other than RA. While human subjects are described herein, it is to be understood that in some embodiments, subject refers to a laboratory animal.

As used herein, the term “selectively binding,” refers to the ability of antibodies to preferentially bind to an antigen (i.e., to be able to distinguish that antigen from unrelated constituents in a mixture). The antigen may be free of other constituents or part of a complex, such as associated with a cell. Binding affinities, commonly expressed as equilibrium association constants, typically range from about 10³ M⁻¹ to about 10¹² M⁻¹. Binding can be measured using a variety of methods known to those skilled in the art including immunoblot assays, immunoprecipitation assays, radioimmunoassays, enzyme immunoassays (e.g., ELISA), immunofluorescent antibody assays and immunoelectron microscopy. See, e.g., Sambrook et al., supra.

As used herein, the term “stringent hybridization conditions” refers to standard hybridization conditions under which polynucleotides are used to identify molecules having similar nucleic acid sequences. Such standard conditions are disclosed, for, example, in Sambrook et al., supra. Stringent hybridization conditions typically permit isolation of polynucleotides having at least 70% nucleic acid sequence identity, at least 80% nucleic acid sequence identity, at least 90% nucleic acid sequence identity, at least 95% nucleic acid sequence identity or at least 99% nucleic acid sequence identity with the polynucleotide being used to probe in the hybridization reaction. Formulae to calculate the appropriate hybridization and wash conditions to achieve hybridization permitting 30% or fewer mismatches of nucleotides are disclosed, for example, in Meinkoth et al., Anal. Biochem. 138:267-284 (1984), incorporated herein by reference in its entirety.

As used herein, the term “substantially homologous” (or “substantial homology” or a “homolog”) as applied to two or more polypeptides means (i) that there is at least 70% homology, at least 80% homology, at least 90% homology, at least 95% homology or at least 99% homology between their amino acid sequences, or (ii) that a polynucleotide encoding one of the polypeptides is capable of forming a stable duplex with the complementary sequence of a polynucleotide encoding the other polypeptide. As used herein, the term “substantially homologous” (or “substantial homology” or a “homolog”) as applied to two or more polynucleotides means (i) that there is at least 70% homology, at least 80% homology, at least 90% homology, at least 95% homology or at least 99% homology between their amino acid sequences, or (ii) that one or more strands of one of the polynucleotides are capable of forming a stable duplex with one or more strands of the other.

II. Polypeptide and Metabolite Markers

One embodiment of the invention is based, in part, on the discovery that certain polypeptide markers are differentially expressed in biological samples obtained from RA subjects compared to biological samples obtained from non-RA subjects and, in particular, that such differences are statistically significant.

A high molecular weight fraction, containing proteins with molecular weights greater than about 5-kDa, was separated from serum samples, individually, obtained from RA subjects and serum samples obtained from non-RA subjects. After removal of high abundance proteins, the high molecular weight fraction was digested with trypsin. The high molecular weight fraction was then separated by chromatographic means and analyzed by mass spectrometry. The resulting spectra were compared to identify peaks that were associated with markers differentially expressed in subjects with RA. In some cases, peaks associated with markers differentially expressed in subjects with RA were further investigated to identify the polypeptide markers represented by the peak. Wang et al., Anal. Chem., 75:4818-4826 (2003).

Table 1 lists the full-length proteins for which a plurality of fragments were identified as differentially expressed (significantly increased) in serum samples obtained from RA subjects compared with serum samples obtained from non-RA subjects.

Table 2 lists the full-length proteins for which a plurality of fragments were identified as differentially expressed (significantly decreased) in serum samples obtained from RA subjects compared with serum samples obtained from non-RA subjects.

Table 3 lists polypeptides that were identified as differentially expressed (significantly increased) in serum samples obtained from RA subjects compared with serum samples obtained from non-RA subjects.

Table 4 lists polypeptides that were identified as differentially expressed (significantly decreased) in serum samples obtained from RA subjects compared with serum samples obtained from non-RA subjects.

Table 5 lists additional polypeptides that were identified as differentially expressed (significantly increased) in serum samples obtained from RA subjects compared with serum samples obtained from non-RA subjects.

Table 6 lists additional polypeptides that were identified as differentially expressed (significantly decreased) in serum samples obtained from RA subjects compared with serum samples obtained from non-RA subjects.

The polypeptide markers of the invention that are set forth in Table 1, Table 2, Table 3, Table 4, Table 5 and Table 6 are each described by (i) the mass to charge ratio (m/z), (ii) the chromatographic retention time (R.T.), (iii) the charge state of a molecular ion (z), (iv) the protonated parent mass (M+H), (v) the expression ratio (exp. ratio), which is a ratio of mean group intensities indicating the relative normalized signal for RA subject group compared to non-RA subject group, (vi) fold change, and (v) the applicable p-value range. The polypeptide markers set forth in Table 1, Table 2, Table 5 and Table 6 are also described by their corresponding identification number from NCBI's reference sequence database (Accession # and gi #) and additional identifying information (e.g., the name or sequence of the peptide marker as contained in the NCBI queried database and database searching using the TurboSEQUEST and Mascot software programs). As one of skill in the art will appreciate, the physical and chemical properties presented in the Tables is sufficient to distinguish the polypeptides from other materials; in particular, the polypeptides are uniquely identified by M+H value, as well as the m/z value and R.T. values within the given experimental platform (see Examples).

Some variation is inherent in the measurements of physical and chemical characteristics of the markers. The magnitude of the variation depends to some extent on the reproducibility of the separation means and the specificity and sensitivity of the detection means used to make the measurement. Preferably, the method and technique used to measure the markers is sensitive and reproducible. The m/z and R.T. values may vary to some extent depending on a number of factors relating to the protocol used for the chromatography and the mass spectrometry parameters (e.g., solvent composition, flow rate). As one of skill in the art will appreciate, the data set forth in the Tables (e.g., M+H values) reflects to some extent the equipment and conditions used to make the measurements. The values stated in the Tables were obtained using the equipment and conditions described in the Examples. When a sample is processed and analyzed in this manner, the retention time of a marker is about the value stated for the marker and the marker has a mass-to-charge ratio of about the value stated for the marker.

The polypeptide markers of the invention are useful in methods for diagnosing RA, determining the extent and/or severity of the disease, monitoring the progression of the disease and/or response to therapy. The markers are also useful in methods for treating RA and for evaluating the efficacy of treatment. The markers may be targets for treatment. The markers may also be used as pharmaceutical compositions or in kits. The markers may also be used to screen candidate compounds that modulate the level or activity of the markers. The markers may also be used to screen candidate drugs for their ability to treat RA.

In one embodiment, the invention provides a polypeptide marker described in Table 1, Table 2, Table 3, Table 4, Table 5 or Table 6. In another embodiment, the invention provides a molecule that comprises such a polypeptide marker.

In another embodiment, the invention provides a polypeptide that is substantially homologous to a polypeptide marker described in Table 1, Table 2, Table 3, Table 4, Table 5 or Table 6. In another embodiment, the invention provides a molecule that comprises such a polypeptide.

In another embodiment, the invention provides a polypeptide having an M+H value of about the value stated for a polypeptide marker described in Table 1, Table 2, Table 3, Table 4, Table 5 or Table 6. In another embodiment, the invention provides a molecule that comprises such a polypeptide.

In another embodiment, the invention provides a polypeptide having an M+H value within 1.0% (more particularly within 0.5%, more particularly within 0.1%, more particularly, within 0.05%, more particularly within 0.01%) of the M+H value stated for a polypeptide marker described in Table 1, Table 2, Table 3, Table 4, Table 5 or Table 6. In another embodiment, the invention provides a molecule that comprises such a polypeptide.

In another embodiment, the invention provides a polypeptide that is a fragment, precursor, successor or modified version of a polypeptide marker described in Table 1, Table 2, Table 3, Table 4, Table 5 or Table 6. In another embodiment, the invention provides a molecule that comprises such a polypeptide.

In another embodiment, the invention provides a polypeptide that is structurally different from a polypeptide marker described in Table 1, Table 2, Table 3, Table 4, Table 5 or Table 6 but is capable of performing the function of that polypeptide marker in a functional assay. For example, such a polypeptide may have amino acid sequence that is changed only in nonessential amino acid residues from a polypeptide marker described in Table 1, Table 2, Table 3, Table 4, Table 5 or Table 6. In another embodiment, the invention provides a molecule that comprises such a polypeptide.

Polypeptides of the invention may be isolated by any suitable method known in the art. Native polypeptide markers can be purified from natural sources by standard methods known in the art (e.g., chromatography, centrifugation, differential solubility, immunoassay). In one embodiment, polypeptide markers may be isolated from a serum sample using the chromatographic methods disclosed herein. In another embodiment, polypeptide markers may be isolated from a sample by contacting the sample with substrate-bound antibodies that selectively bind to the polypeptide marker. Alternatively, an isolated polypeptide marker can be produced using recombinant DNA technology or chemical synthesis.

An isolated polypeptide of the present invention can be produced in a variety of ways. Given the amino acid sequence or the corresponding DNA, cDNA, or mRNA that encodes them, polypeptides markers may be synthesized using recombinant or chemical methods. For example, polypeptide markers can be produced by transforming a host cell with a nucleotide sequence encoding the polypeptide marker and cultured under conditions suitable for expression and recovery of the encoded protein from the cell culture. See, e.g., Hunkapiller et al., Nature 310:105-111 (1984). Polypeptides of the present invention can be purified using a variety of standard protein purification techniques.

III. Polynucleotides Encoding Polypeptide Markers

In one aspect, the invention provides a polynucleotide that encodes the polypeptides of the invention. Such polynucleotides include without limitation genomic DNA, cDNA and mRNA transcripts.

In one embodiment, the invention provides a polynucleotide that encodes a polypeptide marker described in Table 1, Table 2, Table 3, Table 4, Table 5 or Table 6, or that encodes a molecule that comprises such a polypeptide marker.

In another embodiment, the invention provides a polynucleotide that encodes a polypeptide that is substantially homologous to a polypeptide marker described in Table 1, Table 2, Table 3, Table 4, Table 5 or Table 6, or that encodes a molecule that comprises such a polypeptide.

In another embodiment, the invention provides a polynucleotide that encodes a polypeptide having an M+H value of about the value stated for a polypeptide marker described in Table 1, Table 2, Table 3, Table 4, Table 5 or Table 6, or that encodes a molecule that comprises such a polypeptide.

In another embodiment, the invention provides a polynucleotide that encodes a polypeptide having an M+H value within 1% (more particularly within 0.5%, more particularly within 0.1%, more particularly, within 0.05%, more particularly within 0.01% of the M+H value stated for a polypeptide marker described in Table 1, Table 2, Table 3, Table 4, Table 5 and Table 6, or that encodes a molecule that comprises such a polypeptide.

In another embodiment, the invention provides a polynucleotide that encodes a polypeptide that is a fragment, precursor, successor or modified version of a polypeptide marker described in Table 1, Table 2, Table 3, Table 4, Table 5 and Table 6, or that encodes a molecule that comprises such a polypeptide.

In another embodiment, the invention provides a polynucleotide that encodes a polypeptide that is structurally different from a polypeptide marker described in Table 1, Table 2, Table 3, Table 4, Table 5 and Table 6 but is capable of performing the function of that polypeptide marker in a functional assay, or that encodes a molecule that comprises such a polypeptide.

In another embodiment, the invention provides a polynucleotide that is a fragment or modified version or is substantially homologous to any of the above-described polynucleotides.

Many of the polypeptides listed in Table 3, Table 4, Table 5 and Table 6 are fragments of full-length proteins, either because they were present as such in the serum sample or as a result of the trypsin digestion that was performed during the processing of the serum samples. In many cases, the sequence of the full-length protein can be ascertained from the amino acid sequence of the fragment by searching a protein sequence database. In any event, the full-length proteins comprising the fragments are included within the scope of the polypeptides of the invention.

Polynucleotides that encode polypeptides of the invention can be used to screen existing genomic, cDNA or expression libraries to find the gene that encodes the polynucleotide of the invention. A library is typically screened using a probe that is complementary either to (i) the polynucleotide that encodes a polypeptide of the invention or (ii) the complement of such polynucleotide. Hybridization is monitored by any suitable method known in the art. Once located, the gene that encodes a polynucleotide of the invention can be cloned. The protein product of such a gene is included within the scope of the polypeptides of the invention.

Alternatively, the sequence of the polynucleotide that encodes a polypeptide of the invention can be used to search public or private computer databases (e.g., SWISS-PROT, GenBank) that will provide the gene sequence (or gene sequences) comprising the polynucleotide sequence and/or the amino acid sequence of the gene product.

The polynucleotides of the invention can be used to synthesize the polypeptides of the invention. In addition, the polynucleotides of the invention may be measured instead of (or in addition to) the polypeptides of the invention in a method of the invention. Thus, for example, if the level of a polypeptide marker is increased in RA-subjects, an increase in the level of the mRNA that encodes the polypeptide marker may be used, rather than the level of the polypeptide marker (e.g., to diagnose RA in the subject). As one of skill in the art will recognize, however, the level of mRNA is typical not directly proproportional to the level of protein, even in a given cell. Furthermore, mRNA level will not indicate post-translational modifications of the protein.

Polynucleotide markers may be isolated by any suitable method known in the art. A native polynucleotide of the invention can be obtained from its natural source by standard methods known in the art (e.g., chromatography, centrifugation, differential solubility, immunoassay). In one embodiment, a polynucleotide marker may be isolated from a mixture by contacting the mixture with substrate bound probes that are complementary to the polynucleotide marker under hybridization conditions.

Alternatively, an isolated polynucleotide of the invention may be produced by any suitable chemical or recombinant method known in the art. In one embodiment, for example, a polypeptide marker can be produced using polymerase chain reaction (PCR) amplification. In another embodiment, a polynucleotide marker can be synthesized from appropriate reactants using the methods and techniques of organic chemistry.

IV. Cell Populations

One embodiment of the invention is based, in part, on the discovery that certain cell populations are differentially expressed in biological samples obtained from RA subjects compared to biological samples obtained from non-RA subjects and, in particular, that such differences are statistically significant.

A large number of cellular variables were analyzed, including cell counts, cell ratios, and the level of cell-associated molecules, using microvolume laser scanning cytometry (MLSC). Walton et al., Proc. SPIE-Int. Soc. Opt. Eng., 3926:192-201 (2000). Blood samples obtained from RA subjects and non-RA subjects were stained with fluorophore-labeled antibodies specific for cell surface antigens and loaded into optical-quality capillary arrays. Typically, three antibody reagents, each with a different fluorescent tag and each detected in a different channel, were used per assay. Each assay typically contained one or two antibodies to the major cell populations (neutrophils, eosinophils, monocytes, total T-cells, CD4 T-cells, B-cells and NK cells) and one or two antibodies to subsetting antigens that may indicate the functional state, activation state or adhesion characteristics of the population. The capillary was imaged and the fluorescent events were detected. Peaks corresponding to antibody-labeled cells were identified with image processing software. See, Norton et al. Prof. SPIE-Int. Soc. Opt. Eng., 3921:20-30 (2000), incorporated herein by reference in its entirety. Unlabeled cells (e.g., erythrocytes and leukocytes not expressing the target antibodies) were not identified. Compensation was made for spectral overlap of the dyes with respect to the intensity data, so result values were proportional to the amount of dye-antibody reagent on each cell. Because the volume of the scan is precisely defined, absolute cell counts (cells per μL of blood) were determined.

Table 7 lists the cell populations that were identified as differentially expressed (significantly increased) in serum samples obtained from RA subjects compared with serum samples obtained from non-RA subjects.

Table 8 lists cell populations that were identified as differentially expressed (significantly decreased) in serum samples obtained from RA subjects compared with serum samples obtained from non-RA subjects.

The cell population markers set forth in Table 7 and Table 8 are each described by (i) general cell type, (ii) assay, (iii) cell population, (iv) property (i.e., count, ratio, or relative antigen intensity); (v) p-value (either adjusted or univariate, as appropriate depending on the normality of the data), and (vi) the effect size (difference of means between the two groups divided by the weighted standard deviation) which indicates how well the groups are separated.

Some variation is inherent in the measurement of the levels of the cell population markers. The magnitude of the variation depends to some extent on the reproducibility of the sample preparation procedures and on the specificity and sensitivity of the detection means used to make the measurement. Preferably, the method and technique used to measure the cell population makers is sensitive and reproducible. As one of skill in the art will appreciate, the data set forth in Tables 7 and 8 reflects to some extent the equipment and conditions used to make the measurements. The values stated in the Tables were obtained using the equipment and conditions described in the Examples. When a sample is processed and analyzed in this manner, the values are about those stated for the marker (within about 10%, within about 5%, within about 1% of the value stated).

The cell population markers of the invention are useful in methods for diagnosing RA, determining the extent and/or severity of the disease, monitoring the progression of the disease and/or response to therapy. The markers are also useful in methods for evaluating the efficacy of treatment for RA. The cell population markers can also be used in kits. The cell population markers may also be used to screen candidate compounds that modulate the expression of the markers. The cell population markers may also be used to screen candidate drugs for their ability to treat RA.

V. Antibodies

In one aspect, the invention provides antibodies that selectively bind to a polypeptide of the invention, a polynucleotide of the invention, or a cell population of the invention (e.g., to a cell-surface antigen).

In one aspect, the invention provides an antibody that selectively binds to a polypeptide marker described in Table 1, Table 2, Table 3, Table 4, Table 5 or Table 6, or that selectively binds to a molecule that comprises such a polypeptide marker.

In another embodiment, the invention provides an antibody that selectively binds to a polypeptide that is substantially homologous to a polypeptide marker described in Table 1, Table 2, Table 3, Table 4, Table 5 or Table 6, or that selectively binds to a molecule that comprises such a polypeptide.

In another embodiment, the invention provides an antibody that selectively binds to a polypeptide having an M+H value of about the value stated for a polypeptide marker described in Table 1, Table 2, Table 3, Table 4, Table 5 or Table 6, or that selectively binds to a molecule that comprises such a polypeptide.

In another embodiment, the invention provides an antibody that selectively binds to a polypeptide having an M+H value within 1% (more particularly within 0.5%, more particularly within 0.1%, more particularly, within 0.05%, more particularly within 0.01% of the M+H value stated for a polypeptide marker described in Table 1, Table 2, Table 3, Table 4, Table 5 or Table 6, or that selectively binds to a molecule that comprises such a polypeptide.

In another embodiment, the invention provides an antibody that selectively binds to a polypeptide that is a fragment, precursor, successor or modified version of a polypeptide marker described in Table 1, Table 2, Table 3, Table 4, Table 5 or Table 6, or that selectively binds to a molecule that comprises such a polypeptide.

In another embodiment, the invention provides an antibody that selectively binds to a polypeptide that is structurally different from a polypeptide marker described in Table 1, Table 2, Table 3, Table 4, Table 5 or Table 6 but is capable of performing the function of that polypeptide marker in a functional assay, or that selectively binds to a molecule that comprises such a polypeptide.

In another embodiment, the invention provides an antibody that selectively binds to a polynucleotide that encodes a polypeptide of the invention, or that selectively binds to a molecule that comprises such a polynucleotide.

In another embodiment, the invention provides an antibody that selectively binds to a polynucleotide that is a fragment or modified version or is substantially homologous to a polynucleotide that encodes a polypeptide of the invention, or that selectively binds to a molecule that comprises such a polynucleotide.

In another embodiment, the invention provides an antibody that selectively binds to a cell population of the invention. In a preferred embodiment, the antibody selectively binds to a molecule associated with a cell that is a member of a cell population of the invention; in another preferred embodiment, the cell-associated molecule is a surface antigen.

Certain antibodies that selectively bind polypeptides of the invention, polynucleotides of the invention, or cell populations and cell-associated molecules of the invention already may be known and/or available for purchase from commercial sources. Antibodies of the invention also may be prepared by any suitable means known in the art. For example, antibodies may be prepared by immunizing an animal host with a marker or an immunogenic fragment thereof (conjugated to a carrier, if necessary). Adjuvants, such as Freund's adjuvant optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker of the invention can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art. Examples of other methods used to identify antibodies include binding assays with random peptide libraries (e.g., phage display), systematic evolution of ligands by exponential enrichment (SELEX) and design methods based on an analysis of the structure of the targeted marker.

Antibodies that selectively bind markers of the invention can be used, for example, in methods to isolate or detect markers of the invention (e.g., a polypeptide described in Table 1, Table 2, Table 3, Table 4, Table 5 or Table 6, or a cell population described in Table 7 or Table 8) using methods and techniques well-known in the art. In some embodiments, for example, the antibodies are conjugated to a detection molecule or moiety (e.g., a dye, an enzyme) and can be used in ELISA or sandwich assays to detect markers of the invention.

In another embodiment, antibodies against a polypeptide of the invention, a polynucleotide of the invention, or a cell of a cell population of the invention can be used to assay a tissue sample for such marker. The antibodies can selectively bind any to marker present in the tissue sample sections and allow the localization of the marker in the tissue. Similarly, antibodies labeled with a radioisotope may be used for in vivo imaging or treatment applications. Techniques for conjugating antibodies to therapeutic or imaging agents are well known in the art.

VI. Methods of Diagnosing Rheumatoid Arthritis

The present invention includes all methods relying on correlations between the polypeptide markers, polynucleotide markers and cell population markers described herein and the presence of RA.

In one aspect, the invention provides methods for diagnosing RA in a subject. In one embodiment, the invention provides a method for determining whether a subject has RA. These methods comprise obtaining a biological sample from a subject suspected of having RA, or at risk for developing RA, detecting the level or activity of a marker of the invention in the sample, and comparing the result to the level or activity of the marker in a sample obtained from a non-RA subject, or to a standard level or reference range. Typically, the standard level or reference range is obtained by measuring the same marker or markers in a set of non-RA subjects. Measurement of the standard level or reference range need not be made contemporaneously; it may be a historical measurement. Preferably the non-RA subjects are matched to the subject with respect to some attribute(s) (e.g., age and/or sex). Depending upon the difference between the measured level and the standard level or reference range, the subject can be diagnosed as having RA or as not having RA.

In one embodiment, an increased level or activity of a marker of the invention in a sample obtained from a subject suspected of having RA, or at risk for developing RA, is indicative that the subject has or is at risk for developing RA. Markers appropriate for this embodiment include those that have been identified as increased in samples obtained from RA subjects compared with samples from non-RA subjects (e.g., the polypeptide markers described in Table 1, Table 3 or Table 5 or the cell population markers described in Table 7). Other appropriate markers for this embodiment will be apparent to one of skill in the art in light of the disclosure herein.

In another embodiment, a decreased level or activity of a marker of the invention in a sample obtained from a subject suspected of having RA, or at risk for developing RA, is indicative that the subject has or is at risk for developing RA. Markers appropriate for this embodiment include those that have been identified as decreased in samples obtained from RA subjects compared with samples from non-RA subjects (e.g., the polypeptide markers described in Table 2, Table 4 or Table 6 or the cell population markers described in Table 8). Other appropriate markers for this embodiment will be apparent to one of skill in the art in light of the disclosure herein.

As will be appreciated by one of skill in the art, the methods of the present invention may be used to evaluate fragments of a polypeptide marker listed in Table 1, Table 2, Table 3, Table 4, Table 5 or Table 6, as well as molecules that contain the entire polypeptide marker, or at least a significant portion thereof (e.g., measured unique epitope), and modifications of such markers. Accordingly, such fragments, larger molecules and modifications are included within the scope of the invention.

The methods of the invention may be used to make the diagnosis of RA, independent from other information such as the patient's symptoms, for example, as measured by the American College of Rheumatology (ACR) Criteria (Arnett et al., Arthritis Rheum. 31:315-324 (1988), or the results of other clinical or laboratory tests, such as X-rays of affected joints or previously known markers for RA reported in the literature (e.g., rheumatoid factor). However, the methods of the invention are preferably used in conjunction with such other data points. Similarly, more than one of the markers of the invention may be measured in combination. Measurement of the markers of the invention along with any other markers known in the art, including those not specifically listed herein, falls within the scope of the invention.

As will be apparent to those of ordinary skill in the art, the method described above is not limited to making an initial diagnosis of RA, but also is applicable to confirming a provisional diagnosis of RA or “ruling out” such a diagnosis.

What is presently referred to as RA may turn out to be a number of related but distinguishable conditions. For example, RA subjects can be divided into groups based on response to anti-TNF-α therapy. Additional classifications may be made, and these types may be further distinguished into subtypes. Any and all of the various forms of RA are intended to be within the scope of the invention. Indeed, by providing a method for subsetting patients based on marker measurement level, the compositions and methods of the invention may be used to reveal and define various forms of the disease.

Because a diagnosis is rarely based exclusively on the results of a single test, the methods of the invention may be used to determine whether a subject is more likely than not to have RA, or is more likely to have RA than to have another disease, based on the difference between the measured and standard level or reference range of the marker. Such ranges may be based on other factors such as age and gender. Thus, for example, a patient with a putative diagnosis of RA may be diagnosed as being “more likely” or “less likely” to have RA in light of the information provided by a method of the invention. If a plurality of markers are measured, at least one and up to all of the measured markers must differ, in the appropriate direction, for the subject to be diagnosed as having (or being more likely to have) RA.

Although markers of the invention were identified in serum and blood, any biological sample may be analyzed for the markers of the invention. Blood, including its constituents such as serum and plasma, and urine represent preferred biological samples for analysis because they are easy samples to obtain. Molecules present in serum are often also present in more easily obtainable fluids such as urine or sputum. Serum and urine also represent preferred biological samples as they are expected to be reflective of the systemic manifestations of the disease. In some embodiments, the level of a marker may be compared to the level of the same or another marker or some other constituent in a different tissue, fluid or biological compartment. Thus, a differential comparison may be made of a marker in synovial fluid and serum, for example. It is also within the scope of the invention to compare the level of a marker with the level of another marker or some other constituent within the same compartment. The marker may be detected in any biological sample obtained from the subject by any suitable method known in the art, see infra.

As stated above, some of the marker measurement values are higher in samples from RA patients, while others are lower. A significant difference in the appropriate direction in the measured value of one or more of the markers indicates that the patient has (or is more likely to have) RA. If only one marker is measured, then that value must increase or decrease to indicate RA. If more than one marker is measured, then a diagnosis of RA can be indicated by a change in only one marker, all markers, or any number in between. In some preferred embodiments, multiple markers are measured, and a diagnosis of RA is indicated by changes in multiple markers. Measurements can be of (i) a marker of the invention, (ii) a marker of the invention and another factor known to be associated with RA (e.g., joint tenderness); (iii) a plurality of markers comprising at least one marker of the invention and at least one previously known marker reported in the literature, or (iv) any combination of the foregoing. Furthermore, the amount of change in a marker level may be an indication of the relative likelihood of the presence of the disease.

The invention also provides methods for determining a subject's risk of developing RA. The method comprises obtaining a biological sample from a subject, detecting the level or activity of a marker of the invention in the sample, and comparing the result to the level or activity of the marker in a sample obtained from a non-RA subject, or to a standard level or reference range, wherein, an increase or decrease of the marker is correlated with the risk of developing RA.

The invention also provides methods for determining the stage or severity of RA. The method comprises obtaining a biological sample from a subject, detecting the level or activity of a marker in the sample, and comparing the result to the level or activity of the marker of the invention in a sample obtained from a non-RA subject, or to a standard level or reference range, wherein an increase or decrease of the activity or level of the marker is correlated with the age or severity of the disease.

In an alternative embodiment of the invention, a method is provided for monitoring an RA patient over time to determine whether the disease is progressing. The specific techniques used in implementing this embodiment are similar to those used in the embodiments described above. The method is performed by obtaining a biological sample, such as serum from the subject at a certain time (t₁); measuring the level of at least one of the markers of the invention in the biological sample; and comparing the measured level with the level measured with respect to a biological sample obtained from the subject at an earlier time (t₀). Depending upon the difference between the measured levels, it can be seen whether the marker level has increased, decreased, or remained constant over the interval (t₁−t₀). A further deviation of a marker in the direction indicating RA, or the measurement of additional increased or decreased RA markers, would suggest a progression of the disease during the interval. Subsequent sample acquisitions and measurements can be performed as many times as desired over a range of times t₂ to tₙ.

The ability to monitor a patient by making serial marker level determinations would represent a valuable clinical tool. Rather than the limited “snapshot” provided by a single evaluation, such monitoring would reveal trends in marker levels over time. In addition to indicating a progression of the disease, tracking the marker levels in a patient could be used to predict exacerbations or indicate the clinical course of the disease. For example, as will be apparent to one of skill in the art, the markers of the invention could be further investigated to distinguish between any or all of the known forms of RA (for example, responders and non-responders to anti-TNF-α therapy) or any later described types or subtypes of the disease. In addition, the sensitivity and specificity of the methods of the invention could be further investigated with respect to distinguishing RA from other autoimmune diseases, other diseases associated with arthritis or to predict relapse and remission.

Analogously, as described, infra, the markers of the invention can be used to assess the efficacy of a therapeutic intervention in a subject. The same approach described above would be used, except a suitable treatment would be started, or an ongoing treatment would be changed, before the second measurement (i.e., after t₀ and before t₁). The treatment can be any therapeutic intervention, such as drug administration, dietary restriction or surgery, and can follow any suitable schedule over any time period. The measurements before and after could then be compared to determine whether or not the treatment had an effect effective. As will be appreciated by one of skill in the art, the determination may be confounded by other superimposed processes (e.g., an exacerbation of the disease during the same period).

It is to be understood that any correlations between biological sample measurements of the markers of the invention and RA, as used for diagnosis of the disease or evaluating drug effect, are within the scope of the invention.

VII. Methods for Measuring

In the methods of the invention, levels and activity of polypeptides of the invention, polynucleotides of the invention, or cell populations of the invention are measured (or detected) using conventional techniques. The measurement may be quantitative or qualitative. The measurement may be absolute or relative. It should be noted that while one technique may be used to identify the marker, in practice, a different technique may be used to measure the level or activity of the marker. A wide variety of techniques are available, including without limitation mass spectrometry, chromatographic separations, 2-D gel separations, binding assays (e.g., immunoassays), hybridization assays, enzyme assays and competitive inhibition assays, immunofluorescence and cytometry. Any effective method in the art for measuring the level or activity of a polypeptide, polynucleotide or cell population marker of the invention is included in the invention. It is within the ability of one of ordinary skill in the art to determine which method would be most appropriate for measuring a specific marker. Thus, for example, a robust ELISA assay may be best suited for use in a physician's office while a measurement requiring more sophisticated instrumentation may be best suited for use in a clinical laboratory. Regardless of the method selected, it is important that the measurements be reproducible.

Mass spectrometry, which allows direct measurement of analytes with high sensitivity and reproducibility, advantageously can be used to measure polypeptide markers of the invention. A number of mass spectrometric methods are available and could be used to accomplish the measurement. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Moreover, mass spectrometers that allow time-of-flight (TOF) measurements have high accuracy and resolution and are able to measure low abundant species, even in complex matrices like serum or synovial fluid.

For polypeptide markers, quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another.

In addition, one- and two-dimensional gels have been used to separate polypeptides and quantify gel spots by silver staining, fluorescence or radioactive labeling. These differently stained spots have been detected using mass spectrometry, and identified by tandem mass spectrometry techniques.

In preferred embodiments, the polypeptide markers are measured using mass spectrometry in connection with a separation technology, such as liquid chromatography-mass spectrometry or gas chromatography-mass spectrometry. It is particularly preferable to couple reverse-phase liquid chromatography to high resolution, high mass accuracy ESI time-of-flight (TOF) mass spectroscopy. This allows spectral intensity measurement of a large number of biomolecules from a relatively small amount of any complex biological material without sacrificing sensitivity or throughput. Analyzing a sample by this method allows the marker (characterized by, for example, the M+H value, or the retention time and mass-to-charge ratio within the given experimental platform) to be determined and quantified.

As will be appreciated by one of skill in the art, many other separation technologies may be used in connection with mass spectrometry. For example, a vast array of separation columns are commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each component having a characteristic mass-to-charge ratio (m/z) and retention time (R.T.) within the given experimental platform. Each polypeptide will have a characteristic M+H value. As one of skill in the art will recognize, there may not be a one-to-one correspondence between components (each with a characteristic m/z and R.T. within the given experimental platform) and the polypeptides having a characteristic M+H value (i.e., the former typically will outnumber the latter). The presence of a peak with the m/z and RT of a marker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. In addition, deconvoluting software is available to separate overlapping peaks. The retention time depends to some degree on the conditions employed in performing the liquid chromatography separation. The preferred conditions, and the conditions used to obtain the retention times that appear in the Tables, are set forth in Example 2. The various polypeptides of the invention have a characteristic M+H value.

The better the mass assignment, the more accurate is the detection and measurement of the marker level in the sample. Thus, the mass spectrometer selected for this purpose preferably provides high mass accuracy and high mass resolution. The mass accuracy of a well-calibrated Micromass TOF instrument, for example, is reported to be approximately 2 mDa, with resolution m/Δm exceeding 5000.

In other preferred embodiments, the level of the polypeptide markers may be determined using a standard immunoassay, such as a sandwich ELISA using matched antibody pairs and chemiluminescent detection. Commercially available or custom monoclonal or polyclonal antibodies are typically used. However, the assay can be adapted for use with other reagents that selectively bind to the marker. Standard protocols and data analysis are used to determine the marker concentrations from the assay data.

A number of the assays discussed above employ an antibody that selectively binds to the marker. An antibody may be identified and produced by any method accepted in the art, as discussed, supra.

The polypeptide markers of the invention also may be measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

The intracellular levels of polypeptide markers can also be measured. Typical methodologies include protein extraction from a cell or tissue sample, followed by hybridization of a labeled probe (e.g., an antibody) specific for the target protein to the protein sample, and detection of the probe. The label group can be a radioisotope, a fluorescent compound, an enzyme, or an enzyme co-factor. Detection of specific polypeptides may also be assessed by gel electrophoresis or column chromatography, among many other techniques well known to those skilled in the art.

Measurement of the level of a polynucleotide marker may be made by any method known in the art. See, e.g., Sambrook et al., supra; Ausubel et al. (eds.), Current Protocols in Molecular Biology, John Wiley & Sons (1992).

Typical methodologies for RNA detection include RNA extraction from a cell or tissue sample, followed by hybridization of a labeled probe (e.g., a complementary polynucleotide) specific for the target RNA to the extracted RNA, and detection of the probe (e.g., Northern blotting). Detection of specific polynucleotides may also be assessed by gel electrophoresis, column chromatography, direct sequencing, or quantitative PCR, among many other techniques well known to those skilled in the art.

Detection of the presence or number of copies of all or a part of a polypeptide marker gene or polynucleotide of the invention may be performed using any method known in the art. Typically, it is convenient to assess the presence and/or quantity of a DNA or cDNA by Southern analysis, in which total DNA from a cell or tissue sample is extracted, is hybridized with a labeled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art.

Cell populations of the invention may be measured and characterized by any method or technique accepted in the art. Flow cytometry, for example, is a widely used means for analyzing the physical and chemical properties of cell populations. Monoclonal antibodies against specific cell-surface or intracellular antigens, conjugated to fluorescent dyes, can be used as probes to detect expression of cellular antigens. After staining a sample with one or more fluorescent probes (either singly or in combination) the cells are conducted by the rapidly flowing stream, one at a time, though a focused laser beam. Information about the cell (e.g., its type, structure, size) can be determined from the fluorescent signal, and the manner in which the cell interacts with and scatters the light from the laser beam. The resulting data is typically compiled in a computer file for subsequent analysis. Flow cytometry also can be used to physically separate cells with particular characteristics (“cell sorting”).

Alternatively, cell populations of the invention may be analyzed using microvolume laser scanning cytometry (MLSC). In MLSC, as with flow cytometry, fluorophore-labeled antibodies specific for cell surface antigens are used to identify, characterize, and enumerate specific leukocyte populations. In a preferred embodiment, the SurroScan™ MLSC is used to classify and quantify cell populations. See Dietz et al., U.S. Pat. No. 6,603,537 (issued Aug. 5, 2003); Dietz et al., U.S. Pat. No. 6,687,395 (issued Feb. 3, 2004), Walton et al., supra. The staining reaction can be done with essentially any cell suspension, including whole blood, and assays can be executed in homogeneous mode. Typically, quantitative dilution of the blood-antibody mixture is usually sufficient sample preparation eliminating the need to wash away the reagent, significantly reducing the time needed for sample preparation.

After staining, the cell-antibody mixtures are loaded into optical-quality capillary arrays. The leukocytes of interest distribute throughout the capillary and, in whole blood assays, float to the top of the red cell hematocrit. In order to operate with whole blood, fluorophores that can be excited in the red region (>600 nm) of the spectrum with a HeNe laser, such as Cy5, Cy5.5 and Cy7-APC, are preferred. White blood cells isolated following ficoll or erythrocyte-lysis can also be routinely analyzed.

Each capillary in the array is analyzed with the laser-based fluorescence-imaging instrument. In contrast to flow cytometry, the laser scans over stationary cells rather than cells flowing past the laser. A small cylindrical laser spot is scanned across the capillary in one direction while the capillary is translated relative to the optical system in a second direction. Typically three antibody reagents, each with a different fluorescent tag and each detected in a different channel, are used per assay. The capillary is imaged and fluorescent events detected. This is in contrast to flow cytometry where light scatter rather than fluorescence is usually the trigger parameter.

Peaks corresponding to antibody-labeled cells are identified with image processing software that produces a list-mode data file with parameters for every detected cell event. Norton et al., supra. Unlabeled cells i.e., erythrocytes and leukocytes not expressing the target antibodies, are not identified. Intensity data is compensated for spectral overlap, so the resultant values are proportional to the amount of dye-antibody reagent on each cell. The volume of the scan is precisely defined enabling absolute cell counts (cells per μL of blood) to be determined.

Assay panels may be devised to identify and enumerate hundreds of different cell types and cell-associated molecules that are relevant to immune, inflammatory and metabolic processes. In a preferred embodiment, each reagent cocktail typically contains one or two antibodies to the major cell populations—neutrophils, eosinophils, monocytes T-cells, B-cells, NK-cells, and platelets—and one or two antibodies to subsetting antigens which may indicate the functional state, activation state or adhesion characteristics of the population.

VIII. Method of Treatment

This invention also provides method for treating RA, as well as other diseases or conditions, by providing a therapeutic agent to a subject that increases or decreases the level or activity of at least one polypeptide of the invention, polynucleotide of the invention, or cell population of the invention.

In one embodiment, the method comprises administering a therapeutic agent to a subject that increases the level or activity of at least one polypeptide of the invention, polynucleotide of the invention or cell population of the invention that is decreased in samples obtained from RA subjects compared to samples obtained from non-RA subjects or to a standard level or reference range.

In another embodiment, the method comprises administering a therapeutic agent to a subject that decreases the level of at least one polypeptide of the invention, polynucleotide of the invention or cell population of the invention that is increased in samples obtained from RA subjects compared to samples obtained from non-RA subjects or to a standard level or reference range.

In another embodiment, the method further comprises first obtaining a sample from an RA subject, determining the presence, level or activity of at least one marker of the invention in the sample compared to samples obtained from a non-RA subject or to a standard value or a reference range. If the marker is increased in the sample obtained from the RA subject, a therapeutic agent that decreases the level of the marker is administered to the patient. If the marker is decreased in the sample obtained from the RA subject, a therapeutic agent that increases the level of the marker is administered to the subject.

Therapeutic agents include but are not limited to polypeptide markers, polynucleotide markers, molecules comprising polypeptide markers or polynucleotide markers, antibodies specific for polypeptides of the invention, polynucleotides of the invention, or cell populations of the invention, modulators of the level or activity of a polypeptide of the invention, polynucleotide of the invention or cell population marker of the invention or compositions comprising one or more of the foregoing.

Generally, the therapeutic agents used in the invention are administered to the subject in an effective amount. An “effective amount” is typically the amount that is sufficient to obtain beneficial or desired clinical results. The effective amount is generally determined by a physician with respect to a specific patient and is within the skill of one in the art. Factors that may be taken into account in determining an effective amount include those relating to the condition being treated (e.g., type, stage, severity) as well as those relating to the subject (e.g., age, sex, weight).

The level or activity of a polypeptide marker may be increased or decreased by any suitable technique or method known in the art. The level of a polypeptide marker may be increased by providing the polypeptide marker to a subject. Alternatively, the level of a polypeptide marker may be increased by providing a polynucleotide that encodes the polypeptide marker (e.g., gene therapy). For those polypeptide markers with enzymatic activity, compounds or molecules known to increase that activity may be provided to the subject.

The level of a polypeptide marker may be decreased by providing antibodies specific for the polypeptide marker to the subject. Alternatively, the level of a polypeptide marker may be decreased by providing a polynucleotide that is “anti-sense” to the polynucleotide that encodes the polypeptide marker, or that encodes dysfunctional proteins. For those polypeptide markers with enzymatic activity, compounds or molecules known to decrease that activity (e.g., inhibitor or antagonist).

Polynucleotides of the invention may also be used to specifically suppress gene expression by methods such as RNA interference (RNAi), which may also include cosuppression and quelling. This and other techniques of gene suppression are well known in the art. A review of this technique is found in Marx, Science 288:1370-1372 (2000). Specifically, polynucleotides of the invention are useful for generating gene constructs for silencing specific genes. Polynucleotides of the invention may be used to generate genetic constructs that encode a single self-complementary RNA sequence specific for one or more genes of interest. Genetic constructs and/or gene-specific self-complementary RNA sequences may be delivered by any conventional method known in the art. Within genetic constructs, sense and antisense sequences flank an intron sequence arranged in proper splicing orientation making use of donor and acceptor splicing sites. Alternative methods may employ spacer sequences of various lengths rather than discrete intron sequences to create an operable and efficient construct. During post-transcriptional processing of the gene construct product, intron sequences are spliced-out, allowing sense and antisense sequences, as well as splice junction sequences, to bind forming double-stranded RNA. Select ribonucleases bind to and cleave the double-stranded RNA, thereby initiating the cascade of events leading to degradation of specific mRNA gene sequences, and silencing specific genes. Alternatively, rather than using a gene construct to express the self-complementary RNA sequences, the gene-specific double-stranded RNA segments are delivered to one or more targeted areas to be internalized into the cell cytoplasm to exert a gene silencing effect. Using this cellular pathway of gene suppression, gene function may be studied and high-throughput screening of sequences may be employed to discover sequences affecting gene expression.

The level of a cell population may be increased or decreased by any suitable technique or method known in the art. The level of a cell population may be increased in a sample, for example, by providing an appropriate chemoattractant. Chemokines, for example, have been shown to control the migratory behavior of several cell types, including lymphocytes. Conversely, the level of a cell population may be decreased by providing to the subject antibodies specific for the cell population.

The therapeutic agents described herein may be administered alone or in combination with another therapeutic compound, or other form of treatment. The compounds may be administered to the subjects in any suitable manner known in the art (e.g., orally, topically, subcutaneously, intradermally, intramuscularly, intravenously, intra-arterially, intrathecally). Therapeutic agents of the invention may be combined with an excipient and formulated as tablets or capsules for oral administration. Polypeptides may be formulated for parenteral administeration to avoid denaturation by stomach acids. For polynucleotides, vectors may be constructed for administration to the subject by a virus or other carrier. In a typical embodiment, cDNA is delivered to target cells (e.g., bone marrow cells) that are later reintroduced into the subject for expression of the encoded protein.

The therapeutic agents of the invention can be administered by any suitable means, including, for example, parenteral, intravenous, topical, oral or local administration, such as intradermally, by aerosol, or by injection. A therapeutic composition can be administered in a variety of unit dosage forms depending upon the method of administration. For example, unit dosage forms suitable for oral administration of subject include powder, tablets, pills and capsules. For particular modes of delivery, a therapeutic composition of the invention can be formulated in an excipient of the invention. A therapeutic reagent of the invention can be administered to any subject, including a human, a non-human mammal or other non-human animal.

As one of skill in the art will appreciate, the particular mode of administration will depend on the condition to be treated. It is contemplated that administration of the agents of the invention may be via any suitable method known in the art.

Antibodies targeting cell populations of the invention advantageously may be administered by intravenous, interperitoneal, or subcutaneous injection, including administration to veins or the lymphatic system, or directly into the joint space.

In a further embodiment, the therapeutic agents of the invention are useful for gene therapy or gene delivery. As used herein, the phrases “gene therapy” or “gene delivery” refer to the transfer of genetic material (e.g., DNA or RNA) of interest into a host to treat or prevent a genetic or acquired disease or condition. The genetic material of interest encodes a product (e.g., a protein polypeptide, peptide or functional RNA) whose production in vivo is desired. For example, the genetic material of interest can encode a hormone, receptor, enzyme or polypeptide of therapeutic value. In a specific embodiment, the subject invention utilizes a class of lipid compounds for use in non-viral gene therapy which can complex with nucleic acids as described in Hughes, et al., U.S. Pat. No. 6,169,078 (issued Jan. 2, 2001), incorporated by reference herein in its entirety. These therapeutic compounds effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

IX. Therapeutic Compositions

Another aspect of the invention provides compositions comprising a polypeptide of the invention, a polynucleotide of the invention, an antibody against a polypeptide of the invention, polynucleotide of the invention, or cell population of the invention, an inhibitor of a polypeptide of the invention, polynucleotide of the invention, or cell population of the invention, or other molecule that can increase or decrease the level or activity of a polypeptide of the invention, polynucleotide of the invention or cell population of the invention. Such compositions may be pharmaceutical compositions formulated for use as a therapeutic.

In one embodiment, the invention provides a composition that comprises a polypeptide of the invention, including without limitation a polypeptide marker described in Table 1, Table 2, Table 3, Table 4, Table 5 or Table 6 or any of the other polypeptide markers of the invention described herein.

In one embodiment, the invention provides a composition that comprises a polynucleotide of the invention of the invention, including without limitation a polynucleotide that encodes a polypeptide marker described in Table 1, Table 2, Table 3, Table 4, Table 5, or Table 6 or any of the other nucleotides of the invention described herein.

In another embodiment, the invention provides a composition that comprises an antibody that selectively binds to a polypeptide of the invention, a polynucleotide of the invention or a cell population of the invention, or a molecule that comprises such an antibody.

In another embodiment, the invention provides a composition that comprises a modulator of the level or activity of a polypeptide of the invention, a polynucleotide of the invention, or cell population of the invention, or a molecule that comprises such a modulator. In one embodiment, the modulator is an inhibitor of a polypeptide of the invention. In another embodiment, the modulator is an antisense polynucleotide that is complementary to a polynucleotide that encodes a polypeptide of the invention.

Such compositions may be pharmaceutical compositions. Typically, a pharmaceutical composition comprises a therapeutically effective amount of an active agent and is formulated with a suitable excipient or carrier.

Generally, the therapeutic agents used in the invention are administered to the subject in an effective amount. Generally, an effective amount is an amount effective to either (1) reduce the symptoms of the disease sought to be treated or (2) induce a pharmacological change relevant to treating the disease sought to be treated. For RA, an effective amount includes an amount effective to: improve the DAS28 score, improve the American College of Rheumatology (ACR) functional scores, decrease tender and swollen joint counts, decrease duration of morning stiffness, and reduce any other objective or subjective indicia of the disease. Therapeutically effective amounts of the therapeutic agents will depend, in part, on the condition, type and location of the disease, the size and condition of the patient, as well as other factors readily known to those skilled in the art. The dosages can be given as a single dose, or as several doses, for example, divided over the course of several weeks.

The pharmaceutical compositions of the invention can be prepared in any suitable manner known in the pharmaceutical art. The carrier or excipient may be a solid, semisolid, or liquid material that can serve as a vehicle or medium for the active ingredient. Suitable carriers or excipients are well known in the art and include, but are not limited to saline, buffered saline, dextrose, water, glycerol, ethanol, and combinations thereof. The pharmaceutical compositions may be adapted for oral, inhalation, parenteral, or topical use and may be administered to the patient in the form of tablets, capsules, aerosols, inhalants, suppositories, solutions, suspensions, powders, syrups, and the like. As used herein, the term “pharmaceutical carrier” may encompass one or more excipients. Suitable pharmaceutical carriers and formulation techniques are found in standard texts, such as Remington's Pharmaceutical Sciences, Mack Publishing Co., Easton, Pa.

One embodiment of the invention is a controlled release formulation that is capable of slowly releasing a composition of the invention into an animal. As used herein, a controlled release formulation comprises a composition of the invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the invention include liquids that, upon administration to an animal, form a solid or a gel in situ. Preferred controlled release formulations are biodegradable (i.e., bioerodible).

X. Methods for Screening Candidate Compounds

In another aspect, the invention provides methods for screening candidate compounds for use as therapeutic agents. In one embodiment, the method comprises screening candidate compounds for those that bind to a polypeptide of the invention, a polynucleotide of the invention, or a cell population of the invention. Candidate compounds that bind to markers can be identified using any suitable method or technique known in the art.

In one embodiment, a candidate compound or a control is contacted with a marker of the invention and the ability of the candidate compound to form stable complexes with the marker is determined (e.g., flow cytometry, immunoprecipitation). The candidate compound, the marker, or an antibody that selectively binds either may be labeled to facilitate detection. The candidate molecule or marker may be immobilized on a solid support (e.g., a bead).

In another embodiment, cells expressing a polypeptide marker are contacted with a candidate compound or a control and the ability of the candidate compound to form stable complexes with the cells is determined. The candidate compound or the marker may be labeled to facilitate detection.

In another embodiment, the method comprises screening candidate compounds for those that have a stimulatory or inhibitory effect on the activity of a marker of the invention comprising comparing the activity of the marker in the presence of the candidate molecule with the activity of the marker in the absence of the candidate molecule (e.g., in the presence of a control).

In another embodiment, the method comprises screening candidate drugs in a clinical trial to determine whether a candidate drug is effective in treating RA. At time t₀, a biological sample is obtained from each subject in population of subjects diagnosed with RA. Next, assays are performed on each subject's sample to measure levels of a marker. In some embodiments, only a single marker is monitored, while in other embodiments, a combination of markers, up to the total number of factors, is monitored. Next, a predetermined dose of a candidate drug is administered to a portion or sub-population of the same subject population. Drug administration can follow any suitable schedule over any time period. In some cases, varying doses are administered to different subjects within the sub-population, or the drug is administered by different routes. At time t₁, after drug administration, a biological sample is acquired from the sub-population and the same assays are performed on the biological samples as were previously performed to obtain measurement values. As before, subsequent sample acquisitions and measurements can be performed as many times as desired over a range of times t₂ to tₙ. In such a study, a different sub-population of the subject population serves as a control group, to which a placebo is administered. The same procedure is then followed for the control group: obtaining the biological sample, processing the sample, and measuring the markers to obtain a measurement chart.

Specific doses and delivery routes can also be examined. The method is performed by administering the candidate drug at specified dose or delivery routes to subjects with RA; obtaining biological samples, such as serum, from the subjects; measuring the level of at least one of the markers in each of the biological samples; and, comparing the measured level for each sample with other samples and/or a standard level or reference range. Typically, the standard level or reference range is obtained by measuring the same marker or markers in the subject before drug administration. Depending upon the difference between the measured and standard levels, the drug can be considered to have an effect on RA. If multiple markers are measured, at least one and up to all of the markers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating RA.

In a typical embodiment, a subject population having RA is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed.

In general, a number of statistical considerations must be made in designing the trial to ensure that statistically significant changes in marker measurements can be detected following drug administration. The amount of change in a marker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. At time t₀, a biological sample is obtained from each subject in the population. Preferably, the sample is blood, but other biological fluids may be used (e.g., urine). Next, an assay or variety of assays are performed on each subject's sample to measure levels of particular markers of the invention. The assays can use conventional methods and reagents, as described above. If the sample is blood, then the assays typically are performed on either serum or plasma. For other fluids, additional sample preparation steps are included as necessary before the assays are performed. The assays measure values of at least one of the markers of the invention. In some embodiments, only a single marker is monitored, while in other embodiments, a combination of factors, up to the total number of markers, is monitored. The markers may also be monitored in conjunction with other measurements and factors associated with RA (e.g., joint tenderness). The number of markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. At time t₁, after drug administration, another biological sample (the “t₁ sample”) is acquired from the sub-population. Typically, the sample is the same type of sample and processed in the same manner (for example, blood) as the sample acquired from the subject population before drug administration (the “t₀ sample”). The same assays are performed on the t₁ sample as on the to sample t₀ obtain measurement values. Subsequent sample acquisitions and measurements can be performed as many times as desired over a range of times t₂ to tₙ.

Typically, a different sub-population of the subject population is used as a control group, to which a placebo is administered. The same procedure is then followed for the control group: obtaining the biological sample, processing the sample, and measuring the markers to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired measurements of the various markers are thus determined for each subject. The different measurement values are compared and analyzed to determine whether the markers changed in the expected direction for the drug group but not for the placebo group, indicating that the candidate drug is effective in treating RA. In preferred embodiments, such change is statistically significant. The measurement values at time t₁ for the group that received the candidate drug are compared with standard measurement values, preferably the measured values before the drug was given to the group, i.e., at time t₀. Typically, the comparison takes the form of statistical analysis of the measured values of the entire population before and after administration of the drug or placebo. Any conventional statistical method can be used to determine whether the changes in marker values are statistically significant. For example, paired comparisons can be made for each marker using either a parametric paired t-test or a non-parametric sign or sign rank test, depending upon the distribution of the data.

In addition, tests should be performed to ensure that statistically significant changes found in the drug group are not also found in the placebo group. Without such tests, it cannot be determined whether the observed changes occur in all patients and are therefore not a result of candidate drug administration.

As discussed, supra, some of the marker measurement values are higher in samples from RA patients, while others are lower. The nonadjusted p-values shown were obtained by univariate analysis. A significant change in the appropriate direction in the measured value of one or more of the markers indicates that the drug is effective. If only one marker is measured, then that value must increase or decrease to indicate drug efficacy. If more than one marker is measured, then drug efficacy can be indicated by change in only one marker, all markers, or any number in between. In some embodiments, multiple markers are measured, and drug efficacy is indicated by changes in multiple markers. Measurements can be of both markers of the invention and other measurements and factors associated with RA (e.g., measurement of previously known markers reported in the literature). Furthermore, the amount of change in a marker level may be an indication of the relatively efficacy of the drug.

In addition to determining whether a particular drug is effective in treating RA, markers of the invention can also be used to examine dose effects of a candidate drug. There are a number of different ways that varying doses can be examined. For example, different doses of a drug can be administered to different subject populations, and measurements corresponding to each dose analyzed to determine if the differences in the markers before and after drug administration are significant. In this way, a minimal dose required to effect a change can be estimated. In addition, results from different doses can be compared with each other to determine how each marker behaves as a function of dose.

Analogously, administration routes of a particular drug can be examined. The drug can be administered differently to different subject populations, and measurements corresponding to each administration route analyzed to determined if the differences in the markers before and after drug administration are significant. Results from the different routes can also be compared with each other directly.

XI. Kits

In another aspect, the invention provides a kit for detecting a polypeptide of the invention, a polynucleotide of the invention or a cell population of the invention.

In another aspect, the invention provides a kit for diagnosing RA in a patient by detecting at least one polypeptide of the invention, polynucleotide of the invention or cell population of the invention in a biological sample from the subject. In one embodiment, the kit is for monitoring progression of the disease. In another embodiment, the kit is for assessing response to therapy.

In another aspect, the invention provides a kit for screening candidate compounds by detecting stable complexes between the candidate compound and a polynucleotide of the invention, polynucleotide of the invention or cell population of the invention.

The kits of the invention may comprise one or more of the following: an antibody, wherein the antibody selectively binds to a polypeptide of the invention, polynucleotide of the invention or cell population of the invention, a labeled binding partner to the antibody (e.g., a “secondary antibody”), a solid phase upon which is immobilized the antibody or its binding partner, a polynucleotide probe that can hybridize to a polynucleotide marker, pairs of primers that under appropriate reaction conditions can prime amplification of at least a portion of a polynucleotide marker or a polynucleotide encoding a polypeptide marker (e.g., by PCR), instructions on how to use the kit, a container for a collected sample, or a label or insert indicating regulatory approval for diagnostic or therapeutic use.

In developing such kits, it is within the competence of one of ordinary skill in the art to perform validation studies that would use an optimal analytical platform for each marker. For a given marker, this may be an immunoassay, flow cytometer assay or mass spectrometry assay. Kit development may require specific antibody development, evaluation of the influence (if any) of matrix constituent (“matrix effects”), and assay performance specifications.

### EXAMPLES

### Example 1

### Clinical Study

The Institutional Review Board (IRB) approved protocol includes collection of samples from subjects with established RA (RA subjects) and non-RA subjects, matched for age gender and co-morbidities.

For the cell population analysis, RA subjects included individuals with a range of disease activity from remission to severe based on Disease Activity Scores. Specifically, the DAS28, a composite index of swollen and tender joints, erythrocyte sedimentation rate and general health, was used. van der Heijde et al., Ann. Rheum. Dis. 49:919-20 (1990); Prevoo et al., Arthritis Rheum. 38:44-8 (1995). Subject scores ranged from <2 to 7.7 (median 2.9) and ACR functional scores ranged from 1 to 4. Two cross sectional studies, with different panels of cellular assays compared 95 RA subjects and 30 non-RA subjects and 77 RA subjects and 48 non-RA subjects, respectivley.

For the mass spectrometry analysis, RA subjects included individuals with moderate to severe disease activity, with DAS28 scores ranging from 3.3 to 7.7 (median 5.2) and ACR functional scores of 3 or 4. The cross sectional study compared 20 RA subjects and 20 healthy subjects.

In both cases, serum samples were collected from RA and non-RA subjects in accordance with a clinical protocol and informed consent that were approved by an institutional review board (IRB) and with procedures that adhere to Good Clinical Practice.

### Example 2

### Mass Spectrometry Analysis

A high molecular weight fraction (“serum proteome”) was separated from the serum samples using a 5-kDa molecular weight cut-off spin filter (Millipore Corp., Bedford, Mass.). The serum proteome was diluted with PBS buffer (pH 6.0). To increase the effective dynamic range of the measurements, the two most abundant proteins (human serum albumin and IgG) were substantially depleted by an affinity resin (ProMetic Biosciences, Cambridge, UK). The remaining proteins were denatured using guanidine hydrochloride, disulfide bonds were reduced using dithioreitol, and sulfhydryl groups were carboxymethylated using iodoacetic acid/NaOH. The denaturant and reduction-alkylation reagents were removed by buffer exchange. After digestion of the proteins using modified Trypsin (Promega Corp., Madison, Wis.), the mixture was lyophilized to a powder, dissolved in formic acid, desalted, dried again, and redissolved in 0.1% formic acid for injection onto the liquid chromatography-mass spectrometer.

The tryptic peptides were profiled by liquid chromatography-electrospray ionization-mass spectrometry (LC-ESI-MS) on a high-resolution time-of-flight (TOF) instrument. For LC separation, an online column (PicoTip, New Objective) was packed with C18 reverse-phase (RP) material. Peptides retained on the RP column were eluted with increasing concentration of acetonitrile (ACN). A 100 minute gradient of H₂O/AcN was the basis of elution, going to 40% acetonitrile. The eluate from the column flowed into the ESI-TOF MS (Micromass LCT™, Waters Corp., Milford, Mass.). Individual molecules were tracked across samples and their differential expression determined.

A binary HP 1100 series HPLC was directly coupled to a MicroMass (Manchester, UK) LCT™ EST-TOF mass spectrometer equipped with a nanospray source (New Objective, Woburn, Mass.) for serum profiling or a ThermoFinnigan (San Jose, Calif.) LCQ DECA™ ESI ion-trap mass spectrometer for peptide identification. Details of the system set-up are described elsewhere. Wang et al., supra. Mass peaks were analyzed with MassView™ software (SurroMed, Inc., Menlo Park, Calif.), which tracks peaks and performs normalization to enable quantitative comparisons across multiple samples. Wang et al., supra; Hastings et al. Rapid Commun. Mass Spectrom., 16:462-7 (2002).

### Example 3

### Cell Population Analysis

Cellular assays were conducted on the SurroScan™ microvolume laser scanning cytometer (MLSC) using Flex32™ capillary arrays (SurroMed Inc., Menlo Park, Calif.). Walton et al., supra. The SurroScan system is based in part on the Imagn2000™ MLSC (Becton Dickinson, San Jose, Calif.). Dietz et al., Cytometry, 23177-86 (1996). However, in the SurroScan system (i) four colors can be analyzed instead of two, (ii) capillary arrays are used to enable many more assays and (iii) software enables streamlined data processing and connection to the database.

Monoclonal antibodies and fluorescent tags were obtained from commercial vendors (BD Biosciences, San Jose, Calif., including BD PharMingen, San Jose, Calif.; Beckman Coulter, Miami, Fla.; Serrotec, Raleigh, N.C.; and eBiosciences, San Diego, Calif.). Three different fluorophores were used as direct conjugates to the antibodies: Cy5, Cy5.5, and Cy7-APC. Mujumdar et al. Bioconjug. Chem. 4:105-11 (1993); Beavis & Pennline, Cytometry 24:390-394 (1996); Roederer et al., Cytometry, 24:191-7 (1996). Antibody-dye reagents were titrated to determine the appropriate concentration and combined into pre-made cocktails.

Images were converted to flow cytometry standard format with in-house software and analyzed with FlowJo™ cytometry analysis software customized for SurroMed (Tree Star, Inc., San Carlos, Calif.). Norton et al., supra. Fluorescence intensities were compensated for spectral overlap of the dyes so values would be proportional to antigen density. For the clinical study, list mode data is uploaded into an Oracle database and analyzed with in-house software using standard gates developed with the FlowJo and uploaded into the database.

About 800 cellular variables were analyzed, including cell counts, cell ratios and intensities. Some of these unique combinations were not independent and may represent the same or overlapping biological cell populations. For the major cell populations (neutrophils, eosinophils, monocytes, total T-cells, CD4 T-cells, CD8 T-cells, B-cells and NK cells) that were measured by an identical two-antigen combination (each with a different third antigen) in multiple assays, appropriate averages were calculated and used as a single variable for comparative statistics. Many of the cell populations in Table 7 and Table 8 are designated by the antigens used to define them where p=positive, n=negative, pn=dull, t=total in the assay. Thus, “CD3p” indicates a CD3 positive cell).

Whole blood assays results for T cell subsets; cell events can be displayed in histograms or dot plots based on the level of antigen expression. CD4 and CD8 T cells can be divided into naïve and memory T cell subsets. Four subsets can be identified and related to specific functional states: naïve (CD45RA⁺, CD62L⁺), central memory (CD45RA⁻, CD62L⁺), effector memory cells (CD45RA⁻, CD62L⁻) and terminal effector memory (CD45RA⁺, CD62L⁻) according to one scheme for CD8 T cells. Hamann et al., Intl. Immunol., 11:1027-1033 (1999); Sallusto et al., Nature, 401:708-712 (1999).

### Example 4

### Statistical Methods

Samples from RA subjects and healthy subject were analyzed with the cell population and mass spectrometry platforms to look for significant differences between the two groups. Variables were compared with either an un-paired t-test or non-parametric test, as appropriate for each variable, using SAS™ software. The study includes multiple comparisons and caution is needed to consider potential false positive conclusions. The step-down Bonferroni p-value adjustment method of Holm was used maintain a study-wide p-value <0.05. Results are considered at both the adjusted and multiple-univariate statistical levels. Holm, S., A simple sequentially rejective multiple test procedure, in Scand J Stat. 1979. p. 65-70; Blair, et al., Control of familywise errors in multiple endpoint assessments via stepwise permutation tests. 1996. 15(11): p. 1107-1121.

TABLE 1
Identified Full Length Proteins Increased in Subjects having RA

| RA | | | | | | | | | |
|---|---|---|---|---|---|---|---|---|---|
| Marker | Accession | | | # | # | <Exp | Fold | | |
| # | # | gi # | Protein Description | Components | Peptides | Ratio> | Change | <P> | <Score> |
| 109 | NP_056472.2 | 27881501 | ATP-binding cassette, | 1 | 1 | 1.04 | 1.04 | 3.55 × 10⁻² | 5154 |
| | | | sub-family A, mem- | | | | | | |
| | | | ber 12 isoform b; | | | | | | |
| | | | ATP-binding cassette A12 | | | | | | |
| | | | [Homo sapiens] | | | | | | |
| 20 | LPHUB | 71789 | apolipoprotein | 5 | 3 | 1.08 | 1.08 | 1.63 × 10⁻² | 6186 |
| | | | B-100 precursor - human | | | | | | |
| 13 | NP_000055.1 | 4557385 | complement component 3 | 7 | 6 | 1.11 | 1.11 | 2.28 × 10⁻² | 7592 |
| | | | precursor [Homo | | | | | | |
| | | | sapiens] | | | | | | |
| 30 | NP_001176.1 | 4502337 | alpha-2-glycoprotein 1, | 4 | 2 | 1.12 | 1.12 | 2.21 × 10⁻² | 5327 |
| | | | zinc; Alpha-2- | | | | | | |
| | | | glycoprotein, zinc | | | | | | |
| | | | [Homo sapiens] | | | | | | |
| 32 | NP_006211.1 | 5453896 | serine (or cysteine) | 3 | 2 | 1.13 | 1.13 | 9.91 × 10⁻⁴ | 4696 |
| | | | proteinase inhibitor, | | | | | | |
| | | | clade A (alpha-1 | | | | | | |
| | | | antiproteinase, antitrypsin), | | | | | | |
| | | | member 2; Protease | | | | | | |
| | | | inhibitor 1-like; protease | | | | | | |
| | | | inhibitor 1 (alpha-1- | | | | | | |
| | | | antitrypsin)-like [Homo | | | | | | |
| | | | sapiens] | | | | | | |
| 80 | NP_000574.1 | 9845255 | group-specific component | 1 | 1 | 1.16 | 1.16 | 1.12 × 10⁻² | 11431 |
| | | | (vitamin D bind- | | | | | | |
| | | | ing protein); hDBP | | | | | | |
| | | | [Homo sapiens] | | | | | | |
| 95 | NP_000286.2 | 21361198 | serine (or cysteine) | 2 | 1 | 1.17 | 1.17 | 2.99 × 10⁻² | 4055 |
| | | | proteinase inhibitor, | | | | | | |
| | | | clade A (alpha-1 | | | | | | |
| | | | antiproteinase, antitrypsin), | | | | | | |
| | | | member 1; Protease inhibitor | | | | | | |
| | | | (alpha-1-antitrypsin); protease | | | | | | |
| | | | inhibitor 1 (anti-elastase), | | | | | | |
| | | | alpha-1-antitrypsin [Homo sapiens] | | | | | | |
| 46 | P04004 | 139653 | Vitronectin precursor | 1 | 1 | 1.20 | 1.20 | 3.54 × 10⁻² | 13378 |
| | | | (Serum spreading fac- | | | | | | |
| | | | tor) (S-protein) (V75) | | | | | | |
| | | | [Contains: Vitronectin | | | | | | |
| | | | V65 subunit | | | | | | |
| 35 | NP_005742.4 | 22538387 | A kinase anchor protein | 2 | 2 | 1.20 | 1.20 | 1.11 × 10⁻² | 3677 |
| | | | 9 isoform 2; yotiao; | | | | | | |
| | | | A-kinase anchoring | | | | | | |
| | | | protein 450; AKAP120- | | | | | | |
| | | | like protein [ | | | | | | |
| 87 | NP_056039.1 | 14150229 | retinoblastoma-associated | 1 | 1 | 1.20 | 1.20 | 4.07 × 10⁻³ | 5543 |
| | | | protein RAP140 | | | | | | |
| | | | [Homo sapiens] | | | | | | |
| 47 | S23650 | 2120082 | retrovirus-related | 1 | 1 | 1.24 | 1.24 | 4.82 × 10⁻² | 5372 |
| | | | hypothetical protein II - | | | | | | |
| | | | human retrotransposon LINE-1 | | | | | | |
| 77 | NP_054790.1 | 7661976 | nuclear receptor | 1 | 1 | 1.26 | 1.26 | 1.88 × 10⁻² | 4810 |
| | | | coactivator RAP250; perox- | | | | | | |
| | | | isome proliferator-act; | | | | | | |
| | | | nuclear receptor coac- | | | | | | |
| | | | tivator RAP2 | | | | | | |
| 33 | JE0242 | 7438711 | Ig kappa chain NIG26 | 3 | 2 | 1.27 | 1.27 | 1.25 × 10⁻² | 17201 |
| | | | precursor - human | | | | | | |
| 23 | P02774 | 139641 | Vitamin D-binding | 3 | 3 | 1.29 | 1.29 | 2.45 × 10⁻² | 9854 |
| | | | protein precursor (DBF) | | | | | | |
| | | | (Group-specific | | | | | | |
| | | | component) (GC-globulin) | | | | | | |
| | | | (VDB) | | | | | | |
| 49 | C4HU | 2144577 | complement C4A precursor | 1 | 1 | 1.29 | 1.29 | 3.62 × 10⁻² | 15496 |
| | | | [validated] - | | | | | | |
| | | | human | | | | | | |
| 84 | NP_068774.1 | 11386179 | guanine nucleotide binding | 1 | 1 | 1.29 | 1.29 | 3.02 × 10⁻² | 2686 |
| | | | protein (G pro- | | | | | | |
| | | | tein), gamma transducing | | | | | | |
| | | | activity polypeptide | | | | | | |
| | | | 1 [Homo sapiens] | | | | | | |
| 93 | NP_624358.1 | 21264371 | nucleoporin 98kD isoform | 2 | 1 | 1.29 | 1.29 | 2.73 × 10⁻³ | 5421 |
| | | | 4; nucleoporin | | | | | | |
| | | | 98kD; Nup98-Nup96 | | | | | | |
| | | | precursor; GLFG- | | | | | | |
| | | | repeat containing | | | | | | |
| | | | nucleoporin [Homo | | | | | | |
| | | | sapiens] | | | | | | |
| 19 | NP_000629.2 | 18201911 | vitronectin precursor; | 7 | 4 | 1.30 | 1.30 | 1.35 × 10⁻² | 8182 |
| | | | serum spreading fac- | | | | | | |
| | | | tor; somatomedin B; | | | | | | |
| | | | complement S-protein | | | | | | |
| | | | [Homo sapiens] | | | | | | |
| 2 | P01009 | 1703025 | Alpha-1-antitrypsin | 61 | 28 | 1.31 | 1.31 | 1.26 × 10⁻² | 10798 |
| | | | precursor (Alpha-1 pro- | | | | | | |
| | | | tease inhibitor) | | | | | | |
| | | | (Alpha-1-antiproteinase) | | | | | | |
| | | | (PRO0684/PRO220 | | | | | | |
| 89 | NP_064620.2 | 18378731 | HMG-BOX transcription | 2 | 1 | 1.32 | 1.32 | 1.64 × 10⁻² | 2886 |
| | | | factor BBX; x 001 | | | | | | |
| | | | protein [Homo sapiens] | | | | | | |
| 67 | NP_004658.1 | 4758520 | hect domain and RLD 2 | 1 | 1 | 1.33 | 1.33 | 1.42 × 10⁻² | 2964 |
| | | | [Homo sapiens] | | | | | | |
| 107 | NP_000710.3 | 27597080 | calcium channel, | 1 | 1 | 1.34 | 1.34 | 1.78 × 10⁻² | 5196 |
| | | | voltage-dependent, L type, | | | | | | |
| | | | alpha 1C subunit | | | | | | |
| | | | [Homo sapiens] | | | | | | |
| 28 | P08697 | 112907 | Alpha-2-antiplasmin | 4 | 2 | 1.34 | 1.34 | 7.75 × 10⁻³ | 7171 |
| | | | precursor (Alpha-2- | | | | | | |
| | | | plasmin inhibitor) | | | | | | |
| | | | (Alpha-2-PI) (Alpha-2-AP) | | | | | | |
| 50 | Q99743 | 3914169 | Neuronal PAS domain | 1 | 1 | 1.34 | 1.34 | 2.28 × 10⁻² | 2228 |
| | | | protein 2 (Neuronal | | | | | | |
| | | | PAS2) (Member of PAS | | | | | | |
| | | | protein 4) (MOP4) | | | | | | |
| 45 | P22932 | 133500 | Retinoic acid receptor | 1 | 1 | 1.35 | 1.35 | 3.35 × 10⁻² | 5714 |
| | | | gamma-2 (RAR-gamma-2) | | | | | | |
| 14 | OMHU1B | 69990 | alpha-1-B-glycoprotein - human | 7 | 5 | 1.36 | 1.36 | 1.55 × 10⁻² | 10348 |
| 29 | P05546 | 123055 | Heparin cofactor II | 2 | 2 | 1.36 | 1.36 | 3.19 × 10⁻² | 4519 |
| | | | precursor (HC-II) (Prote- | | | | | | |
| | | | ase inhibitor leuserpin 2) | | | | | | |
| | | | (HLS2) | | | | | | |
| 11 | P01857 | 121039 | Ig gamma-1 chain C region | 13 | 8 | 1.37 | 1.37 | 2.57 × 10⁻² | 11550 |
| 69 | NP_005521.1 | 5031777 | isocitrate dehydrogenase 3 | 1 | 1 | 1.37 | 1.37 | 1.53 × 10⁻³ | 3619 |
| | | | (NAD+) alpha | | | | | | |
| | | | precursor; H-IDH alpha; | | | | | | |
| | | | isocitric dehydro- | | | | | | |
| | | | genase; isocitrate | | | | | | |
| | | | dehydrogenase [NAD] sub- | | | | | | |
| | | | unit alpha, mitochondrial; | | | | | | |
| | | | NAD+-specific | | | | | | |
| | | | ICDH; NAD(H)-specific | | | | | | |
| | | | isocitrate dehydro- | | | | | | |
| | | | genase alpha subunit | | | | | | |
| | | | precursor; isocitrate | | | | | | |
| | | | dehydrogenase (NAD+) | | | | | | |
| | | | alpha chain precur- | | | | | | |
| | | | sor [Homo sapiens] | | | | | | |
| 4 | KUHU | 1070458 | ferroxidase (EC 1.16.3.1) | 33 | 22 | 1.38 | 1.38 | 1.30 × 10⁻² | 8872 |
| | | | precursor [vali- | | | | | | |
| | | | dated] - human | | | | | | |
| 106 | XP_058831.3 | 27500444 | similar to zona | 1 | 1 | 1.38 | 1.38 | 2.09 × 10⁻² | 2952 |
| | | | pellucida binding protein | | | | | | |
| | | | [Homo sapiens] | | | | | | |
| 85 | NP_061819.2 | 12056473 | N-acetylneuraminic acid | 1 | 1 | 1.38 | 1.38 | 3.33 × 10⁻³ | 2985 |
| | | | phosphate synthase; | | | | | | |
| | | | sialic acid synthase; | | | | | | |
| | | | sialic acid phosphate | | | | | | |
| | | | synthase [Homo sapiens] | | | | | | |
| 73 | NP_009049.1 | 6005922 | triple functional domain | 1 | 1 | 1.39 | 1.39 | 3.76 × 10⁻³ | 3615 |
| | | | (PTPRF interacting) | | | | | | |
| | | | [Homo sapiens] | | | | | | |
| 76 | NP_055433.1 | 7657009 | deleted in bladder | 1 | 1 | 1.40 | 1.40 | 1.04 × 10⁻² | 2680 |
| | | | cancer chromosome re- | | | | | | |
| | | | gion candidate 1 [Homo | | | | | | |
| | | | sapiens] | | | | | | |
| 18 | NP_000087.1 | 4557485 | ceruloplasmin (ferroxidase); | 4 | 4 | 1.40 | 1.40 | 2.41 × 10⁻² | 8960 |
| | | | Ceruloplasmin | | | | | | |
| | | | [Homo sapiens] | | | | | | |
| 96 | NP_037533.2 | 21361440 | RAB3A interacting protein | 1 | 1 | 1.41 | 1.41 | 1.70 × 10⁻³ | 2501 |
| | | | (rabin3)-like 1 | | | | | | |
| | | | [Homo sapiens] | | | | | | |
| 97 | NP_055874.1 | 22035665 | talin 2 [Homo sapiens] | 2 | 1 | 1.41 | 1.41 | 1.32 × 10⁻² | 3952 |
| 37 | XP_209546.1 | 27481320 | similar to Ceruloplasmin | 3 | 2 | 1.42 | 1.42 | 8.20 × 10⁻³ | 7687 |
| | | | precursor (Ferroxi- | | | | | | |
| | | | dase) [Homo sapiens] | | | | | | |
| 10 | NP_000598.1 | 9257232 | orosomucoid 1 precursor; | 20 | 10 | 1.43 | 1.43 | 8.37 × 10⁻³ | 9788 |
| | | | Orosomucoid-1 | | | | | | |
| | | | (alpha-1-acid glycoprotein-1); | | | | | | |
| | | | alpha-1-acid | | | | | | |
| | | | glycoprotein 1 [ | | | | | | |
| 27 | S05270 | 87890 | Ig lambda chain | 2 | 2 | 1.44 | 1.44 | 2.17 × 10⁻² | 4533 |
| | | | precursor - human | | | | | | |
| 90 | NP_004886.2 | 19923284 | cold autoinflammatory | 2 | 1 | 1.44 | 1.44 | 3.41 × 10⁻³ | 4848 |
| | | | syndrome 1; chromo- | | | | | | |
| | | | some 1 open reading | | | | | | |
| | | | frame 7; angio- | | | | | | |
| | | | tensin/vasopressin recept | | | | | | |
| 104 | XP_044347.3 | 27499033 | similar to KIAA0913 | 1 | 1 | 1.44 | 1.44 | 1.05 × 10⁻² | 3764 |
| | | | protein [Homo | | | | | | |
| | | | sapiens] | | | | | | |
| 65 | NP_000326.1 | 4506809 | sodium channel, | 1 | 1 | 1.45 | 1.45 | 2.65 × 10⁻² | 2741 |
| | | | voltage-gated, type V, | | | | | | |
| | | | alpha polypeptide | | | | | | |
| | | | [Homo sapiens] | | | | | | |
| 78 | NP_060549.1 | 8922392 | hypothetical protein | 1 | 1 | 1.45 | 1.45 | 4.68 × 10⁻² | 3053 |
| | | | FLJ10379 [Homo | | | | | | |
| | | | sapiens] | | | | | | |
| 31 | NP_000599.1 | 4505529 | orosomucoid 2; | 4 | 2 | 1.46 | 1.46 | 2.14 × 10⁻² | 13328 |
| | | | alpha-1-acid glycoprotein, | | | | | | |
| | | | type 2 [Homo sapiens] | | | | | | |
| 21 | P01876 | 113584 | Ig alpha-1 chain C region | 4 | 3 | 1.46 | 1.46 | 2.62 × 10⁻² | 15257 |
| 55 | NP_001747.1 | 4502595 | corticosteroid binding | 1 | 1 | 1.47 | 1.47 | 5.70 × 10⁻³ | 10918 |
| | | | globulin precursor; | | | | | | |
| | | | corticosteroid binding | | | | | | |
| | | | globulin; alpha-1 anti- | | | | | | |
| | | | proteinase, antitrypsin | | | | | | |
| | | | [Homo sapiens] | | | | | | |
| 44 | P18136 | 125819 | KV3M_HUMAN IG KAPPA | 1 | 1 | 1.47 | 1.47 | 2.26 × 10⁻² | 3923 |
| | | | CHAIN V-III | | | | | | |
| | | | REGION HIC PRECURSOR | | | | | | |
| 22 | P01871 | 127514 | MUC_HUMAN Ig mu chain | 3 | 3 | 1.50 | 1.50 | 2.68 × 10⁻² | 11044 |
| | | | C region | | | | | | |
| 102 | XP_208769.1 | 27482513 | similar to Ig gamma-2 | 1 | 1 | 1.51 | 1.51 | 4.40 × 10⁻² | 6233 |
| | | | chain C region [Homo | | | | | | |
| | | | sapiens] | | | | | | |
| 51 | NP_001076.1 | 4501843 | alpha-1-antichymotrypsin, | 2 | 1 | 1.53 | 1.53 | 2.09 × 10⁻² | 12502 |
| | | | precursor; alpha- | | | | | | |
| | | | 1-antichymotrypsin; | | | | | | |
| | | | antichymotrypsin | | | | | | |
| | | | [Homo sapiens] | | | | | | |
| 68 | NP_005112.1 | 4827044 | thyroid hormone | 1 | 1 | 1.55 | 1.55 | 7.07 × 10⁻⁴ | 2376 |
| | | | receptor-associated protein, | | | | | | |
| | | | 240 kDa subunit | | | | | | |
| | | | [Homo sapiens] | | | | | | |
| 38 | S15590 | 106378 | Ig heavy chain - human | 1 | 1 | 1.55 | 1.55 | 2.58 × 10⁻² | 12558 |
| 7 | P01011 | 112874 | Alpha-1-antichymotrypsin | 17 | 11 | 1.57 | 1.57 | 7.69 × 10⁻³ | 9697 |
| | | | precursor (ACT) | | | | | | |
| 98 | XP_173158.1 | 22052041 | hypothetical | 1 | 1 | 1.57 | 1.57 | 2.59 × 10⁻² | 2573 |
| | | | protein XP_173158 [Homo | | | | | | |
| | | | sapiens] | | | | | | |
| 94 | NP_112494.2 | 21314742 | hypothetical protein | 1 | 1 | 1.60 | 1.60 | 4.65 × 10⁻³ | 2618 |
| | | | DKFZp434G2226 | | | | | | |
| | | | [Homo sapiens] | | | | | | |
| 3 | NP_005134.1 | 4826762 | haptoglobin [Homo sapiens] | 57 | 28 | 1.60 | 1.60 | 1.42 × 10⁻² | 10657 |
| 43 | P05155 | 124096 | Plasma protease C1 | 1 | 1 | 1.61 | 1.61 | 3.69 × 10⁻³ | 7962 |
| | | | inhibitor precursor (C1 | | | | | | |
| | | | Inh) (C1Inh) | | | | | | |
| 42 | P00737 | 123507 | Haptoglobin-1 precursor | 2 | 1 | 1.61 | 1.61 | 2.10 × 10⁻² | 24108 |
| 9 | NP_443204.1 | 16418467 | leucine-rich | 14 | 11 | 1.62 | 1.62 | 1.21 × 10⁻² | 9467 |
| | | | alpha-2-glycoprotein [Homo | | | | | | |
| | | | sapiens] | | | | | | |
| 64 | NP_000532.1 | 4506781 | S-arrestin; S-antigen | 1 | 1 | 1.66 | 1.66 | 1.48 × 10⁻² | 3677 |
| | | | [Homo sapiens] | | | | | | |
| 63 | NP_000895.1 | 4505417 | NAD(P)H dehydrogenase, | 1 | 1 | 1.70 | 1.70 | 1.41 × 10⁻² | 3103 |
| | | | quinone 2; NAD(P)H | | | | | | |
| | | | menadione oxidoreductase-1, di- | | | | | | |
| | | | oxin-inducible-2; | | | | | | |
| | | | NAD(P)H menadione oxi- | | | | | | |
| | | | doreductase 2, | | | | | | |
| | | | dioxin-inducible [Homo | | | | | | |
| | | | sapiens] | | | | | | |
| 48 | ANHU | 2144576 | angiotensin precursor | 1 | 1 | 1.70 | 1.70 | 7.03 × 10⁻³ | 2128 |
| | | | [validated] - human | | | | | | |
| 92 | XP_057927.2 | 20535708 | similar to KIAA1902 | 1 | 1 | 1.75 | 1.75 | 4.58 × 10⁻³ | 5802 |
| | | | protein [Homo | | | | | | |
| | | | sapiens] | | | | | | |
| 101 | XP_043492.2 | 27477685 | similar to KIAA1728 | 2 | 1 | 1.75 | 1.75 | 1.22 × 10⁻² | 2702 |
| | | | protein [Homo | | | | | | |
| | | | sapiens] | | | | | | |
| 108 | NP_775111.1 | 27765076 | calpain 3 isoform d; | 1 | 1 | 1.77 | 1.77 | 1.74 × 10⁻² | 3103 |
| | | | calpain, large polypep- | | | | | | |
| | | | tide L3; calpain p94, | | | | | | |
| | | | large [catalytic] subunit; | | | | | | |
| | | | muscle-specific | | | | | | |
| | | | calcium-activated neutral | | | | | | |
| | | | protease 3 large | | | | | | |
| | | | subunit [Homo sapiens] | | | | | | |
| 100 | NP_060606.2 | 24211029 | asp (abnormal spindle)- | 1 | 1 | 1.77 | 1.77 | 2.85 × 10⁻³ | 2838 |
| | | | like, microcephaly | | | | | | |
| | | | associated [Homo sapiens] | | | | | | |
| 36 | NP_066275.2 | 23821019 | haptoglobin-related | 5 | 2 | 1.83 | 1.83 | 1.43 × 10⁻² | 13046 |
| | | | protein; Haptoglobin- | | | | | | |
| | | | related locus [Homo sapiens] | | | | | | |
| 39 | P01877 | 113585 | Ig alpha-2 chain C region | 1 | 1 | 1.88 | 1.88 | 1.39 × 10⁻² | 10360 |
| 83 | T46372 | 11360168 | hypothetical protein | 3 | 1 | 2.07 | 2.07 | 1.00 × 10⁻² | 6240 |
| | | | DKFZp434P1818.1 - | | | | | | |
| | | | human (fragment) | | | | | | |
| 41 | P01860 | 121045 | GC3_HUMAN Ig gamma-3 | 1 | 1 | 2.34 | 2.34 | 6.35 × 10⁻⁴ | 3746 |
| | | | chain C region | | | | | | |
| | | | (Heavy chain disease | | | | | | |
| | | | protein) (HDC) | | | | | | |

TABLE 2
Identified Full Length Proteins Decreased in Subjects having RA

| RA | | | | | | | | | |
|---|---|---|---|---|---|---|---|---|---|
| Marker | Accession | | | # | # | <Exp | Fold | | |
| # | # | gi # | Protein Description | Components | Peptides | Ratio> | Change | <P> | <Score> |
| 16 | NP_000362.1 | 4507725 | transthyretin (prealbumin, | 8 | 5 | 0.95 | −1.05 | 1.65 × 10⁻² | 11714 |
| | | | amyloidosis type | | | | | | |
| | | | I); Transthyretin (prealbumin) | | | | | | |
| | | | [Homosapiens] | | | | | | |
| 24 | NP_000497.1 | 4503635 | coagulation factor II | 3 | 3 | 0.93 | −1.08 | 1.60 × 10⁻² | 10328 |
| | | | precursor; prothrombin | | | | | | |
| | | | [Homo sapiens] | | | | | | |
| 86 | NP_001398.1 | 13325066 | cadherin EGF LAG seven-pass | 1 | 1 | 0.85 | −1.17 | 4.67 × 10⁻² | 3976 |
| | | | G-type re- | | | | | | |
| | | | ceptor 3; EGF-like-domain, | | | | | | |
| | | | multiple 1; epi- | | | | | | |
| | | | dermal growth factor | | | | | | |
| 1 | NP_001054.1 | 4557871 | transferrin [Homo sapiens] | 73 | 47 | 0.85 | −1.18 | 1.63 × 10⁻² | 12997 |
| 99 | P57071 | 23503097 | PRDF_HUMAN PR-domain zinc | 1 | 1 | 0.84 | −1.19 | 1.73 × 10⁻² | 2646 |
| | | | finger pro- | | | | | | |
| | | | tein 15 (Zinc finger | | | | | | |
| | | | protein 298) | | | | | | |
| 59 | NP_002206.1 | 4504781 | inter-alpha (globulin) | 1 | 1 | 0.84 | −1.19 | 2.50 × 10⁻² | 5952 |
| | | | inhibitor, H1 polypep- | | | | | | |
| | | | tide [Homo sapiens] | | | | | | |
| 103 | XP_210868.1 | 27498981 | hypothetical protein | 1 | 1 | 0.84 | −1.20 | 2.03 × 10⁻² | 2548 |
| | | | XP_210868 [Homo | | | | | | |
| | | | sapiens] | | | | | | |
| 75 | T14760 | 7512615 | hypothetical protein | 1 | 1 | 0.83 | −1.20 | 2.71 × 10⁻² | 2794 |
| | | | DKFZp434I213.1 - hu- | | | | | | |
| | | | man (fragment) | | | | | | |
| 79 | NP_060835.1 | 8922950 | hypothetical protein | 2 | 1 | 0.83 | −1.20 | 2.17 × 10⁻² | 2758 |
| | | | FLJ11222 [Homo | | | | | | |
| | | | sapiens] | | | | | | |
| 34 | NP_009117.2 | 21735548 | centrosomal protein 2; | 3 | 2 | 0.83 | −1.20 | 2.65 × 10⁻² | 5099 |
| | | | centrosome associ- | | | | | | |
| | | | ated protein; centrosomal | | | | | | |
| | | | Nek2-associated | | | | | | |
| | | | protein 1 [Homo s | | | | | | |
| 60 | NP_000884.1 | 4504893 | kininogen [Homo sapiens] | 1 | 1 | 0.82 | −1.21 | 1.65 × 10⁻² | 3783 |
| 6 | NP_000468.1 | 4502027 | albumin precursor; PRO0883 | 29 | 20 | 0.82 | −1.22 | 1.67 × 10⁻² | 13267 |
| | | | protein [Homo | | | | | | |
| | | | sapiens] | | | | | | |
| 105 | XP_208509.1 | 27499046 | hypothetical protein | 1 | 1 | 0.82 | −1.22 | 3.36 × 10⁻² | 3549 |
| | | | XP_208509 [Homo | | | | | | |
| | | | sapiens] | | | | | | |
| 81 | NP_000604.1 | 11321561 | hemopexin [Homo sapiens] | 1 | 1 | 0.81 | −1.23 | 3.35 × 10⁻² | 13690 |
| 5 | NP_000005.1 | 4557225 | alpha 2 macroglobulin | 27 | 21 | 0.80 | −1.26 | 1.92 × 10⁻² | 9829 |
| | | | precursor [Homo | | | | | | |
| | | | sapiens] | | | | | | |
| 56 | NP_001422.1 | 4503579 | erythrocyte membrane | 1 | 1 | 0.79 | −1.26 | 1.93 × 10⁻² | 3089 |
| | | | protein band 4.1-like | | | | | | |
| | | | 2 [Homo sapiens] | | | | | | |
| 72 | NP_009185.1 | 6005836 | polynucleotide kinase | 2 | 1 | 0.79 | −1.26 | 2.72 × 10⁻² | 4218 |
| | | | 3′-phosphatase; | | | | | | |
| | | | polynucleotide kinase | | | | | | |
| | | | 3-prime-phosphatase | | | | | | |
| | | | [Homo sapiens] | | | | | | |
| 17 | NP_002207.1 | 4504783 | inter-alpha (globulin) | 4 | 4 | 0.79 | −1.26 | 3.22 × 10⁻² | 5550 |
| | | | inhibitor, H2 polypep- | | | | | | |
| | | | tide [Homo sapiens] | | | | | | |
| 74 | JE0243 | 7438712 | Ig kappa chain NIG93 | 1 | 1 | 0.79 | −1.26 | 3.05 × 10⁻² | 3177 |
| | | | precursor - human | | | | | | |
| 88 | NP_056986.2 | 15147337 | progestin induced | 1 | 1 | 0.79 | −1.27 | 1.82 × 10⁻² | 2705 |
| | | | protein; ubiquitin-protein | | | | | | |
| | | | ligase [Homo sapiens] | | | | | | |
| 61 | NP_002334.1 | 4505043 | lactotransferrin | 1 | 1 | 0.78 | −1.28 | 1.28 × 10⁻² | 3510 |
| | | | [Homo sapiens] | | | | | | |
| 52 | NP_001613.1 | 4502005 | alpha-2-HS-glycoprotein; | 1 | 1 | 0.78 | −1.28 | 3.60 × 10⁻³ | 10104 |
| | | | Alpha-2HS- | | | | | | |
| | | | glycoprotein [Homo sapiens] | | | | | | |
| 70 | NP_006613.1 | 5730055 | serum-inducible kinase | 1 | 1 | 0.76 | −1.31 | 1.32 × 10⁻³ | 3578 |
| | | | [Homo sapiens] | | | | | | |
| 15 | NP_000479.1 | 4502261 | serine (or cysteine) | 6 | 5 | 0.76 | −1.32 | 1.87 × 10⁻² | 9734 |
| | | | proteinase inhibitor, | | | | | | |
| | | | clade C (antithrombin), | | | | | | |
| | | | member 1; anti- | | | | | | |
| | | | thrombin III [Homo | | | | | | |
| 8 | P19823 | 125000 | Inter-alpha-trypsin | 13 | 11 | 0.76 | −1.32 | 1.43 × 10⁻² | 9269 |
| | | | inhibitor heavy chain H2 | | | | | | |
| | | | precursor (ITI heavy | | | | | | |
| | | | chain H2) (Inter-alpha- | | | | | | |
| | | | inhibitor 7 | | | | | | |
| 53 | NP_001624.1 | 4502067 | alpha-1-microglobulin/bikunin | 1 | 1 | 0.75 | −1.33 | 2.84 × 10⁻² | 2946 |
| | | | precursor;Alpha-1- | | | | | | |
| | | | microglobulin/bikunin precursor; | | | | | | |
| | | | inter-alpha-trypsin; Alpha-1- | | | | | | |
| | | | microglobulin/bikunin precursor | | | | | | |
| | | | (inter-alpha-trypsin inhibitor, | | | | | | |
| | | | light chain; protein | | | | | | |
| | | | HC) [Homo sapiens] | | | | | | |
| 26 | NP_000030.1 | 4557321 | apolipoprotein A-I | 4 | 3 | 0.75 | −1.33 | 3.59 × 10⁻² | 13128 |
| | | | precursor [Homo | | | | | | |
| | | | sapiens] | | | | | | |
| 71 | NP_006735.1 | 5803139 | retinol-binding protein 4, | 1 | 1 | 0.75 | −1.34 | 6.13 × 10⁻⁴ | 14481 |
| | | | plasma precursor; | | | | | | |
| | | | retinol-binding protein 4, | | | | | | |
| | | | plasma; retinol- | | | | | | |
| | | | binding protein 4, | | | | | | |
| | | | interstitial [Homo sapiens] | | | | | | |
| 82 | T46477 | 11360087 | hypothetical protein | 1 | 1 | 0.74 | −1.35 | 3.29 × 10⁻² | 2830 |
| | | | DKFZp434K1831.1- | | | | | | |
| | | | human (fragment) | | | | | | |
| 54 | NP_001634.1 | 4502149 | apolipoprotein A-II | 1 | 1 | 0.70 | −1.44 | 2.86 × 10⁻² | 2228 |
| | | | precursor [Homo | | | | | | |
| | | | sapiens] | | | | | | |
| 91 | NP_064547.2 | 20336302 | DEAD/H (Asp-Glu-Ala-Asp/His) | 1 | 1 | 0.69 | −1.45 | 1.85 × 10⁻² | 2878 |
| | | | box poly-peptide 33 | | | | | | |
| | | | [Homo sapiens] | | | | | | |
| 40 | P01859 | 121043 | Ig gamma-2 chain C region | 2 | 1 | 0.67 | −1.50 | 3.35 × 10⁻² | 13050 |
| 66 | NP_000578.1 | 4557387 | complement component 7 | 1 | 1 | 0.65 | −1.54 | 1.78 × 10⁻³ | 6388 |
| | | | precursor [Homo | | | | | | |
| | | | sapiens] | | | | | | |
| 62 | NP_002336.1 | 4505047 | lumican [Homo sapiens] | 1 | 1 | 0.65 | −1.55 | 1.77 × 10⁻⁶ | 8166 |
| 25 | NP_000549.1 | 4504347 | alpha 1 globin [Homo sapiens] | 4 | 3 | 0.62 | −1.60 | 1.97 × 10⁻² | 8520 |
| 12 | NP_000509.1 | 4504349 | beta globin [Homo sapiens] | 10 | 7 | 0.62 | −1.61 | 1.54 × 10⁻² | 7791 |
| 58 | NP_000510.1 | 4504351 | delta globin [Homo sapiens] | 1 | 1 | 0.59 | −1.68 | 1.03 × 10⁻² | 19814 |
| 57 | NP_000168.1 | 4504165 | gelsolin (amyloidosis, | 1 | 1 | 0.14 | −7.09 | 8.16 × 10⁻³ | 4055 |
| | | | Finnish type); Gel- | | | | | | |
| | | | solin [Homo sapiens] | | | | | | |

TABLE 3
Identified Protein Fragments Increased in Subjects having RA (C* signifies
carboxymethylation of C residue; M# signifies oxidation of M residue)

| RA | | | | | | | | | SEQ |
|---|---|---|---|---|---|---|---|---|---|
| Marker | Fragment | | R.T. | | | | Fold | P | ID |
| # | # | m/z | (min.) | z | M + H | Peptide | Change | used | NO: |
| 1 | 266 | 355.66 | 13.26 | 2 | 710.31 | DSSLCK | 1.58 | P < 0.05 | 185 |
| 1 | 1821 | 570.60 | 53.00 | 3 | 1709.78 | LCMGSGLNLCEPNNK | 1.45 | P < 0.01 | 184 |
| 1 | 3552 | 633.27 | 85.62 | 4 | 2530.06 | SMGGKEDLIWELLNQAQEHFGK | 1.42 | P < 0.05 | 183 |
| 1 | 1349 | 500.72 | 39.92 | 2 | 1000.43 | YLGEEYVK | 1.39 | P < 0.001 | 182 |
| 1 | 4360 | 550.99 | 55.39 | 4 | 2200.94 | SDNC*EDTPEAGYFAVAVVKK | 1.36 | P < 0.05 | 181 |
| 1 | 1274 | 489.49 | 61.98 | 4 | 1954.94 | NLNEKDYELLCLDGTR | 1.33 | P < 0.05 | 23 |
| 1 | 2860 | 854.91 | 48.87 | 2 | 1708.81 | LCMGSGLNLCEPNNK | 1.32 | P < 0.05 | 180 |
| 1 | 1818 | 570.27 | 48.92 | 3 | 1708.79 | LCMGSGLNLCEPNNK | 1.23 | P < 0.05 | 180 |
| 1 | 1494 | 520.72 | 24.11 | 2 | 1040.43 | FSEGCAPGSK | 1.21 | P < 0.05 | 179 |
| 1 | 1816 | 570.25 | 55.69 | 3 | 1708.73 | LCMGSGLNLCEPNNK | 1.16 | P < 0.05 | 180 |
| 1 | 768 | 427.96 | 48.84 | 4 | 1708.82 | LCMGSGLNLCEPNNK | 1.16 | CountDiff | 180 |
| 1 | 4725 | 1000.42 | 39.92 | 1 | 1000.42 | YLGEEYVK | 1.04 | CountDiff | 182 |
| 1 | 3844 | 794.86 | 35.79 | 2 | 1588.71 | KPVEEYANCHLAR | 1.02 | CountDiff | 8 |
| 2 | 3120 | 1321.19 | 74.14 | 3 | 3961.55 | MFNIQHCKKLSSWVLLMKYLGNA- | 1.94 | P < 0.05 | 211 |
| | | | | | | TAIFFLPDEGK | | | |
| 2 | 4107 | 1093.51 | 50.31 | 2 | 2186.01 | LYHSEAFTVNFGDTEEAKK | 1.62 | P < 0.001 | 210 |
| 2 | 2571 | 729.34 | 50.37 | 3 | 2186.00 | LYHSEAFTVNFGDTEEAKK | 1.60 | P < 0.001 | 210 |
| 2 | 1516 | 523.26 | 98.21 | 4 | 2090.02 | ELDRDTVFALVNYIFFK | 1.59 | P < 0.05 | 209 |
| 2 | 2448 | 697.35 | 98.24 | 3 | 2090.03 | ELDRDTVFALVNYIFFK | 1.57 | P < 0.05 | 209 |
| 2 | 2042 | 605.31 | 25.22 | 1 | 605.31 | VPMMK | 1.53 | P < 0.001 | 208 |
| 2 | 4617 | 729.32 | 52.07 | 3 | 2185.94 | LYHSEAFTVNFGDTEEAKK | 1.51 | P < 0.05 | 210 |
| 2 | 936 | 445.25 | 44.56 | 3 | 1333.73 | LVDKFLEDVKK | 1.47 | P < 0.05 | 207 |
| 2 | 3115 | 1288.15 | 71.37 | 2 | 2575.29 | TLNQPDSQLQLTTGNGLFLSEGLK | 1.46 | P < 0.05 | 206 |
| 2 | 2032 | 602.84 | 59.18 | 2 | 1204.67 | KLSSWVLLMK | 1.45 | P < 0.001 | 205 |
| 2 | 297 | 360.50 | 28.99 | 3 | 1079.48 | FLENEDRR | 1.44 | P < 0.001 | 204 |
| 2 | 157 | 336.86 | 27.66 | 3 | 1008.56 | QINDYVEK | 1.43 | P < 0.005 | 203 |
| 2 | 2967 | 946.42 | 47.25 | 2 | 1891.83 | DTEEEDFHVDQVTTVK | 1.43 | P < 0.05 | 202 |
| 2 | 298 | 360.50 | 27.54 | 3 | 1079.48 | FLENEDRR | 1.41 | P < 0.001 | 204 |
| 2 | 3074 | 1130.05 | 80.25 | 2 | 2259.09 | GTEAAGAMFLEAIPMSIPPEVK | 1.40 | P < 0.05 | 201 |
| 2 | 1558 | 529.73 | 14.49 | 2 | 1058.45 | EDPQGDAAQK | 1.40 | P < 0.001 | 200 |
| 2 | 871 | 438.00 | 50.28 | 5 | 2185.97 | LYHSEAFTVNFGDTEEAKK | 1.40 | P < 0.01 | 210 |
| 2 | 1385 | 505.23 | 33.46 | 2 | 1009.45 | QINDYVEK | 1.39 | P < 0.001 | 203 |
| 2 | 3022 | 1008.49 | 31.34 | 1 | 1008.49 | QINDYVEK | 1.39 | P < 0.005 | 203 |
| 2 | 4045 | 992.45 | 40.70 | 1 | 992.45 | QINDYVEK | 1.36 | P < 0.01 | 203 |
| 2 | 2899 | 888.49 | 35.91 | 1 | 888.49 | AVLTIDEK | 1.36 | P < 0.005 | 199 |
| 2 | 2183 | 631.27 | 47.32 | 3 | 1891.79 | DTEEEDFHVDQVTTVK | 1.35 | P < 0.005 | 202 |
| 2 | 1658 | 545.77 | 40.78 | 2 | 1090.53 | WERPFEVK | 1.35 | P < 0.005 | 198 |
| 2 | 2856 | 852.48 | 31.84 | 1 | 852.48 | SASLHLPK | 1.34 | P < 0.005 | 197 |
| 2 | 765 | 426.74 | 31.81 | 2 | 852.47 | SASLHLPK | 1.33 | P < 0.001 | 196 |
| 2 | 2710 | 779.40 | 37.54 | 1 | 779.40 | SPLFMGK | 1.31 | P < 0.005 | 195 |
| 2 | 1318 | 496.23 | 41.97 | 2 | 991.45 | QINDYVEK | 1.30 | P < 0.005 | 203 |
| 2 | 942 | 445.93 | 24.24 | 4 | 1780.70 | TDTSHHDQDHPTFNK | 1.30 | P < 0.005 | 194 |
| 2 | 572 | 402.22 | 59.19 | 3 | 1204.64 | KLSSWVLLMK | 1.29 | P < 0.001 | 205 |
| 2 | 1320 | 496.72 | 40.68 | 2 | 992.43 | QINDYVEK | 1.29 | P < 0.05 | 203 |
| 2 | 1621 | 540.25 | 28.98 | 2 | 1079.49 | FLENEDRR | 1.28 | P < 0.005 | 204 |
| 2 | 402 | 379.85 | 26.44 | 3 | 1137.53 | KQINDYVEK | 1.28 | P < 0.005 | 193 |
| 2 | 1617 | 539.75 | 26.15 | 2 | 1078.49 | FLENEDRR | 1.27 | P < 0.001 | 204 |
| 2 | 1383 | 505.24 | 30.47 | 2 | 1009.47 | QINDYVEK | 1.27 | P < 0.05 | 203 |
| 2 | 1806 | 568.79 | 28.87 | 2 | 1136.57 | KQINDYVEK | 1.27 | P < 0.005 | 193 |
| 2 | 2649 | 753.70 | 80.23 | 3 | 2259.08 | GTEAAGAMFLEAIPMSIPPEVK | 1.26 | P < 0.05 | 201 |
| 2 | 1321 | 496.73 | 43.77 | 2 | 992.45 | QINDYVEK | 1.26 | P < 0.01 | 203 |
| 2 | 3312 | 508.30 | 52.51 | 2 | 1015.59 | SVLGQLGITK | 1.26 | P < 0.001 | 192 |
| 2 | 1786 | 565.53 | 80.28 | 4 | 2259.10 | GTEAAGAMFLEAIPMSIPPEVK | 1.25 | P < 0.01 | 201 |
| 2 | 11 | 303.15 | 25.22 | 2 | 605.29 | VPMMK | 1.25 | P < 0.001 | 208 |
| 2 | 398 | 379.52 | 28.88 | 3 | 1136.54 | KQINDYVEK | 1.25 | P < 0.005 | 191 |
| 2 | 4557 | 301.92 | 59.04 | 4 | 1204.66 | KLSSWVLLMK | 1.24 | CountDiff | 205 |
| 2 | 255 | 353.48 | 14.49 | 3 | 1058.42 | EDPQGDAAQK | 1.23 | P < 0.05 | 200 |
| 2 | 1807 | 569.28 | 26.46 | 2 | 1137.55 | KQINDYVEK | 1.23 | P < 0.005 | 191 |
| 2 | 315 | 364.18 | 40.78 | 3 | 1090.52 | WERPFEVK | 1.23 | P < 0.05 | 198 |
| 2 | 3492 | 611.96 | 47.74 | 3 | 1833.86 | VFSNGADLSGVTEEAPLK | 1.21 | P < 0.05 | 190 |
| 2 | 3658 | 686.64 | 59.50 | 3 | 2057.90 | LYHSEAFTVNFGDTEEAK | 1.21 | P < 0.05 | 189 |
| 2 | 1670 | 547.94 | 80.71 | 3 | 1641.80 | ITPNLAEFAFSLYR | 1.21 | P < 0.01 | 188 |
| 2 | 2636 | 750.39 | 34.17 | 1 | 750.39 | FLEDVK | 1.21 | P < 0.05 | 187 |
| 2 | 4135 | 1204.70 | 59.07 | 1 | 1204.70 | KLSSWVLLMK | 1.20 | CountDiff | 205 |
| 2 | 1620 | 540.25 | 27.54 | 2 | 1079.49 | FLENEDRR | 1.19 | P < 0.05 | 204 |
| 2 | 3229 | 459.47 | 57.79 | 4 | 1834.86 | VFSNGADLSGVTEEAPLK | 1.17 | P < 0.01 | 190 |
| 2 | 1968 | 593.91 | 23.40 | 3 | 1779.71 | TDTSHHDQDHPTFNK | 1.17 | P < 0.05 | 194 |
| 2 | 272 | 356.94 | 24.24 | 5 | 1780.67 | TDTSHHDQDHPTFNK | 1.16 | P < 0.05 | 194 |
| 2 | 294 | 360.16 | 26.16 | 3 | 1078.46 | FLENEDRR | 1.16 | P < 0.005 | 204 |
| 2 | 2139 | 624.78 | 46.12 | 2 | 1248.55 | LGMFNIQHCK | 1.16 | P < 0.01 | 39 |
| 2 | 156 | 336.83 | 31.34 | 3 | 1008.47 | QINDYVEK | 1.15 | P < 0.05 | 203 |
| 2 | 1380 | 504.73 | 31.34 | 2 | 1008.45 | QINDYVEK | 1.10 | P < 0.01 | 186 |
| 2 | 675 | 416.84 | 46.11 | 3 | 1248.50 | LGMFNIQHCK | 1.09 | P < 0.005 | 39 |
| 2 | 491 | 390.19 | 37.54 | 2 | 779.37 | SPLFMGK | 1.09 | P < 0.05 | 195 |
| 2 | 929 | 444.72 | 35.91 | 2 | 888.43 | AVLTIDEK | 1.08 | P < 0.05 | 199 |
| 2 | 3461 | 594.25 | 24.23 | 3 | 1780.73 | TDTSHHDQDHPTFNK | 1.06 | CountDiff | 194 |
| 3 | 3420 | 570.29 | 33.93 | 3 | 1708.85 | LRTEGDGVYTLNDKK | 2.53 | P < 0.005 | 235 |
| 3 | 3071 | 1117.82 | 53.91 | 3 | 3351.44 | VDSGNDVTDIADDGCPKPPEIAHGYVEHSVR | 2.34 | P < 0.001 | 234 |
| 3 | 769 | 427.97 | 33.90 | 4 | 1708.86 | LRTEGDGVYTLNDKK | 2.28 | P < 0.005 | 235 |
| 3 | 2999 | 988.76 | 48.87 | 3 | 2964.26 | LPECEADDGCPKPPEIAHGYVEHSVR | 2.25 | P < 0.001 | 233 |
| 3 | 1967 | 593.65 | 48.19 | 5 | 2964.22 | LPECEADDGCPKPPEIAHGYVEHSVR | 2.18 | P < 0.05 | 233 |
| 3 | 1819 | 570.28 | 37.85 | 3 | 1708.82 | LRTEGDGVYTLNDKK | 2.15 | P < 0.001 | 235 |
| 3 | 2609 | 741.82 | 48.89 | 4 | 2964.26 | LPECEADDGCPKPPEIAHGYVEHSVR | 2.04 | P < 0.001 | 233 |
| 3 | 2544 | 720.84 | 33.43 | 2 | 1440.67 | TEGDGVYTLNNEK | 2.00 | P < 0.005 | 41 |
| 3 | 1965 | 593.66 | 48.80 | 5 | 2964.27 | LPECEADDGCPKPPEIAHGYVEHSVR | 1.99 | P < 0.005 | 233 |
| 3 | 2871 | 859.37 | 50.10 | 4 | 3434.46 | AVGDKLPECEADDGCPKPPEIAHGYVEHSVR | 1.89 | P < 0.001 | 178 |
| 3 | 2543 | 720.82 | 37.19 | 2 | 1440.63 | TEGDGVYTLNNEK | 1.87 | P < 0.001 | 41 |
| 3 | 2861 | 854.92 | 37.84 | 2 | 1708.83 | LRTEGDGVYTLNDKK | 1.82 | P < 0.005 | 235 |
| 3 | 1932 | 587.27 | 9.84 | 1 | 587.27 | NYYK | 1.81 | P < 0.05 | 232 |
| 3 | 2939 | 923.52 | 29.90 | 1 | 923.52 | ILGGHLDAK | 1.80 | P < 0.001 | 231 |
| 3 | 2498 | 709.90 | 54.12 | 2 | 1418.79 | DIAPTLTLYVGKK | 1.80 | P < 0.005 | 230 |
| 3 | 2995 | 980.48 | 41.32 | 1 | 980.48 | VGYVSGWGR | 1.76 | P < 0.001 | 229 |
| 3 | 2833 | 838.61 | 53.60 | 4 | 3351.42 | VDSGNDVTDIADDGCPKPPEIAHGYVEHSVR | 1.74 | P < 0.05 | 234 |
| 3 | 2945 | 930.43 | 41.11 | 2 | 1859.85 | AVGDKLPECEAVCGKPK | 1.72 | P < 0.001 | 228 |
| 3 | 1160 | 473.60 | 54.15 | 3 | 1418.78 | DIAPTLTLYVGKK | 1.70 | P < 0.005 | 230 |
| 3 | 3096 | 1203.63 | 48.75 | 1 | 1203.63 | VTSIQDWVQK | 1.67 | P < 0.005 | 227 |
| 3 | 2564 | 725.33 | 60.53 | 3 | 2173.97 | SPVGVQPILNEHTFCAGMSK | 1.67 | P < 0.001 | 226 |
| 3 | 2409 | 687.70 | 50.10 | 5 | 3434.47 | AVGDKLPECEADDGCPKPPEIAHGYVEHSVR | 1.65 | P < 0.001 | 178 |
| 3 | 1218 | 480.89 | 33.41 | 3 | 1440.65 | TEGDGVYTLNNEK | 1.62 | P < 0.01 | 41 |
| 3 | 973 | 449.54 | 49.09 | 3 | 1346.60 | SCAVAEYGVYVK | 1.60 | P < 0.05 | 225 |
| 3 | 2772 | 809.37 | 26.61 | 1 | 809.37 | DYAEVGR | 1.60 | P < 0.01 | 224 |
| 3 | 2595 | 739.35 | 25.15 | 1 | 739.35 | NPANPVQ | 1.59 | P < 0.005 | 223 |
| 3 | 2473 | 703.36 | 20.10 | 1 | 703.36 | VSVNER | 1.58 | P < 0.05 | 222 |
| 3 | 1212 | 480.55 | 35.92 | 3 | 1439.63 | TEGDGVYTLNNEK | 1.58 | P < 0.005 | 41 |
| 3 | 2841 | 841.45 | 47.55 | 1 | 841.45 | QLVEIEK | 1.56 | P < 0.005 | 221 |
| 3 | 2365 | 673.80 | 49.03 | 2 | 1346.59 | SCAVAEYGVYVK | 1.54 | P < 0.01 | 225 |
| 3 | 1069 | 462.26 | 29.89 | 2 | 923.51 | ILGGHLDAK | 1.51 | P < 0.001 | 231 |
| 3 | 709 | 421.22 | 47.56 | 2 | 841.43 | QLVEIEK | 1.49 | P < 0.005 | 221 |
| 3 | 1646 | 544.00 | 58.85 | 4 | 2172.98 | SPVGVQPILNEHTFCAGMSK | 1.49 | P < 0.005 | 226 |
| 3 | 2539 | 720.31 | 35.92 | 2 | 1439.61 | TEGDGVYTLNNEK | 1.48 | P < 0.005 | 41 |
| 3 | 2541 | 720.34 | 31.82 | 2 | 1439.67 | TEGDGVYTLNDKK | 1.47 | P < 0.005 | 220 |
| 3 | 2232 | 637.81 | 20.33 | 2 | 1274.61 | HYEGSTVPEKK | 1.42 | P < 0.05 | 219 |
| 3 | 2985 | 969.63 | 88.71 | 4 | 3875.50 | YQEDTCYGDAGSAFAVHDLEEDTWYAT- | 1.41 | P < 0.05 | 218 |
| | | | | | | GILSFDK | | | |
| 3 | 2355 | 671.09 | 53.87 | 5 | 3351.42 | VDSGNDVTDIADDGCPKPPEIAHGYVEHSVR | 1.41 | P < 0.01 | 234 |
| 3 | 3059 | 1087.01 | 58.86 | 2 | 2173.01 | SPVGVQPILNEHTFCAGMSK | 1.40 | P < 0.05 | 226 |
| 3 | 3116 | 1290.72 | 60.13 | 1 | 1290.72 | DIAPTLTLYVGK | 1.39 | P < 0.05 | 217 |
| 3 | 2312 | 656.29 | 36.35 | 2 | 1311.57 | TEGDGVYTLNDK | 1.38 | P < 0.05 | 216 |
| 3 | 1850 | 573.76 | 22.69 | 2 | 1146.51 | HYEGSTVPEK | 1.36 | P < 0.05 | 215 |
| 3 | 139 | 333.17 | 41.26 | 3 | 997.49 | HTFCAGMSK | 1.36 | P < 0.05 | 214 |
| 3 | 3123 | 1346.62 | 48.94 | 1 | 1346.62 | SCAVAEYGVYVK | 1.33 | P < 0.05 | 225 |
| 3 | 612 | 408.74 | 20.77 | 2 | 816.47 | KQWINK | 1.33 | P < 0.05 | 213 |
| 3 | 565 | 401.87 | 48.75 | 3 | 1203.59 | VTSIQDWVQK | 1.32 | P < 0.05 | 227 |
| 3 | 2123 | 620.62 | 41.12 | 3 | 1859.84 | AVGDKLPECEAVCGKPK | 1.31 | P < 0.001 | 228 |
| 3 | 802 | 430.90 | 60.12 | 3 | 1290.68 | DIAPTLTLYVGK | 1.29 | P < 0.05 | 217 |
| 3 | 110 | 327.49 | 41.31 | 3 | 980.45 | VGYVSGWGR | 1.27 | P < 0.05 | 229 |
| 3 | 247 | 352.18 | 20.09 | 2 | 703.35 | VSVNER | 1.26 | P < 0.05 | 212 |
| 3 | 2562 | 724.99 | 58.85 | 3 | 2172.95 | SPVGVQPILNEHTFCAGMSK | 1.23 | P < 0.05 | 226 |
| 3 | 1086 | 465.71 | 41.11 | 4 | 1859.82 | AVGDKLPECEAVCGKPK | 1.23 | P < 0.05 | 228 |
| 3 | 2266 | 645.85 | 60.13 | 2 | 1290.69 | DIAPTLTLYVGK | 1.22 | P < 0.05 | 217 |
| 3 | 29 | 308.50 | 29.88 | 3 | 923.48 | ILGGHLDAK | 1.19 | P < 0.05 | 231 |
| 3 | 1284 | 490.72 | 41.32 | 2 | 980.43 | VGYVSGWGR | 1.17 | P < 0.01 | 229 |
| 3 | 4748 | 1311.60 | 36.30 | 1 | 1311.60 | TEGDGVYTLNDK | 1.16 | CountDiff | 216 |
| 3 | 2028 | 602.29 | 48.75 | 2 | 1203.57 | VTSIQDWVQK | 1.15 | P < 0.05 | 227 |
| 4 | 1140 | 471.74 | 26.99 | 2 | 942.47 | YTVNQCR | 1.94 | P < 0.05 | 258 |
| 4 | 1561 | 529.91 | 39.96 | 3 | 1587.71 | RQSEDSTFYLGER | 1.57 | P < 0.001 | 257 |
| 4 | 2643 | 752.71 | 48.24 | 3 | 2256.11 | KAEEEHLGILGPQLHADVGDK | 1.52 | P < 0.01 | 266 |
| 4 | 67 | 316.67 | 22.82 | 2 | 632.33 | VFNPR | 1.48 | P < 0.005 | 255 |
| 4 | 2401 | 686.37 | 58.92 | 2 | 1371.73 | GAYPLSIEPIGVR | 1.47 | P < 0.005 | 254 |
| 4 | 2729 | 788.91 | 79.06 | 2 | 1576.81 | DLYSGLIGPLIVCR | 1.45 | P < 0.01 | 253 |
| 4 | 2584 | 735.96 | 56.77 | 3 | 2205.86 | MHSMNGFMYGNQPGLTMCK | 1.45 | P < 0.05 | 252 |
| 4 | 1864 | 575.78 | 49.78 | 4 | 2300.10 | KLISVDTEHSNIYLQNGPDR | 1.43 | P < 0.005 | 251 |
| 4 | 2748 | 794.36 | 39.93 | 2 | 1587.71 | RQSEDSTFYLGER | 1.41 | P < 0.05 | 257 |
| 4 | 2026 | 602.26 | 35.20 | 2 | 1203.51 | EYTDASFTNR | 1.41 | P < 0.01 | 250 |
| 4 | 2809 | 829.75 | 82.88 | 3 | 2487.23 | GPEEEHLGILGPVIWAEVGDTIR | 1.39 | P < 0.05 | 249 |
| 4 | 2013 | 600.27 | 68.78 | 4 | 2398.06 | HYYIGIIETTWDYASDHGEK | 1.39 | CountDiff | 248 |
| 4 | 3802 | 767.38 | 49.80 | 3 | 2300.12 | KLISVDTEHSNIYLQNGPDR | 1.38 | P < 0.01 | 251 |
| 4 | 1780 | 564.78 | 48.24 | 4 | 2256.10 | KAEEEHLGILGPQLHADVGDK | 1.37 | P < 0.05 | 266 |
| 4 | 1934 | 587.77 | 39.28 | 2 | 1174.53 | MYYSAVDPTK | 1.36 | P < 0.01 | 247 |
| 4 | 1401 | 509.22 | 31.00 | 2 | 1017.43 | QYTDSTFR | 1.36 | P < 0.005 | 246 |
| 4 | 1536 | 526.27 | 79.07 | 3 | 1576.79 | DLYSGLIGPLIVCR | 1.35 | P < 0.05 | 253 |
| 4 | 2666 | 760.36 | 57.82 | 2 | 1519.71 | ALYLQYTDETFR | 1.35 | P < 0.05 | 245 |
| 4 | 2705 | 775.36 | 63.08 | 5 | 3872.77 | NMATRPYSI- | 1.35 | P < 0.05 | 224 |
| | | | | | | HAHGVQTESSTVTPTLPGETLTYVWK | | | |
| 4 | 2558 | 724.35 | 51.55 | 3 | 2171.03 | LISVDTEHSNIYLQNGPDR | 1.35 | P < 0.05 | 243 |
| 4 | 2583 | 735.90 | 75.75 | 2 | 1470.79 | DIASGLIGPLIICK | 1.34 | P < 0.05 | 242 |
| 4 | 1286 | 490.93 | 75.75 | 3 | 1470.77 | DIASGLIGPLIICK | 1.34 | P < 0.05 | 242 |
| 4 | 2586 | 736.34 | 45.93 | 2 | 1471.67 | EVGPTNADPVCLAK | 1.33 | P < 0.005 | 241 |
| 4 | 1397 | 507.70 | 24.45 | 2 | 1014.39 | TYCSEPEK | 1.33 | P < 0.05 | 240 |
| 4 | 512 | 394.20 | 26.12 | 3 | 1180.58 | IYHSHIDAPK | 1.32 | P < 0.01 | 239 |
| 4 | 1135 | 471.20 | 25.73 | 2 | 941.39 | YTVNQCR | 1.30 | P < 0.005 | 258 |
| 4 | 1573 | 532.56 | 26.31 | 3 | 1595.66 | VDKDNEDFQESNR | 1.30 | P < 0.05 | 238 |
| 4 | 1574 | 532.76 | 51.91 | 4 | 2128.02 | AEEEHLGILGPQLHADVGDK | 1.28 | P < 0.05 | 237 |
| 4 | 2499 | 710.02 | 51.91 | 3 | 2128.04 | AEEEHLGILGPQLHADVGDK | 1.26 | P < 0.05 | 237 |
| 4 | 1862 | 575.55 | 48.94 | 4 | 2299.18 | KLISVDTEHSNIYLQNGPDR | 1.14 | CountDiff | 251 |
| 4 | 3929 | 854.90 | 82.74 | 4 | 3416.58 | QKDVDKEFYLFPTVFDENESLLLEDNIR | 1.10 | CountDiff | 236 |
| 5 | 2667 | 760.39 | 23.11 | 1 | 760.39 | VDSHFR | 1.60 | P < 0.001 | 259 |
| 5 | 409 | 380.69 | 23.09 | 2 | 760.37 | VDSHFR | 1.35 | P < 0.05 | 259 |
| 6 | 1774 | 564.27 | 58.75 | 3 | 1690.79 | VFDEFKPLVEEPQN | 1.48 | P < 0.05 | 263 |
| 6 | 3016 | 1000.59 | 48.12 | 1 | 1000.59 | QTALVELVK | 1.46 | P < 0.05 | 262 |
| 6 | 293 | 359.82 | 26.81 | 3 | 1077.44 | NECFLQHK | 1.30 | CountDiff | 261 |
| 6 | 4758 | 567.28 | 85.77 | 3 | 1699.82 | RHPYFYAPELLFF | 1.11 | CountDiff | 260 |
| 7 | 2684 | 766.36 | 34.14 | 1 | 766.36 | DSLEFR | 2.03 | P < 0.001 | 275 |
| 7 | 3139 | 331.85 | 34.23 | 3 | 993.53 | KLINDYVK | 1.74 | CountDiff | 274 |
| 7 | 436 | 383.68 | 34.14 | 2 | 766.35 | DSLEFR | 1.74 | P < 0.001 | 275 |
| 7 | 3797 | 766.05 | 95.21 | 3 | 2296.13 | DYNLNDILLQLGIEEAFTSK | 1.72 | P < 0.005 | 273 |
| 7 | 2184 | 631.28 | 57.32 | 3 | 1891.82 | LYGSEAFATDFQDSAAAK | 1.66 | P < 0.005 | 272 |
| 7 | 2898 | 887.11 | 86.85 | 3 | 2659.31 | FNRPFLMIIVPTDTQNIFFMSK | 1.65 | P < 0.05 | 271 |
| 7 | 129 | 331.52 | 34.08 | 3 | 992.54 | KLINDYVK | 1.60 | P < 0.01 | 274 |
| 7 | 3623 | 665.58 | 86.85 | 4 | 2659.30 | FNRPFLMIIVPTDTQNIFFMSK | 1.58 | P < 0.05 | 271 |
| 7 | 2968 | 946.43 | 57.21 | 2 | 1891.85 | LYGSEAFATDFQDSAAAK | 1.55 | CountDiff | 272 |
| 7 | 2508 | 711.82 | 54.99 | 2 | 1422.63 | DEELSCTVVELK | 1.55 | P < 0.005 | 270 |
| 7 | 1322 | 496.78 | 34.08 | 2 | 992.55 | KLINDYVK | 1.53 | P < 0.005 | 274 |
| 7 | 594 | 405.90 | 69.11 | 3 | 1215.68 | ITLLSALVETR | 1.52 | P < 0.05 | 269 |
| 7 | 3544 | 631.62 | 59.32 | 3 | 1892.84 | LYGSEAFATDFQDSAAAK | 1.48 | P < 0.01 | 272 |
| 7 | 1214 | 480.75 | 37.54 | 2 | 960.49 | ADLSGITGAR | 1.47 | P < 0.01 | 264 |
| 7 | 323 | 365.54 | 34.44 | 3 | 1094.60 | NLAVSQVVHK | 1.46 | P < 0.01 | 267 |
| 7 | 1984 | 596.97 | 63.89 | 3 | 1788.89 | MEEVEAMLLPETLKR | 1.45 | P < 0.01 | 266 |
| 7 | 2061 | 608.35 | 69.09 | 2 | 1215.69 | ITLLSALVETR | 1.43 | P < 0.01 | 269 |
| 7 | 1668 | 547.81 | 34.44 | 2 | 1094.61 | NLAVSQVVHK | 1.40 | P < 0.05 | 267 |
| 7 | 2979 | 954.47 | 63.24 | 2 | 1907.93 | AVLDVFEEGTEASAATAVK | 1.36 | P < 0.05 | 265 |
| 7 | 4027 | 960.50 | 37.56 | 1 | 960.50 | ADLSGITGAR | 1.35 | CountDiff | 264 |
| 8 | 1438 | 514.27 | 39.16 | 2 | 1027.53 | TEVNVLPGAK | 1.31 | CountDiff | 86 |
| 9 | 2191 | 631.99 | 78.41 | 3 | 1893.95 | ENQLEVLEVSWLHGLK | 1.92 | P < 0.001 | 286 |
| 9 | 671 | 416.69 | 23.35 | 2 | 832.37 | CAGPEAVK | 1.78 | P < 0.001 | 285 |
| 9 | 445 | 384.86 | 38.38 | 3 | 1152.56 | ALGHLDLSGNR | 1.75 | P < 0.001 | 284 |
| 9 | 1950 | 590.33 | 58.67 | 2 | 1179.65 | DLLLPQPDLR | 1.72 | P < 0.001 | 283 |
| 9 | 2383 | 679.68 | 72.68 | 3 | 2037.02 | TLDLGENQLETLPPDLLR | 1.70 | P < 0.001 | 282 |
| 9 | 2607 | 740.88 | 74.39 | 4 | 2960.50 | LQELHLSSNGLESLSPEFLRPVPQLR | 1.70 | P < 0.005 | 281 |
| 9 | 2808 | 829.35 | 78.18 | 3 | 2486.03 | DGFDISGNPWICDQNLSDLYR | 1.67 | P < 0.01 | 280 |
| 9 | 600 | 406.72 | 34.62 | 2 | 812.43 | GPLQLER | 1.62 | P < 0.001 | 279 |
| 9 | 985 | 450.77 | 38.00 | 2 | 900.53 | GQTLLAVAK | 1.58 | P < 0.05 | 278 |
| 9 | 646 | 413.17 | 32.12 | 2 | 825.33 | DCQVFR | 1.55 | P < 0.001 | 111 |
| 9 | 4677 | 740.64 | 73.67 | 4 | 2959.54 | LQELHLSSNGLESLSPEFLRPVPQLR | 1.51 | CountDiff | 281 |
| 9 | 4592 | 576.79 | 38.33 | 2 | 1152.57 | ALGHLDLSGNR | 1.49 | P < 0.05 | 284 |
| 9 | 2712 | 780.76 | 89.68 | 3 | 2340.26 | NALTGLPPGLFQASATLDTLVLK | 1.46 | P < 0.05 | 276 |
| 9 | 4728 | 1019.02 | 72.69 | 2 | 2037.03 | TLDLGENQLETLPPDLLR | 1.37 | P < 0.05 | 282 |
| 9 | 4771 | 947.48 | 78.41 | 2 | 1893.95 | ENQLEVLEVSWLHGLK | 1.35 | CountDiff | 286 |
| 9 | 2915 | 900.44 | 37.14 | 1 | 900.44 | GQTLLAVAK | 1.32 | P < 0.05 | 278 |
| 9 | 511 | 393.89 | 58.68 | 3 | 1179.65 | DLLLPQPDLR | 1.24 | CountDiff | 283 |
| 9 | 3220 | 450.75 | 39.00 | 2 | 900.49 | GQTLLAVAK | 1.16 | CountDiff | 278 |
| 9 | 4778 | 1243.52 | 78.23 | 2 | 2486.03 | DGFDISGNPWICDQNLSDLYR | 1.11 | CountDiff | 280 |
| 10 | 2870 | 859.12 | 79.89 | 4 | 3433.46 | NWGLSVYADKPETTKEQLGEFYEALDC*LR | 2.16 | P < 0.05 | 295 |
| 10 | 654 | 414.20 | 37.69 | 3 | 1240.58 | SDVVYTDWKK | 1.89 | P < 0.01 | 294 |
| 10 | 4066 | 1019.45 | 23.19 | 1 | 1019.45 | DKCEPLEK | 1.63 | CountDiff | 293 |
| 10 | 3514 | 620.80 | 37.67 | 2 | 1240.59 | SDVVYTDWKK | 1.63 | P < 0.005 | 294 |
| 10 | 2554 | 723.32 | 59.69 | 2 | 1445.63 | TYMLAFDVNDEK | 1.55 | P < 0.05 | 292 |
| 10 | 2720 | 784.61 | 69.90 | 4 | 3135.42 | TYMLAFDVNDEKNWGLSVYADKPETTK | 1.51 | P < 0.001 | 291 |
| 10 | 1229 | 482.55 | 57.42 | 3 | 1445.63 | TYMLAFDVNDEK | 1.49 | P < 0.001 | 292 |
| 10 | 429 | 383.18 | 15.73 | 3 | 1147.52 | KDKCEPLEK | 1.48 | P < 0.005 | 290 |
| 10 | 2553 | 723.31 | 57.42 | 2 | 1445.61 | TYMLAFDVNDEK | 1.46 | P < 0.001 | 292 |
| 10 | 2706 | 776.34 | 23.12 | 1 | 776.34 | CEPLEK | 1.45 | P < 0.005 | 289 |
| 10 | 465 | 387.52 | 67.40 | 3 | 1160.54 | WFYIASAFR | 1.43 | P < 0.001 | 97 |
| 10 | 3086 | 1160.57 | 67.41 | 1 | 1160.57 | WFYIASAFR | 1.40 | P < 0.01 | 97 |
| 10 | 1408 | 510.23 | 23.19 | 2 | 1019.45 | DKCEPLEK | 1.39 | P < 0.001 | 293 |
| 10 | 3428 | 574.28 | 15.72 | 2 | 1147.55 | KDKCEPLEK | 1.34 | P < 0.05 | 288 |
| 10 | 2882 | 872.36 | 73.90 | 2 | 1743.71 | EQLGEFYEALDCLR | 1.33 | P < 0.005 | 287 |
| 10 | 1901 | 581.92 | 73.90 | 3 | 1743.74 | EQLGEFYEALDCLR | 1.32 | P < 0.001 | 287 |
| 10 | 3067 | 1112.51 | 43.25 | 1 | 1112.51 | SDVVYTDWK | 1.29 | P < 0.05 | 294 |
| 10 | 180 | 340.48 | 23.19 | 3 | 1019.42 | DKCEPLEK | 1.29 | P < 0.01 | 293 |
| 10 | 479 | 388.66 | 23.11 | 2 | 776.31 | CEPLEK | 1.21 | P < 0.05 | 289 |
| 10 | 1895 | 580.77 | 67.40 | 2 | 1160.53 | WFYIASAFR | 1.17 | P < 0.005 | 97 |
| 10 | 4079 | 1045.81 | 69.87 | 3 | 3135.41 | TYMLAFDVNDEKNWGLSVYADKPETTK | 1.16 | CountDiff | 291 |
| 11 | 1744 | 560.26 | 55.53 | 3 | 1678.76 | FNWYVDGVEVHNAK | 1.79 | P < 0.05 | 300 |
| 11 | 4157 | 1322.65 | 48.52 | 1 | 1322.65 | STSGGTAALGCLVK | 1.68 | P < 0.01 | 299 |
| 11 | 2835 | 839.38 | 54.16 | 2 | 1677.75 | FNWYVDGVEVHNAK | 1.68 | P < 0.005 | 300 |
| 11 | 1593 | 535.74 | 59.48 | 4 | 2139.94 | TPEVTCVVVDVSHEDPEVK | 1.62 | P < 0.05 | 198 |
| 11 | 1740 | 559.92 | 54.07 | 3 | 1677.74 | FNWYVDGVEVHNAK | 1.61 | P < 0.005 | 300 |
| 11 | 2340 | 668.30 | 71.35 | 5 | 3337.47 | SCDKTHTCPPCPAPELLGGPSVFLFPPKPK | 1.56 | P < 0.05 | 297 |
| 11 | 1743 | 560.26 | 56.82 | 3 | 1678.76 | FNWYVDGVEVHNAK | 1.54 | P < 0.05 | 300 |
| 11 | 899 | 441.55 | 48.52 | 3 | 1322.63 | STSGGTAALGCLVK | 1.49 | P < 0.05 | 299 |
| 11 | 2523 | 713.99 | 59.48 | 3 | 2139.95 | TPEVTCVVVDVSHEDPEVK | 1.35 | P < 0.05 | 198 |
| 11 | 2820 | 835.42 | 35.66 | 1 | 835.42 | DTLMISR | 1.33 | P < 0.05 | 296 |
| 11 | 2328 | 661.81 | 48.52 | 2 | 1322.61 | STSGGTAALGCLVK | 1.33 | P < 0.05 | 299 |
| 12 | 1860 | 575.33 | 36.62 | 2 | 1149.65 | VVAGVANALAHK | 1.00 | CountDiff | 99 |
| 13 | 4206 | 385.22 | 29.34 | 2 | 769.43 | VVPEGIR | 1.44 | P < 0.01 | 302 |
| 13 | 2049 | 606.29 | 64.08 | 3 | 1816.85 | SNLDEDIIAEENIVSR | 1.17 | P < 0.05 | 301 |
| 14 | 1601 | 538.00 | 61.93 | 4 | 2148.98 | IFFHLNAVALGDGGHYTCR | 1.46 | P < 0.01 | 306 |
| 14 | 808 | 431.73 | 47.11 | 4 | 1723.90 | LELHVDGPPPRPQLR | 1.41 | P < 0.01 | 305 |
| 14 | 4039 | 987.48 | 39.95 | 1 | 987.48 | CLAPLEGAR | 1.38 | P < 0.05 | 304 |
| 14 | 2529 | 717.00 | 61.93 | 3 | 2148.98 | IFFHLNAVALGDGGHYTCR | 1.37 | P < 0.01 | 306 |
| 14 | 1309 | 494.24 | 39.92 | 2 | 987.47 | CLAPLEGAR | 1.22 | P < 0.05 | 304 |
| 14 | 12 | 303.18 | 35.81 | 2 | 605.35 | FALVR | 1.18 | P < 0.05 | 303 |
| 16 | 250 | 352.67 | 21.82 | 2 | 704.33 | VEIDTK | 1.61 | P < 0.05 | 307 |
| 16 | 1018 | 456.25 | 59.04 | 3 | 1366.73 | GSPAINVAVHVFR | 1.39 | P < 0.005 | 115 |
| 18 | 1680 | 549.94 | 53.10 | 3 | 1647.80 | KALYLQYTDETFR | 1.53 | P < 0.05 | 311 |
| 18 | 2276 | 648.51 | 38.14 | 4 | 2591.02 | TYC*SEPEKVDKDNEDFQESNR | 1.49 | P < 0.005 | 310 |
| 18 | 4103 | 1089.12 | 72.07 | 3 | 3265.34 | VYPGEQYTYMLLATEEQSPGEGDGNC*VTR | 1.35 | P < 0.05 | 309 |
| 18 | 2086 | 613.26 | 37.16 | 2 | 1225.51 | DDEEFIESNK | 1.25 | P < 0.05 | 308 |
| 19 | 1375 | 504.28 | 41.91 | 2 | 1007.55 | IYISGMAPR | 1.53 | P < 0.05 | 313 |
| 19 | 1709 | 556.27 | 52.56 | 3 | 1666.79 | DWHGVPGQVDAAMAGR | 1.48 | P < 0.01 | 312 |
| 19 | 2304 | 653.79 | 36.97 | 2 | 1306.57 | GQYC*YELDEK | 1.37 | P < 0.005 | 124 |
| 19 | 2301 | 653.26 | 40.78 | 2 | 1305.51 | GQYC*YELDEK | 1.36 | P < 0.05 | 124 |
| 19 | 852 | 436.19 | 36.96 | 3 | 1306.55 | GQYC*YELDEK | 1.32 | P < 0.01 | 124 |
| 19 | 109 | 327.39 | 36.97 | 4 | 1306.54 | GQYC*YELDEK | 1.32 | P < 0.01 | 124 |
| 19 | 4520 | 833.88 | 52.52 | 2 | 1666.75 | DWHGVPGQVDAAMAGR | 1.20 | CountDiff | 312 |
| 20 | 451 | 386.19 | 44.02 | 3 | 1156.55 | SPAFTDLHLR | 1.37 | P < 0.005 | 315 |
| 20 | 453 | 386.23 | 50.27 | 3 | 1156.67 | SPAFTDLHLR | 1.31 | P < 0.05 | 315 |
| 20 | 317 | 364.23 | 34.28 | 2 | 727.45 | LAIPEGK | 1.28 | P < 0.005 | 314 |
| 21 | 2928 | 918.44 | 56.38 | 2 | 1835.87 | QEPSQGTTTFAVTSILR | 1.60 | P < 0.005 | 319 |
| 21 | 4136 | 1213.62 | 44.11 | 1 | 1213.62 | WLQGSQELPR | 1.45 | P < 0.05 | 318 |
| 21 | 2083 | 612.62 | 56.39 | 3 | 1835.84 | QEPSQGTTTFAVTSILR | 1.40 | P < 0.005 | 317 |
| 21 | 2785 | 818.39 | 28.77 | 1 | 818.39 | VAAEDWK | 1.38 | P < 0.05 | 316 |
| 22 | 3272 | 489.25 | 45.65 | 3 | 1465.73 | SKLIC*QATGFSPR | 1.68 | P < 0.05 | 322 |
| 22 | 3678 | 695.08 | 54.13 | 4 | 2777.30 | YAATSQVLLPSKDVMQGTDEHVVC*K | 1.50 | P < 0.05 | 321 |
| 22 | 684 | 417.84 | 44.18 | 3 | 1251.50 | LIC*QATGFSPR | 1.33 | P < 0.05 | 320 |
| 23 | 4130 | 1183.62 | 101.59 | 2 | 2366.23 | VPTADLEDVLPLAEDITNILSK | 1.34 | P < 0.05 | 325 |
| 23 | 2756 | 799.48 | 34.81 | 1 | 799.48 | VLEPTLK | 1.27 | P < 0.05 | 324 |
| 23 | 2456 | 699.25 | 52.46 | 3 | 2095.73 | SLGECCDVEDSTTCFNAK | 1.25 | P < 0.05 | 323 |
| 24 | 4723 | 998.50 | 66.65 | 2 | 1995.99 | LAVTTHGLPCLAWASAQAK | 1.12 | CountDiff | 326 |
| 24 | 2593 | 738.65 | 55.62 | 3 | 2213.93 | DKLAAC*LEGNC*AEGLGTNYR | 1.10 | CountDiff | 127 |
| 26 | 4781 | 516.26 | 31.65 | 2 | 1031.51 | LSPLGEEMR | 1.26 | CountDiff | 328 |
| 27 | 1892 | 580.03 | 74.20 | 4 | 2317.10 | QSNNKYAASSYLSLTPEQWK | 1.54 | P < 0.05 | 133 |
| 27 | 773 | 428.26 | 35.58 | 2 | 855.51 | LTVLGQPK | 1.33 | P < 0.05 | 329 |
| 27 | 4589 | 553.77 | 92.73 | 4 | 2212.06 | ATLVCLISDFYPGAVTVAWK | 1.16 | CountDiff | 328 |
| 27 | 3064 | 1106.55 | 92.76 | 2 | 2212.09 | ATLVCLISDFYPGAVTVAWK | 1.06 | CountDiff | 328 |
| 28 | 641 | 412.22 | 48.67 | 3 | 1234.64 | LCQDLGPGAFR | 1.40 | P < 0.01 | 330 |
| 29 | 232 | 349.72 | 34.08 | 2 | 698.43 | EVLLPK | 1.42 | P < 0.05 | 332 |
| 29 | 1087 | 465.74 | 55.72 | 2 | 930.47 | FAFNLYR | 1.30 | P < 0.05 | 331 |
| 30 | 3800 | 766.89 | 40.94 | 2 | 1532.77 | QKWEAEPVYVQR | 1.37 | P < 0.05 | 333 |
| 30 | 1752 | 561.24 | 39.93 | 2 | 1121.47 | QVEGMEDWK | 1.23 | P < 0.05 | 135 |
| 31 | 3080 | 1144.48 | 45.56 | 1 | 1144.48 | SDVMYTDWK | 1.77 | P < 0.005 | 334 |
| 31 | 1834 | 572.74 | 45.58 | 2 | 1144.47 | SDVMYTDWK | 1.46 | P < 0.005 | 334 |
| 31 | 3046 | 1057.95 | 81.30 | 2 | 2114.89 | EQLGEFYEALDCLCIPR | 1.35 | P < 0.05 | 136 |
| 31 | 2480 | 705.63 | 81.30 | 3 | 2114.87 | EQLGEFYEALDCLCIPR | 1.26 | P < 0.05 | 136 |
| 32 | 401 | 379.85 | 30.87 | 3 | 1137.53 | EQINNYVEK | 1.44 | P < 0.001 | 335 |
| 32 | 3418 | 569.28 | 30.85 | 2 | 1137.55 | EQINNYVEK | 1.34 | P < 0.005 | 335 |
| 33 | 4089 | 1068.47 | 40.91 | 2 | 2135.93 | VDNALQSGNSQESVTEQDSK | 1.72 | P < 0.001 | 337 |
| 33 | 2511 | 712.65 | 40.90 | 3 | 2135.93 | VDNALQSGNSQESVTEQDSK | 1.54 | P < 0.001 | 336 |
| 34 | 2675 | 762.36 | 62.95 | 1 | 762.36 | TQQRNN | 1.35 | P < 0.05 | 338 |
| 35 | 370 | 373.69 | 31.00 | 2 | 746.37 | LELSQR | 1.76 | P < 0.05 | 339 |
| 36 | 4306 | 493.78 | 30.24 | 2 | 986.55 | KQLVEIEK | 1.99 | P < 0.05 | 341 |
| 36 | 4514 | 790.89 | 38.17 | 2 | 1580.77 | LRTEGDGVYTLNDK | 1.98 | P < 0.01 | 340 |
| 36 | 1548 | 527.59 | 38.18 | 3 | 1580.75 | LRTEGDGVYTLNDK | 1.96 | P < 0.01 | 340 |
| 36 | 4336 | 527.92 | 39.48 | 3 | 1581.74 | LRTEGDGVYTLNDK | 1.63 | P < 0.05 | 340 |
| 36 | 120 | 329.52 | 30.18 | 3 | 986.54 | KQLVEIEK | 1.59 | P < 0.005 | 341 |
| 37 | 2976 | 952.40 | 39.38 | 2 | 1903.79 | NNEGTYYSPNYNPQSR | 1.45 | P < 0.01 | 343 |
| 37 | 2844 | 844.40 | 60.33 | 3 | 2531.18 | SVPPSASHVAPTETFTYEWTVPK | 1.41 | P < 0.01 | 342 |
| 37 | 2216 | 635.27 | 39.39 | 3 | 1903.79 | NNEGTYYSPNYNPQSR | 1.40 | P < 0.05 | 343 |
| 37 | 2201 | 633.55 | 60.33 | 4 | 2531.18 | SVPPSASHVAPTETFTYEWTVPK | 1.02 | CountDiff | 342 |
| 38 | 1661 | 546.64 | 84.80 | 3 | 1637.90 | VFAIPPSFASIFLTK | 1.55 | P < 0.05 | 344 |
| 39 | 3438 | 583.25 | 51.94 | 5 | 2912.22 | HYTNPSQDVTVPCPVPPPPPCCHPR | 1.88 | P < 0.05 | 345 |
| 41 | 1718 | 557.79 | 70.85 | 4 | 2228.14 | VVSVLTVLHQNWLDGKEYK | 2.34 | P < 0.001 | 346 |
| 42 | 2353 | 670.89 | 52.88 | 5 | 3350.42 | VDSGNDVTDIADDGCPKPPEIAHGYVEHSVR | 1.97 | P < 0.01 | 234 |
| 42 | 2354 | 670.89 | 53.18 | 5 | 3350.42 | VDSGNDVTDIADDGCPKPPEIAHGYVEHSVR | 1.26 | P < 0.05 | 234 |
| 43 | 2072 | 610.32 | 46.96 | 2 | 1219.63 | DFTCVHQALK | 1.61 | P < 0.005 | 347 |
| 44 | 1279 | 490.27 | 40.13 | 2 | 979.53 | LLIYGASSR | 1.47 | P < 0.05 | 348 |
| 45 | 711 | 421.57 | 50.27 | 3 | 1262.69 | KAACLDILMLR | 1.35 | P < 0.05 | 349 |
| 46 | 1677 | 549.60 | 77.21 | 3 | 1646.78 | DVWGIEGPIDAAFTR | 1.20 | P < 0.05 | 350 |
| 47 | 26 | 307.17 | 29.58 | 3 | 919.49 | EDTNKWK | 1.24 | P < 0.05 | 351 |
| 48 | 4695 | 463.73 | 23.52 | 2 | 926.45 | AVYDQSATA | 1.70 | P < 0.01 | 353 |
| 48 | 3596 | 655.36 | 43.34 | 3 | 1964.06 | ANAGKPKDPTFIPAPIQAK | 1.09 | CountDiff | 352 |
| 49 | 4479 | 684.35 | 77.89 | 2 | 1367.69 | DSSTWLTAFVLK | 1.18 | CountDiff | 354 |
| 50 | 1598 | 537.30 | 61.22 | 2 | 1073.59 | PMPVLLMGQA | 1.34 | P < 0.05 | 355 |
| 51 | 2394 | 683.33 | 52.43 | 3 | 2047.97 | RLYGSEAFATDFQDSAAAK | 2.19 | P < 0.01 | 146 |
| 55 | 800 | 430.81 | 56.63 | 5 | 2150.02 | SETEIHQGFQHLHQLFAK | 1.47 | P < 0.01 | 356 |
| 55 | 1603 | 538.25 | 64.49 | 4 | 2149.98 | SETEIHQGFQHLHQLFAK | 1.06 | CountDiff | 356 |
| 60 | 4760 | 579.78 | 62.41 | 2 | 1158.55 | KYFIDFVAR | 1.30 | CountDiff | 357 |
| 63 | 1822 | 570.61 | 39.19 | 3 | 1709.81 | EEPIPC*TAHWHFGQ | 1.70 | P < 0.05 | 358 |
| 64 | 1094 | 466.55 | 51.68 | 3 | 1397.63 | HNLKDAGEAEEGK | 1.66 | P < 0.05 | 359 |
| 65 | 332 | 366.42 | 33.97 | 4 | 1462.66 | GLSRTSMKPRSSR | 1.45 | P < 0.05 | 360 |
| 67 | 510 | 393.68 | 59.71 | 2 | 786.35 | DSSYMPS | 1.33 | P < 0.05 | 361 |
| 68 | 1912 | 583.37 | 41.93 | 1 | 583.37 | LPLIK | 1.55 | P < 0.001 | 362 |
| 69 | 2063 | 608.81 | 65.25 | 2 | 1216.61 | IAEFAFEYAR | 1.37 | P < 0.005 | 363 |
| 69 | 597 | 406.20 | 65.25 | 3 | 1216.58 | IAEFAFEYAR | 1.12 | CountDiff | 363 |
| 73 | 540 | 398.86 | 31.96 | 3 | 1194.56 | EGKLENGYRK | 1.39 | P < 0.005 | 364 |
| 76 | 2071 | 610.32 | 43.07 | 3 | 1828.94 | PQLDLFSCMLKHRLK | 1.40 | P < 0.05 | 365 |
| 77 | 2536 | 719.68 | 70.47 | 3 | 2157.02 | EAPTSLSQLLDNSGAPNVTIK | 1.26 | P < 0.05 | 366 |
| 78 | 4587 | 537.76 | 39.08 | 2 | 1074.51 | KVNEKDVDK | 1.45 | P < 0.05 | 367 |
| 79 | 1347 | 500.27 | 40.19 | 2 | 999.53 | AAYMNKER | 1.09 | CountDiff | 163 |
| 80 | 4074 | 1028.93 | 54.73 | 2 | 2056.85 | GQELC*ADYSENTFTEYK | 1.16 | P < 0.05 | 368 |
| 83 | 113 | 327.84 | 35.07 | 3 | 981.50 | KNGNVANYV | 2.61 | P < 0.01 | 369 |
| 83 | 112 | 327.84 | 34.08 | 3 | 981.50 | KNGNVANYV | 2.35 | P < 0.005 | 369 |
| 83 | 1283 | 490.71 | 32.04 | 2 | 980.41 | KNGNVANYV | 1.26 | P < 0.05 | 369 |
| 84 | 2497 | 709.81 | 59.62 | 2 | 1418.61 | M#PVINIEDLTEK | 1.29 | P < 0.05 | 370 |
| 85 | 198 | 343.53 | 54.20 | 3 | 1028.57 | LGKSVVAKVK | 1.38 | P < 0.005 | 371 |
| 87 | 885 | 439.74 | 29.59 | 2 | 878.47 | IM#KDVQK | 1.20 | P < 0.005 | 372 |
| 88 | 1412 | 510.30 | 54.39 | 2 | 1019.59 | EEAIAVTMR | 1.09 | CountDiff | 167 |
| 89 | 2127 | 621.31 | 55.34 | 3 | 1861.91 | ANPGYKWC*PTTNKPVK | 1.38 | P < 0.05 | 373 |
| 89 | 1089 | 466.23 | 53.39 | 4 | 1861.90 | ANPGYKWC*PTTNKPVK | 1.26 | P < 0.05 | 373 |
| 90 | 712 | 421.56 | 65.13 | 3 | 1262.66 | LGDFGIRLLCVG | 1.46 | P < 0.005 | 374 |
| 90 | 3545 | 631.86 | 65.15 | 2 | 1262.71 | LGDFGIRLLCVG | 1.42 | P < 0.005 | 374 |
| 92 | 1009 | 453.48 | 61.83 | 4 | 1810.90 | FDDQNLRSVNGAEITM | 1.75 | P < 0.005 | 375 |
| 92 | 1008 | 453.48 | 63.16 | 4 | 1810.90 | FDDQNLRSVNGAEITM | 1.63 | CountDiff | 375 |
| 93 | 5 | 300.89 | 36.97 | 4 | 1200.54 | ELDSQLNEPR | 1.31 | P < 0.005 | 376 |
| 93 | 557 | 400.85 | 36.97 | 3 | 1200.53 | ELDSQLNEPR | 1.28 | P < 0.005 | 376 |
| 94 | 3038 | 1045.53 | 49.91 | 1 | 1045.53 | KTTNQNVIK | 1.60 | P < 0.005 | 377 |
| 95 | 290 | 359.53 | 64.79 | 3 | 1076.57 | LSSWVLLMK | 1.17 | P < 0.05 | 378 |
| 95 | 1612 | 538.79 | 64.80 | 2 | 1076.57 | LSSWVLLMK | 1.16 | P < 0.05 | 378 |
| 96 | 175 | 339.91 | 33.97 | 4 | 1356.62 | TLVITSTPASPNR | 1.41 | P < 0.005 | 379 |
| 97 | 395 | 378.87 | 54.20 | 3 | 1134.59 | KGAAKVMVTNV | 1.42 | P < 0.01 | 380 |
| 97 | 1800 | 567.80 | 54.20 | 2 | 1134.59 | KGAAKVMVTNV | 1.41 | P < 0.05 | 380 |
| 98 | 2118 | 619.64 | 70.80 | 3 | 1856.90 | TEM#RNSENKNIFCVR | 1.57 | P < 0.05 | 381 |
| 100 | 2314 | 656.79 | 37.28 | 2 | 1312.57 | TQTVECTQTGSV | 1.77 | P < 0.005 | 382 |
| 101 | 2642 | 752.32 | 51.69 | 2 | 1503.63 | KMKEAAQRYQYA | 1.81 | P < 0.005 | 383 |
| 101 | 1358 | 501.88 | 51.70 | 3 | 1503.62 | KMKEAAQRYQYA | 1.69 | P < 0.05 | 383 |
| 101 | 1359 | 502.22 | 53.13 | 3 | 1504.64 | KMKEAAQRYQYA | 1.15 | CountDiff | 383 |
| 102 | 1134 | 470.92 | 48.06 | 3 | 1410.74 | PREEQFNSTFR | 1.51 | P < 0.05 | 384 |
| 104 | 2573 | 730.34 | 55.04 | 3 | 2189.00 | MGPGGGKAKALGGAGSGSKGSAGGGSK | 1.44 | P < 0.05 | 385 |
| 106 | 2557 | 724.33 | 60.23 | 3 | 2170.97 | TGNNRINITETGQLMVKDF | 1.38 | P < 0.05 | 386 |
| 107 | 3188 | 419.20 | 43.79 | 2 | 837.39 | LELFMGK | 1.34 | P < 0.05 | 387 |
| 108 | 1840 | 573.00 | 57.32 | 4 | 2288.98 | ELGVDQESEEGKGKTSPDKQK | 1.77 | P < 0.05 | 388 |
| 109 | 834 | 433.70 | 35.46 | 2 | 866.39 | NANAVCDT | 1.04 | P < 0.05 | 389 |
| 115 | 1392 | 506.78 | 66.28 | 2 | 1012.55 | MPQVFNFL | 1.62 | CountDiff | 390 |
| 116 | 2865 | 857.43 | 61.84 | 2 | 1713.85 | IAPQLSTEELVSLGEK | 1.26 | CountDiff | 391 |
| 117 | 2206 | 634.01 | 74.34 | 3 | 1900.01 | ECGKAFYSGSSLTQHQR | 1.10 | CountDiff | 392 |
| 118 | 2158 | 626.85 | 57.53 | 2 | 1252.69 | FVPQDVPPEPK | 1.07 | CountDiff | 393 |
| 119 | 4195 | 359.67 | 43.13 | 2 | 718.33 | LTLDEK | 1.32 | CountDiff | 394 |
| 120 | 4701 | 622.28 | 46.24 | 3 | 1864.82 | DIQMTQSPSSVSASVGDR | 5.18 | CountDiff | 395 |

TABLE 4
Identified Protein Fragments Decreased in Subjects having RA

| RA | | | | | | | | | SEQ |
|---|---|---|---|---|---|---|---|---|---|
| Marker | Fragment | | R.T. | | | | Fold | P | ID |
| # | # | m/z | (min.) | z | M + H | Peptide | Change | used | NO: |
| 1 | 2489 | 708.82 | 47.06 | 2 | 1416.63 | SVIPSDGPSVACVK | −1.11 | P < 0.05 | 38 |
| 1 | 774 | 428.52 | 47.75 | 3 | 1283.54 | EGYYGYTGAFR | −1.12 | P < 0.05 | 37 |
| 1 | 2324 | 659.78 | 32.05 | 2 | 1318.55 | WCAVSEHEATK | −1.12 | P < 0.05 | 36 |
| 1 | 1275 | 489.72 | 38.31 | 2 | 978.43 | DGAGDVAFVK | −1.12 | P < 0.005 | 35 |
| 1 | 1645 | 543.93 | 62.19 | 3 | 1629.77 | EDPQTFYYAVAVVK | −1.13 | P < 0.01 | 34 |
| 1 | 2251 | 642.26 | 47.74 | 2 | 1283.51 | EGYYGYTGAFR | −1.13 | P < 0.01 | 37 |
| 1 | 414 | 381.42 | 44.02 | 4 | 1522.66 | LKCDEWSVNSVGK | −1.15 | P < 0.05 | 33 |
| 1 | 1398 | 508.22 | 44.02 | 3 | 1522.64 | LKCDEWSVNSVGK | −1.16 | P < 0.001 | 33 |
| 1 | 58 | 315.18 | 16.87 | 2 | 629.35 | AVGNLR | −1.16 | P < 0.05 | 32 |
| 1 | 1306 | 493.57 | 62.53 | 3 | 1478.69 | MYLGYEYVTAIR | −1.16 | P < 0.05 | 31 |
| 1 | 2671 | 761.85 | 44.03 | 2 | 1522.69 | LKCDEWSVNSVGK | −1.17 | P < 0.05 | 33 |
| 1 | 1995 | 598.75 | 27.73 | 2 | 1196.49 | WCALSHHER | −1.17 | P < 0.01 | 30 |
| 1 | 928 | 444.69 | 10.13 | 2 | 888.37 | SCHTAVGR | −1.18 | P < 0.05 | 29 |
| 1 | 2732 | 789.38 | 72.33 | 2 | 1577.75 | TAGWNIPMGLLYNK | −1.18 | P < 0.05 | 28 |
| 1 | 1992 | 598.26 | 44.19 | 2 | 1195.51 | DSGFQMNQLR | −1.18 | P < 0.005 | 27 |
| 1 | 541 | 399.18 | 44.19 | 3 | 1195.52 | DSGFQMNQLR | −1.18 | P < 0.05 | 27 |
| 1 | 2598 | 739.85 | 62.52 | 2 | 1478.69 | MYLGYEYVTAIR | −1.18 | P < 0.05 | 31 |
| 1 | 3928 | 853.85 | 54.27 | 2 | 1706.69 | FDEFFSEGC*APGSKK | −1.18 | CountDiff | 26 |
| 1 | 1063 | 461.68 | 31.61 | 2 | 922.35 | DDTVCLAK | −1.19 | P < 0.001 | 25 |
| 1 | 1478 | 518.97 | 60.75 | 4 | 2072.86 | SDNCEDTPEAGYFAVAVVK | −1.20 | P < 0.05 | 24 |
| 1 | 1539 | 526.59 | 72.31 | 3 | 1577.75 | TAGWNIPMGLLYNK | −1.20 | P < 0.05 | 28 |
| 1 | 2425 | 691.62 | 60.77 | 3 | 2072.84 | SDNCEDTPEAGYFAVAVVK | −1.20 | P < 0.005 | 24 |
| 1 | 1811 | 569.57 | 51.09 | 3 | 1706.69 | FDEFFSEGC*APGSKK | −1.21 | P < 0.05 | 26 |
| 1 | 2292 | 651.96 | 58.15 | 3 | 1953.86 | NLNEKDYELLCLDGTR | −1.21 | P < 0.05 | 23 |
| 1 | 2674 | 762.34 | 46.07 | 2 | 1523.67 | LKCDEWSVNSVGK | −1.21 | P < 0.05 | 33 |
| 1 | 1423 | 511.88 | 58.92 | 3 | 1533.62 | CSTSSLLEACTFR | −1.22 | P < 0.005 | 22 |
| 1 | 653 | 414.19 | 30.59 | 2 | 827.37 | NPDPWAK | −1.22 | P < 0.005 | 21 |
| 1 | 729 | 423.45 | 39.76 | 4 | 1690.78 | DCHLAQVPSHTVVAR | −1.22 | P < 0.05 | 20 |
| 1 | 2686 | 767.30 | 58.92 | 2 | 1533.59 | CSTSSLLEACTFR | −1.22 | P < 0.005 | 22 |
| 1 | 1773 | 564.27 | 39.76 | 3 | 1690.79 | DCHLAQVPSHTVVAR | −1.24 | P < 0.01 | 20 |
| 1 | 2936 | 922.40 | 31.62 | 1 | 922.40 | DDTVCLAK | −1.24 | P < 0.05 | 25 |
| 1 | 2922 | 910.37 | 34.12 | 2 | 1819.73 | EGTCPEAPTDECKPVK | −1.25 | P < 0.05 | 19 |
| 1 | 2053 | 607.24 | 34.11 | 3 | 1819.70 | EGTCPEAPTDECKPVK | −1.25 | P < 0.001 | 19 |
| 1 | 905 | 442.21 | 37.46 | 3 | 1324.61 | KDSGFQMNQLR | −1.26 | P < 0.05 | 18 |
| 1 | 3271 | 489.23 | 58.13 | 4 | 1953.90 | NLNEKDYELLCLDGTR | −1.26 | P < 0.05 | 23 |
| 1 | 2247 | 641.26 | 43.62 | 2 | 1281.51 | CDEWSVNSVGK | −1.26 | P < 0.005 | 17 |
| 1 | 744 | 425.54 | 47.59 | 3 | 1274.60 | HSTIFENLANK | −1.27 | P < 0.005 | 16 |
| 1 | 4461 | 652.30 | 61.93 | 3 | 1954.88 | NLNEKDYELLCLDGTR | −1.27 | P < 0.05 | 23 |
| 1 | 1400 | 508.56 | 46.07 | 3 | 1523.66 | LKCDEWSVNSVGK | −1.28 | P < 0.005 | 33 |
| 1 | 3036 | 1036.94 | 60.74 | 2 | 2072.87 | SDNCEDTPEAGYFAVAVVK | −1.29 | P < 0.05 | 24 |
| 1 | 2378 | 678.29 | 60.45 | 2 | 1355.57 | DYELLCLDGTR | −1.29 | P < 0.005 | 15 |
| 1 | 4046 | 992.42 | 69.61 | 3 | 2975.24 | LCMGSGLNLCEPNNKEGYYGYTGAFR | −1.30 | P < 0.05 | 14 |
| 1 | 2806 | 827.39 | 30.57 | 1 | 827.39 | NPDPWAK | −1.32 | P < 0.05 | 21 |
| 1 | 997 | 452.53 | 60.45 | 3 | 1355.57 | DYELLCLDGTR | −1.32 | P < 0.001 | 15 |
| 1 | 2693 | 770.83 | 59.06 | 2 | 1540.65 | DQYELLCLDNTR | −1.33 | P < 0.001 | 13 |
| 1 | 1436 | 514.22 | 59.06 | 3 | 1540.64 | DQYELLCLDNTR | −1.37 | P < 0.001 | 13 |
| 1 | 2092 | 613.81 | 54.09 | 2 | 1226.61 | SLDGGFVYIAGK | −1.38 | P < 0.001 | 12 |
| 1 | 1812 | 569.57 | 48.82 | 3 | 1706.69 | FDEFFSEGC*APGSKK | −1.39 | P < 0.05 | 26 |
| 1 | 2166 | 628.27 | 54.36 | 3 | 1882.79 | ADRDQYELLCLDNTR | −1.40 | P < 0.001 | 11 |
| 1 | 3963 | 898.46 | 55.62 | 1 | 898.46 | SKEFQLF | −1.40 | P < 0.05 | 10 |
| 1 | 2962 | 941.91 | 54.32 | 2 | 1882.81 | ADRDQYELLCLDNTR | −1.43 | P < 0.005 | 11 |
| 1 | 1138 | 471.46 | 54.37 | 4 | 1882.82 | ADRDQYELLCLDNTR | −1.43 | P < 0.001 | 11 |
| 1 | 1868 | 576.25 | 43.29 | 2 | 1151.49 | LKCDEWSVN | −1.44 | P < 0.001 | 9 |
| 1 | 622 | 409.54 | 54.10 | 3 | 1226.60 | SLDGGFVYIAGK | −1.44 | P < 0.01 | 12 |
| 1 | 3353 | 530.24 | 35.82 | 3 | 1588.70 | KPVEEYANCHLAR | −1.45 | P < 0.01 | 8 |
| 1 | 3035 | 1035.50 | 90.27 | 2 | 2069.99 | EDLIWELLNQAQEHFGK | −1.50 | P < 0.05 | 7 |
| 1 | 745 | 425.55 | 43.73 | 3 | 1274.63 | HSTIFENLANK | −1.54 | P < 0.01 | 16 |
| 1 | 2295 | 652.34 | 83.60 | 2 | 1303.67 | SAGWNIPIGLLY | −1.58 | P < 0.005 | 6 |
| 1 | 1022 | 456.75 | 39.14 | 2 | 912.49 | YYAVAVVK | −1.63 | P < 0.005 | 5 |
| 1 | 1903 | 582.26 | 46.01 | 2 | 1163.51 | LYCDLPEPR | −1.64 | P < 0.001 | 4 |
| 1 | 2577 | 732.70 | 66.92 | 3 | 2196.08 | DAYLAPNNLKPVVAEFYGSK | −1.66 | P < 0.001 | 3 |
| 1 | 977 | 449.73 | 55.53 | 2 | 898.45 | SKEFQLF | −1.70 | P < 0.001 | 10 |
| 1 | 2103 | 616.33 | 58.44 | 3 | 1846.97 | LAPNNLKPVVAEFYGSK | −1.96 | P < 0.005 | 2 |
| 1 | 1871 | 576.57 | 46.47 | 3 | 1727.69 | IECVSAETTEDCIAK | −3.06 | P < 0.01 | 1 |
| 2 | 2144 | 625.28 | 50.38 | 2 | 1249.55 | LGMFNIQHCK | −1.15 | P < 0.05 | 39 |
| 2 | 678 | 417.20 | 50.38 | 3 | 1249.58 | LGMFNIQHCK | −1.18 | P < 0.005 | 39 |
| 3 | 4494 | 720.78 | 38.34 | 2 | 1440.55 | TEGDGVYTLNNEK | −1.17 | CountDiff | 41 |
| 3 | 1054 | 460.70 | 32.50 | 2 | 920.39 | GSFPWQAK | −1.18 | P < 0.05 | 40 |
| 4 | 3973 | 905.44 | 85.21 | 3 | 2714.30 | HYYIAAEEIIWNYAPSGIDIFTK | −1.01 | CountDiff | 43 |
| 4 | 4732 | 1191.63 | 70.02 | 1 | 1191.63 | DIFTGLIGPMK | −1.03 | CountDiff | 42 |
| 4 | 536 | 397.87 | 70.00 | 3 | 1191.59 | DIFTGLIGPMK | −1.13 | CountDiff | 42 |
| 5 | 2106 | 616.95 | 66.74 | 3 | 1848.83 | QFSFPLSSEPFQGSYK | −1.15 | P < 0.01 | 63 |
| 5 | 1722 | 558.27 | 62.48 | 3 | 1672.79 | TEHPFTVEEFVLPK | −1.21 | P < 0.05 | 62 |
| 5 | 2093 | 614.26 | 44.57 | 2 | 1227.51 | YDVENCLANK | −1.23 | P < 0.05 | 61 |
| 5 | 2679 | 765.34 | 20.66 | 2 | 1529.67 | TAQEGDHGSHVYTK | −1.24 | P < 0.05 | 60 |
| 5 | 1681 | 550.28 | 51.89 | 2 | 1099.55 | QTVSWAVTPK | −1.24 | P < 0.05 | 59 |
| 5 | 3176 | 409.86 | 41.19 | 3 | 1227.56 | YDVENCLANK | −1.25 | P < 0.05 | 61 |
| 5 | 581 | 403.70 | 22.11 | 2 | 806.39 | GPTQEFK | −1.25 | P < 0.05 | 58 |
| 5 | 1739 | 559.70 | 33.80 | 4 | 2235.78 | KYSDASDCHGEDSQAFCEK | −1.26 | P < 0.05 | 57 |
| 5 | 2470 | 703.24 | 36.04 | 3 | 2107.70 | YSDASDC*HGEDSQAFC*EK | −1.26 | P < 0.05 | 56 |
| 5 | 2848 | 848.77 | 63.56 | 3 | 2544.29 | SVSGKPQYMVLVPSLLHTETTEK | −1.26 | P < 0.05 | 55 |
| 5 | 2964 | 942.51 | 67.49 | 2 | 1884.01 | VSVQLEASPAFLAVPVEK | −1.26 | P < 0.05 | 54 |
| 5 | 2854 | 850.35 | 64.42 | 3 | 2549.03 | VYDYYETDEFAIAEYNAPCSK | −1.27 | P < 0.05 | 53 |
| 5 | 2623 | 745.94 | 33.80 | 3 | 2235.80 | KYSDASDCHGEDSQAFCEK | −1.27 | P < 0.05 | 57 |
| 5 | 1629 | 540.94 | 29.92 | 3 | 1620.80 | DNSVHWERPQKPK | −1.28 | P < 0.005 | 52 |
| 5 | 1687 | 552.29 | 41.86 | 2 | 1103.57 | SSGSLLNNAIK | −1.29 | P < 0.05 | 51 |
| 5 | 3099 | 1226.53 | 42.46 | 1 | 1226.53 | YDVENC*LANK | −1.29 | P < 0.05 | 50 |
| 5 | 2701 | 773.39 | 34.13 | 2 | 1545.77 | LVHVEEPHTETVR | −1.31 | P < 0.01 | 49 |
| 5 | 460 | 387.19 | 34.13 | 4 | 1545.74 | LVHVEEPHTETVR | −1.34 | CountDiff | 49 |
| 5 | 2358 | 671.85 | 47.89 | 2 | 1342.69 | AVLPTGDVIGDSAK | −1.36 | P < 0.05 | 48 |
| 5 | 2884 | 872.79 | 72.26 | 3 | 2616.35 | VLLAYLTAQPAPTSEDLTSATNIVK | −1.40 | P < 0.05 | 47 |
| 5 | 1452 | 515.93 | 34.11 | 3 | 1545.77 | LVHVEEPHTETVR | −1.46 | P < 0.005 | 49 |
| 5 | 1486 | 519.48 | 54.00 | 4 | 2074.90 | MCPQLQQYEMHGPEGLR | −1.47 | P < 0.05 | 46 |
| 5 | 2428 | 692.63 | 54.84 | 3 | 2075.87 | MCPQLQQYEMHGPEGLR | −1.63 | P < 0.001 | 46 |
| 5 | 3063 | 1103.60 | 41.87 | 1 | 1103.60 | SSGSLLNNAIK | −1.69 | P < 0.005 | 51 |
| 5 | 1435 | 513.79 | 43.84 | 2 | 1026.57 | TVLQDVPVR | −2.05 | P < 0.001 | 45 |
| 5 | 3447 | 587.33 | 55.43 | 2 | 1173.65 | FTVLQDVPVR | −2.08 | P < 0.05 | 44 |
| 6 | 1470 | 518.26 | 70.75 | 3 | 1552.76 | RHPYFYAPELLF | −1.02 | CountDiff | 85 |
| 6 | 3722 | 722.81 | 36.79 | 2 | 1444.61 | YICENQDSISSK | −1.05 | CountDiff | 84 |
| 6 | 3387 | 555.56 | 42.99 | 3 | 1664.66 | YKAAFTEC*C*QAADK | −1.09 | CountDiff | 83 |
| 6 | 1858 | 575.28 | 46.40 | 2 | 1149.55 | LVNEVTEFAK | −1.13 | P < 0.01 | 82 |
| 6 | 2357 | 671.79 | 66.50 | 2 | 1342.57 | AVMDDFAAFVEK | −1.15 | P < 0.05 | 81 |
| 6 | 160 | 337.18 | 29.16 | 2 | 673.35 | AWAVAR | −1.15 | P < 0.05 | 80 |
| 6 | 92 | 323.17 | 24.20 | 2 | 645.33 | LDELR | −1.15 | P < 0.05 | 79 |
| 6 | 2389 | 682.34 | 69.00 | 3 | 2045.00 | VFDEFKPLVEEPQNLIK | −1.18 | P < 0.005 | 78 |
| 6 | 1353 | 500.84 | 29.65 | 3 | 1500.50 | ADDKETCFAEEGK | −1.18 | P < 0.05 | 77 |
| 6 | 1664 | 547.29 | 49.10 | 3 | 1639.85 | KVPQVSTPTLVEVSR | −1.19 | P < 0.05 | 76 |
| 6 | 1823 | 570.72 | 36.08 | 2 | 1140.43 | CCTESLVNR | −1.19 | P < 0.05 | 75 |
| 6 | 1425 | 512.02 | 69.00 | 4 | 2045.06 | VFDEFKPLVEEPQNLIK | −1.19 | P < 0.05 | 78 |
| 6 | 1277 | 489.94 | 55.46 | 3 | 1467.80 | RHPDYSVVLLLR | −1.22 | P < 0.005 | 74 |
| 6 | 3618 | 663.80 | 67.08 | 4 | 2652.18 | LVRPEVDVMCTAFHDNEETFLK | −1.24 | CountDiff | 73 |
| 6 | 1866 | 575.79 | 44.55 | 2 | 1150.57 | LVNEVTEFAK | −1.25 | P < 0.05 | 82 |
| 6 | 1187 | 476.69 | 37.88 | 2 | 952.37 | DLGEENFK | −1.25 | P < 0.05 | 72 |
| 6 | 1031 | 458.51 | 37.01 | 3 | 1373.51 | AAFTECCQAADK | −1.27 | P < 0.05 | 71 |
| 6 | 3031 | 1023.03 | 69.00 | 2 | 2045.05 | VFDEFKPLVEEPQNLIK | −1.29 | P < 0.05 | 78 |
| 6 | 4029 | 967.00 | 63.89 | 2 | 1932.99 | SLHTLFGDKLCTVATLR | −1.31 | P < 0.05 | 70 |
| 6 | 2406 | 687.25 | 37.01 | 2 | 1373.49 | AAFTECCQAADK | −1.31 | P < 0.005 | 71 |
| 6 | 1377 | 504.61 | 52.37 | 3 | 1511.81 | VPQVSTPTLVEVSR | −1.33 | P < 0.01 | 69 |
| 6 | 4078 | 1045.39 | 43.22 | 2 | 2089.77 | VHTECCHGDLLECADDR | −1.36 | P < 0.05 | 68 |
| 6 | 1300 | 492.73 | 29.96 | 2 | 984.45 | TYETTLEK | −1.36 | P < 0.005 | 67 |
| 6 | 2980 | 956.44 | 58.81 | 2 | 1911.87 | RPCFSALEVDETYVPK | −1.36 | P < 0.05 | 66 |
| 6 | 1199 | 478.72 | 58.77 | 4 | 1911.86 | RPCFSALEVDETYVPK | −1.37 | P < 0.05 | 66 |
| 6 | 2998 | 984.48 | 29.93 | 1 | 984.48 | TYETTLEK | −1.41 | P < 0.05 | 67 |
| 6 | 2234 | 637.96 | 58.76 | 3 | 1911.86 | RPCFSALEVDETYVPK | −1.42 | P < 0.005 | 66 |
| 6 | 1514 | 523.19 | 43.22 | 4 | 2089.74 | VHTECCHGDLLECADDR | −1.48 | P < 0.001 | 68 |
| 6 | 2447 | 697.25 | 43.22 | 3 | 2089.73 | VHTECCHGDLLECADDR | −1.48 | P < 0.001 | 69 |
| 6 | 969 | 449.24 | 78.58 | 3 | 1345.70 | FYAPELLFFAK | −1.64 | P < 0.05 | 65 |
| 6 | 1115 | 469.24 | 60.31 | 3 | 1405.70 | RHPYFYAPELL | −1.66 | P < 0.001 | 64 |
| 8 | 2027 | 602.28 | 57.48 | 3 | 1804.82 | AEDHFSVIDFNQNIR | −1.20 | P < 0.05 | 96 |
| 8 | 528 | 396.71 | 24.94 | 2 | 792.41 | ALYAQAR | −1.25 | P < 0.05 | 95 |
| 8 | 1922 | 585.26 | 47.58 | 2 | 1169.51 | SSALDMENFR | −1.27 | P < 0.05 | 94 |
| 8 | 2486 | 708.03 | 62.77 | 3 | 2122.07 | VVNNSPQPQNVVFDVQIPK | −1.27 | P < 0.05 | 93 |
| 8 | 732 | 423.74 | 32.33 | 2 | 846.47 | IYLQPGR | −1.29 | P < 0.01 | 92 |
| 8 | 2745 | 791.92 | 49.76 | 2 | 1582.83 | IQPSGGTNINEALLR | −1.31 | P < 0.05 | 91 |
| 8 | 338 | 367.94 | 31.40 | 4 | 1468.74 | AHVSFKPTVAQQR | −1.32 | P < 0.05 | 90 |
| 8 | 945 | 446.24 | 22.46 | 2 | 891.47 | VQSTITSR | −1.35 | P < 0.005 | 89 |
| 8 | 1551 | 528.28 | 49.74 | 3 | 1582.82 | IQPSGGTNINEALLR | −1.35 | P < 0.005 | 91 |
| 8 | 649 | 413.53 | 23.04 | 3 | 1238.57 | LSNENHGIAQR | −1.36 | P < 0.01 | 88 |
| 8 | 1278 | 490.26 | 31.39 | 3 | 1468.76 | AHVSFKPTVAQQR | −1.37 | P < 0.01 | 90 |
| 8 | 728 | 423.23 | 50.80 | 2 | 845.45 | TILDDLR | −1.43 | P < 0.001 | 87 |
| 8 | 1440 | 514.28 | 37.68 | 2 | 1027.55 | TEVNVLPGAK | −1.46 | P < 0.005 | 86 |
| 10 | 1896 | 580.80 | 61.15 | 2 | 1160.59 | WFYIASAFR | −1.23 | P < 0.05 | 97 |
| 11 | 2225 | 636.82 | 63.64 | 4 | 2544.26 | GFYPSDIAVEWESNGQPENNYK | −1.38 | P < 0.005 | 98 |
| 11 | 2227 | 636.99 | 63.94 | 4 | 2544.94 | GFYPSDIAVEWESNGQPENNYK | −16.67 | P < 0.05 | 98 |
| 12 | 2603 | 740.33 | 48.76 | 2 | 1479.65 | GTFATLSELHCDK | −1.14 | CountDiff | 105 |
| 12 | 1050 | 460.23 | 45.99 | 3 | 1378.67 | EFTPPVQAAYQK | −1.18 | P < 0.05 | 104 |
| 12 | 3032 | 1029.96 | 68.32 | 2 | 2058.91 | FFESFGDLSTPDAVMGNPK | −1.39 | P < 0.05 | 103 |
| 12 | 2270 | 647.55 | 62.52 | 4 | 2587.18 | GTFATLSELHC*DKLHVDPENFR | −1.51 | P < 0.05 | 102 |
| 12 | 2419 | 689.84 | 43.49 | 2 | 1378.67 | EFTPPVQAAYQK | −1.52 | P < 0.01 | 104 |
| 12 | 2405 | 686.97 | 68.34 | 3 | 2058.89 | FFESFGDLSTPDAVMGNPK | −1.60 | P < 0.005 | 103 |
| 12 | 878 | 438.88 | 46.08 | 3 | 1314.62 | VNVDEVGGEALGR | −1.71 | P < 0.05 | 101 |
| 12 | 2233 | 637.85 | 62.18 | 2 | 1274.69 | LLVVYPWTQR | −1.76 | P < 0.005 | 100 |
| 12 | 438 | 383.88 | 36.67 | 3 | 1149.62 | VVAGVANALAHK | −1.77 | P < 0.05 | 99 |
| 12 | 2319 | 657.82 | 46.12 | 2 | 1314.63 | VNVDEVGGEALGR | −1.92 | P < 0.001 | 101 |
| 12 | 1308 | 493.89 | 48.78 | 3 | 1479.65 | GTFATLSELHCDK | −2.12 | P < 0.005 | 105 |
| 13 | 866 | 437.71 | 27.44 | 2 | 874.41 | QLANGVDR | −1.15 | P < 0.05 | 107 |
| 13 | 863 | 437.24 | 28.51 | 2 | 873.47 | QLANGVDR | −1.23 | P < 0.05 | 107 |
| 13 | 325 | 365.70 | 26.66 | 2 | 730.39 | TLDPER | −1.33 | P < 0.05 | 106 |
| 15 | 3507 | 616.96 | 75.84 | 3 | 1848.86 | EQLQDMGLVDLFSPEK | −1.24 | P < 0.05 | 112 |
| 15 | 799 | 430.74 | 46.11 | 2 | 860.47 | LQPLDFK | −1.25 | P < 0.05 | 111 |
| 15 | 3595 | 655.30 | 46.04 | 2 | 1309.59 | DDLYVSDAFHK | −1.26 | P < 0.05 | 110 |
| 15 | 1410 | 510.24 | 50.55 | 3 | 1528.70 | FATTFYQHLADSK | −1.27 | P < 0.05 | 109 |
| 15 | 858 | 437.20 | 46.05 | 3 | 1309.58 | DDLYVSDAFHK | −1.43 | P < 0.01 | 110 |
| 15 | 3700 | 705.81 | 62.07 | 2 | 1410.61 | DIPMNPMC*IYR | −1.47 | P < 0.005 | 108 |
| 16 | 1933 | 587.28 | 43.64 | 5 | 2932.37 | FVSEAGPTGTGESKCPLMVKVLDAVRGSP | −1.08 | CountDiff | 117 |
| 16 | 3502 | 614.53 | 67.64 | 4 | 2455.10 | TSESGELHGLTTEEEFVEGIYK | −1.16 | P < 0.05 | 116 |
| 16 | 1019 | 456.24 | 53.99 | 3 | 1366.70 | GSPAINVAVHVFR | −1.26 | P < 0.05 | 115 |
| 16 | 4169 | 1394.68 | 47.33 | 1 | 1394.68 | AADDTWEPFASGK | −1,32 | P < 0.05 | 114 |
| 16 | 2397 | 683.87 | 54.00 | 2 | 1366.73 | GSPAINVAVHVFR | −1.33 | P < 0.01 | 115 |
| 16 | 2450 | 697.80 | 50.88 | 2 | 1394.59 | AADDTWEPFASGK | −1.35 | P < 0.01 | 114 |
| 16 | 3499 | 613.80 | 58.28 | 4 | 2452.18 | ALGISPFHEHAEVVFTANDSGPR | −1.38 | P < 0.01 | 113 |
| 17 | 1203 | 479.71 | 24.31 | 2 | 958.41 | HADPDFTR | −1.15 | P < 0.05 | 121 |
| 17 | 1311 | 494.58 | 28.67 | 3 | 1481.72 | IYGNQDTSSQLKK | −1.24 | P < 0.05 | 120 |
| 17 | 3849 | 797.33 | 53.74 | 3 | 2389.97 | MLADAPPQDPSC*C*SGALYYGSK | −1.25 | P < 0.05 | 119 |
| 17 | 2375 | 677.33 | 31.71 | 2 | 1353.65 | IYGNQDTSSQLK | −1.45 | P < 0.005 | 118 |
| 18 | 4799 | 735.95 | 57.96 | 3 | 2205.83 | MHSMNGFMYGNQPGLTMC*K | −1.09 | CountDiff | 122 |
| 19 | 4464 | 653.27 | 39.62 | 2 | 1305.53 | GQYC*YELDEK | −1.26 | CountDiff | 124 |
| 19 | 1170 | 474.88 | 46.42 | 3 | 1422.62 | FEDGVLDPDYPR | −1.41 | P < 0.05 | 123 |
| 20 | 261 | 354.20 | 29.67 | 2 | 707.39 | ANLFNK | −1.40 | P < 0.05 | 125 |
| 20 | 2483 | 707.40 | 29.66 | 1 | 707.40 | ANLFNK | −1.42 | P < 0.05 | 125 |
| 24 | 1698 | 554.29 | 61.79 | 4 | 2214.14 | DKLAAC*LEGNC*AEGLGTNYR | −1.16 | P < 0.05 | 127 |
| 24 | 1255 | 486.74 | 25.94 | 2 | 972.47 | YTACETAR | −1.44 | P < 0.05 | 126 |
| 25 | 2634 | 749.85 | 76.06 | 4 | 2996.38 | VADALTNAVAHVDDMPNALSALSDLHAHK | −1.38 | P < 0.05 | 130 |
| 25 | 2148 | 625.71 | 74.52 | 5 | 3124.52 | KVADALTNAVAHVDDMPNALSALSDLHAHK | −1.48 | P < 0.05 | 129 |
| 25 | 2012 | 600.08 | 76.07 | 5 | 2996.37 | VADALTNAVAHVDDMPNALSALSDLHAHK | −1.66 | P < 0.05 | 130 |
| 25 | 1414 | 510.57 | 37.39 | 3 | 1529.69 | VGAHAGEYGAEALER | −2.03 | P < 0.01 | 128 |
| 26 | 2102 | 615.85 | 71.57 | 2 | 1230.69 | QGLLPVLESFK | −1.29 | P < 0.05 | 132 |
| 26 | 1602 | 538.25 | 64.78 | 3 | 1612.73 | LLDNWDSVTSTFSK | −1.48 | P < 0.05 | 131 |
| 27 | 4121 | 1159.05 | 74.07 | 2 | 2317.09 | QSNNKYAASSYLSLTPEQWK | −1.09 | CountDiff | 133 |
| 28 | 3681 | 695.36 | 58.09 | 3 | 2084.06 | LQQVLHAGSGPCLPHLLSR | −1.15 | P < 0.05 | 134 |
| 30 | 1758 | 561.77 | 46.26 | 2 | 1122.53 | QVEGMEDWK | −1.54 | P < 0.005 | 135 |
| 32 | 378 | 375.22 | 32.72 | 2 | 749.43 | MLSLGTK | −1.66 | P < 0.001 | 137 |
| 33 | 321 | 365.20 | 21.50 | 2 | 729.39 | ATGIPDR | −1.87 | P < 0.05 | 138 |
| 34 | 3454 | 589.55 | 33.80 | 3 | 1766.63 | EEEQQRCESLAEVNT | −1.68 | P < 0.05 | 139 |
| 34 | 4248 | 442.41 | 33.78 | 4 | 1766.62 | EEEQQRCESLAEVNT | −1.80 | P < 0.05 | 139 |
| 35 | 2568 | 727.88 | 67.66 | 2 | 1454.75 | MNQLTQELFSLK | −1.55 | P < 0.01 | 140 |
| 38 | 4762 | 693.90 | 62.09 | 2 | 1386.79 | NVPLPVIAELPPK | −1.22 | CountDiff | 142 |
| 38 | 807 | 431.73 | 29.49 | 2 | 862.45 | VTSTLTIK | −3.58 | CountDiff | 141 |
| 40 | 2977 | 953.43 | 68.34 | 2 | 1905.85 | TTPPMLDSDGSFFLYSK | −1.38 | P < 0.05 | 144 |
| 40 | 2001 | 598.98 | 69.85 | 3 | 1794.92 | VVSVLTVVHQDWLNGK | −1.46 | CountDiff | 143 |
| 40 | 2221 | 635.99 | 67.72 | 3 | 1905.95 | TTPPMLDSDGSFFLYSK | −1.64 | P < 0.05 | 144 |
| 42 | 1908 | 582.74 | 39.88 | 2 | 1164.47 | LPECEAVCGK | −5.08 | CountDiff | 145 |
| 51 | 1431 | 513.00 | 52.30 | 4 | 2048.98 | RLYGSEAFATDFQDSAAAK | −1.15 | P < 0.05 | 146 |
| 52 | 1702 | 554.57 | 53.77 | 3 | 1661.69 | EHAVEGDCDFQLLK | −1.28 | P < 0.005 | 147 |
| 53 | 2625 | 746.29 | 48.62 | 3 | 2236.85 | KEDSC*QLGYSAGPC*MGMTSR | −1.33 | P < 0.05 | 148 |
| 54 | 1137 | 471.28 | 43.44 | 2 | 941.55 | EQLTPLIK | −1.44 | P < 0.05 | 149 |
| 56 | 553 | 400.50 | 25.61 | 3 | 1199.48 | EQHPDMSVTR | −1.26 | P < 0.05 | 150 |
| 57 | 3610 | 660.32 | 50.55 | 2 | 1319.63 | AGALNSNDAFVLK | −7.09 | P < 0.01 | 151 |
| 59 | 2343 | 668.66 | 68.53 | 3 | 2003.96 | GSLVQASEANLQAAQDFVR | −1.19 | P < 0.05 | 152 |
| 60 | 1585 | 534.20 | 53.42 | 3 | 1600.58 | IASFSQNC*DIYPGK | −1.21 | P < 0.05 | 153 |
| 61 | 2933 | 921.35 | 60.09 | 4 | 3682.38 | CGLVPVLAENYKSQQSSDPDPNCVDRPVEGYLA | −1.28 | P < 0.05 | 154 |
| 62 | 829 | 433.23 | 40.61 | 3 | 1297.67 | SLEDLQLTHNK | −1.02 | CountDiff | 156 |
| 62 | 2088 | 613.30 | 50.92 | 2 | 1225.59 | ISNIPDEYFK | −1.55 | P < 0.001 | 155 |
| 66 | 931 | 445.21 | 60.18 | 2 | 889.41 | QNGGLATVE | −1.54 | P < 0.005 | 157 |
| 70 | 1614 | 539.25 | 72.38 | 3 | 1615.73 | M#C*EQALGKGC*GGDSK | −1.31 | P < 0.005 | 158 |
| 71 | 3662 | 689.69 | 69.01 | 3 | 2067.05 | LLNLDGTC*ADSYSFVFSR | −1.34 | P < 0.001 | 159 |
| 72 | 1729 | 558.79 | 42.03 | 2 | 1116.57 | AGKSTFLKKH | −1.21 | P < 0.01 | 160 |
| 72 | 3069 | 1116.60 | 42.02 | 1 | 1116.60 | AGKSTFLKKH | −1.32 | P < 0.05 | 160 |
| 74 | 4442 | 634.61 | 49.49 | 3 | 1901.81 | EIVMTQSPATLSVSPGER | −1.26 | P < 0.05 | 161 |
| 75 | 3442 | 586.29 | 46.40 | 2 | 1171.57 | EGLCCGPSIPPV | −1.20 | P < 0.05 | 162 |
| 79 | 1350 | 500.72 | 42.14 | 2 | 1000.43 | AAYMNKER | −1.18 | P < 0.01 | 163 |
| 79 | 3015 | 1000.48 | 42.14 | 1 | 1000.48 | AAYMNKER | −1.22 | P < 0.05 | 163 |
| 81 | 1342 | 499.55 | 58.16 | 3 | 1496.63 | YYCFQGNQFLR | −1.23 | P < 0.05 | 164 |
| 82 | 1103 | 467.88 | 38.36 | 3 | 1401.62 | GGCLPPC*DGGPKSR | −1.35 | P < 0.05 | 165 |
| 86 | 4438 | 632.30 | 61.38 | 2 | 1263.59 | ASDDDVGENARI | −1.17 | P < 0.05 | 166 |
| 88 | 4067 | 1019.60 | 54.28 | 1 | 1019.60 | EEAIAVTMR | −1.27 | P < 0.05 | 167 |
| 91 | 1495 | 520.91 | 39.58 | 3 | 1560.71 | YNPDSGLEVLAVQR | −1.45 | P < 0.05 | 168 |
| 95 | 4751 | 400.56 | 53.85 | 3 | 1199.66 | IVDLVKELDR | −1.29 | CountDiff | 169 |
| 99 | 3280 | 494.73 | 36.75 | 4 | 1975.90 | HKLIHTGVKSHACEQCGK | −1.19 | P < 0.05 | 170 |
| 103 | 3639 | 671.30 | 61.56 | 3 | 2011.88 | VFWRSSGLPHPSQAQSAR | −1.20 | P < 0.05 | 171 |
| 105 | 3190 | 420.21 | 49.15 | 4 | 1677.82 | GNALSVC*SRESPGSKK | −1.22 | P < 0.05 | 172 |
| 110 | 3018 | 1000.93 | 62.13 | 4 | 4000.70 | CLQRIVTKLQMEAGLCEEQLNQADALLQSDVRL- | −1.22 | CountDiff | 173 |
| | | | | | | LAA | | | |
| 110 | 4159 | 1334.21 | 62.14 | 3 | 4000.61 | CLQRIVTKLQMEAGLCEEQLNQADALLQSDVRL- | −1.44 | CountDiff | 173 |
| | | | | | | LAA | | | |
| 111 | 2673 | 762.06 | 67.89 | 3 | 2284.16 | IITHPNFNGNTLDNDIMLIK | −1.09 | CountDiff | 174 |
| 112 | 1269 | 489.00 | 72.92 | 4 | 1952.98 | FTVDRPFLFLIYEHR | −1.06 | CountDiff | 175 |
| 113 | 4790 | 502.25 | 42.95 | 2 | 1003.49 | GGSIFGLAPGK | −1.28 | CountDiff | 176 |
| 114 | 1429 | 512.78 | 45.54 | 2 | 1024.55 | GQGKPPVWR | −1.17 | CountDiff | 111 |
| 3^{†} | 4777 | 1145.50 | 50.05 | 3 | 3434.48 | AVGDKLPECEADDGCPKPPEIAHGYVEHSVR | NA | CountDiff | 178 |

^{†}Trend was neutral rather than decreased

TABLE 5
Unidentified Proteins Increased in Subjects having RA

| Component | | R.T. | | | Fold | |
|---|---|---|---|---|---|---|
| # | m/z | (min.) | z | M + H | Change | P |
| 1929 | 585.94 | 56.22 | 3 | 1755.8 | 1.01 | CountDiff |
| 1129 | 470.7 | 39.77 | 2 | 940.39 | 1.02 | CountDiff |
| 3401 | 561.79 | 52.59 | 2 | 1122.57 | 1.02 | CountDiff |
| 423 | 382.54 | 35.58 | 3 | 1145.6 | 1.02 | CountDiff |
| 4779 | 362.87 | 40.15 | 3 | 1086.59 | 1.03 | CountDiff |
| 171 | 339.71 | 12.84 | 2 | 678.41 | 1.04 | CountDiff |
| 150 | 335.68 | 25.8 | 1 | 335.68 | 1.04 | CountDiff |
| 225 | 349.18 | 29.85 | 2 | 697.35 | 1.04 | CountDiff |
| 3864 | 805.48 | 51.64 | 1 | 805.48 | 1.05 | CountDiff |
| 2403 | 686.64 | 54.04 | 3 | 2057.9 | 1.05 | CountDiff |
| 4200 | 371.46 | 14.54 | 3 | 1112.36 | 1.06 | CountDiff |
| 3982 | 910.07 | 26.24 | 4 | 3637.26 | 1.06 | CountDiff |
| 54 | 314.22 | 42.28 | 2 | 627.43 | 1.07 | P < 0.05 |
| 4667 | 633.32 | 61.28 | 1 | 633.32 | 1.07 | CountDiff |
| 1303 | 493.2 | 35.92 | 3 | 1477.58 | 1.09 | CountDiff |
| 4597 | 625.32 | 22.97 | 1 | 625.32 | 1.09 | CountDiff |
| 3954 | 883.38 | 73.84 | 2 | 1765.75 | 1.09 | CountDiff |
| 547 | 399.85 | 24.21 | 3 | 1197.53 | 1.09 | CountDiff |
| 1428 | 512.6 | 66.52 | 3 | 1535.78 | 1.1 | CountDiff |
| 4691 | 1152.25 | 84.26 | 4 | 4605.98 | 1.1 | CountDiff |
| 4104 | 1089.98 | 96.74 | 5 | 5445.87 | 1.1 | CountDiff |
| 4211 | 398.2 | 37.52 | 2 | 795.39 | 1.11 | P < 0.05 |
| 4087 | 1062.43 | 48.57 | 1 | 1062.43 | 1.12 | CountDiff |
| 1457 | 516.26 | 23.89 | 1 | 516.26 | 1.12 | CountDiff |
| 3344 | 527.27 | 54.34 | 2 | 1053.53 | 1.13 | P < 0.05 |
| 1055 | 460.71 | 44.94 | 2 | 920.41 | 1.15 | P < 0.05 |
| 3659 | 686.97 | 53.57 | 3 | 2058.89 | 1.15 | CountDiff |
| 1754 | 561.26 | 80.75 | 3 | 1681.76 | 1.16 | P < 0.05 |
| 219 | 347.71 | 31.81 | 2 | 694.41 | 1.17 | P < 0.05 |
| 1235 | 483.74 | 34.29 | 2 | 966.47 | 1.17 | CountDiff |
| 974 | 449.54 | 51.44 | 3 | 1346.6 | 1.17 | CountDiff |
| 1227 | 482.26 | 39.67 | 2 | 963.51 | 1.17 | CountDiff |
| 4776 | 1093.82 | 57.68 | 3 | 3279.44 | 1.17 | NA |
| 1627 | 540.81 | 64.79 | 1 | 540.81 | 1.18 | P < 0.005 |
| 4697 | 565.26 | 54.05 | 1 | 565.26 | 1.18 | CountDiff |
| 165 | 339.2 | 54.33 | 3 | 1015.58 | 1.19 | P < 0.005 |
| 1939 | 588.91 | 52.16 | 3 | 1764.71 | 1.19 | P < 0.05 |
| 2408 | 687.43 | 31.01 | 1 | 687.43 | 1.19 | P < 0.05 |
| 1549 | 527.8 | 54.48 | 2 | 1054.59 | 1.19 | P < 0.05 |
| 4614 | 717.34 | 58.75 | 2 | 1433.67 | 1.19 | CountDiff |
| 788 | 429.73 | 35.02 | 2 | 858.45 | 1.2 | P < 0.05 |
| 717 | 422.22 | 23.02 | 2 | 843.43 | 1.2 | P < 0.05 |
| 3354 | 530.3 | 54.32 | 2 | 1059.59 | 1.2 | P < 0.05 |
| 4083 | 1057.41 | 49.26 | 4 | 4226.62 | 1.2 | P < 0.05 |
| 3157 | 382.22 | 33.47 | 2 | 763.43 | 1.21 | P < 0.05 |
| 205 | 344.91 | 40.1 | 5 | 1720.52 | 1.22 | P < 0.01 |
| 36 | 309.68 | 26.8 | 2 | 618.35 | 1.22 | P < 0.05 |
| 1111 | 468.74 | 33.42 | 2 | 936.47 | 1.22 | P < 0.05 |
| 2658 | 755.34 | 80.21 | 2 | 1509.67 | 1.22 | CountDiff |
| 4634 | 984.81 | 84.96 | 5 | 4920.02 | 1.22 | CountDiff |
| 3413 | 566.78 | 50.07 | 2 | 1132.55 | 1.23 | P < 0.005 |
| 1313 | 495.21 | 57.34 | 3 | 1483.61 | 1.23 | P < 0.05 |
| 131 | 332.15 | 29.8 | 2 | 663.29 | 1.23 | P < 0.05 |
| 3173 | 404.7 | 39.1 | 2 | 808.39 | 1.23 | CountDiff |
| 880 | 438.97 | 56.3 | 4 | 1752.86 | 1.24 | P < 0.01 |
| 3462 | 594.57 | 73.89 | 3 | 1781.69 | 1.24 | P < 0.05 |
| 890 | 440.2 | 47.63 | 2 | 879.39 | 1.24 | P < 0.05 |
| 164 | 338.7 | 11.4 | 2 | 676.39 | 1.24 | P < 0.05 |
| 4770 | 832.47 | 54.81 | 1 | 832.47 | 1.24 | NA |
| 1492 | 520.34 | 29.2 | 1 | 520.34 | 1.25 | P < 0.05 |
| 850 | 436.19 | 50.2 | 3 | 1306.55 | 1.25 | P < 0.05 |
| 1801 | 567.8 | 71.05 | 2 | 1134.59 | 1.25 | P < 0.05 |
| 2916 | 902.47 | 50.72 | 2 | 1803.93 | 1.26 | P < 0.05 |
| 1568 | 531.75 | 35.37 | 2 | 1062.49 | 1.26 | P < 0.05 |
| 2124 | 621.29 | 42.93 | 2 | 1241.57 | 1.27 | P < 0.05 |
| 3757 | 741.59 | 56.7 | 4 | 2963.34 | 1.27 | CountDiff |
| 203 | 344.68 | 25.84 | 2 | 688.35 | 1.28 | P < 0.005 |
| 3464 | 596.29 | 27.57 | 2 | 1191.57 | 1.28 | P < 0.005 |
| 3024 | 1015.6 | 54.32 | 1 | 1015.6 | 1.28 | P < 0.01 |
| 1943 | 589.25 | 73.91 | 3 | 1765.73 | 1.28 | P < 0.01 |
| 363 | 372.69 | 29.26 | 2 | 744.37 | 1.28 | P < 0.05 |
| 2759 | 800.73 | 62.02 | 5 | 3999.62 | 1.28 | P < 0.05 |
| 2067 | 609.32 | 30.2 | 1 | 609.32 | 1.28 | P < 0.05 |
| 2402 | 686.43 | 34.29 | 1 | 686.43 | 1.29 | P < 0.001 |
| 1920 | 584.97 | 56.3 | 3 | 1752.89 | 1.29 | P < 0.005 |
| 275 | 357.19 | 33.78 | 3 | 1069.55 | 1.29 | P < 0.005 |
| 2373 | 676.83 | 64.5 | 2 | 1352.65 | 1.29 | P < 0.05 |
| 1228 | 482.27 | 53.95 | 2 | 963.53 | 1.31 | P < 0.01 |
| 2203 | 633.77 | 43.97 | 4 | 2532.06 | 1.31 | P < 0.01 |
| 3135 | 328.22 | 10.99 | 2 | 655.43 | 1.31 | P < 0.05 |
| 1966 | 593.8 | 48.43 | 2 | 1186.59 | 1.31 | P < 0.05 |
| 385 | 376.84 | 22.69 | 3 | 1128.5 | 1.31 | P < 0.05 |
| 3495 | 613.31 | 70.94 | 2 | 1225.61 | 1.32 | P < 0.005 |
| 1263 | 488.23 | 33.94 | 3 | 1462.67 | 1.32 | P < 0.01 |
| 505 | 393.21 | 18.66 | 2 | 785.41 | 1.32 | P < 0.01 |
| 831 | 433.23 | 37.51 | 2 | 865.45 | 1.32 | P < 0.05 |
| 1590 | 535.28 | 12.37 | 1 | 535.28 | 1.32 | P < 0.05 |
| 810 | 431.81 | 42.24 | 4 | 1724.22 | 1.32 | P < 0.05 |
| 2431 | 693.85 | 47.78 | 2 | 1386.69 | 1.33 | P < 0.01 |
| 2439 | 695.31 | 37.12 | 2 | 1389.61 | 1.33 | P < 0.01 |
| 4649 | 485.24 | 50.32 | 2 | 969.47 | 1.33 | P < 0.05 |
| 4569 | 411.2 | 38.23 | 1 | 411.2 | 1.33 | P < 0.05 |
| 1725 | 558.5 | 53.76 | 4 | 2230.98 | 1.33 | P < 0.05 |
| 4702 | 622.99 | 78.35 | 3 | 1866.95 | 1.33 | CountDiff |
| 984 | 450.72 | 37.19 | 2 | 900.43 | 1.34 | P < 0.05 |
| 4469 | 671.36 | 35.72 | 1 | 671.36 | 1.34 | P < 0.05 |
| 4679 | 758.83 | 55.08 | 4 | 3032.3 | 1.35 | P < 0.05 |
| 4654 | 525.73 | 54.66 | 5 | 2624.62 | 1.35 | CountDiff |
| 3 | 300.17 | 12.05 | 2 | 599.33 | 1.36 | P < 0.01 |
| 3607 | 659.82 | 64.34 | 4 | 2636.26 | 1.36 | P < 0.05 |
| 1162 | 473.75 | 41.42 | 2 | 946.49 | 1.36 | P < 0.05 |
| 2721 | 785.44 | 18.65 | 1 | 785.44 | 1.37 | P < 0.01 |
| 4600 | 633.8 | 60.37 | 1 | 633.8 | 1.37 | P < 0.05 |
| 1705 | 555.8 | 54.27 | 2 | 1110.59 | 1.38 | P < 0.001 |
| 330 | 366.2 | 27.4 | 2 | 731.39 | 1.38 | P < 0.005 |
| 118 | 329.17 | 31.91 | 2 | 657.33 | 1.38 | P < 0.05 |
| 1946 | 589.59 | 52.25 | 3 | 1766.75 | 1.38 | P < 0.05 |
| 3394 | 557.26 | 71.31 | 3 | 1669.76 | 1.38 | CountDiff |
| 3371 | 542.24 | 60.8 | 3 | 1624.7 | 1.39 | P < 0.001 |
| 433 | 383.52 | 44.97 | 3 | 1148.54 | 1.39 | P < 0.005 |
| 4032 | 973.52 | 45.19 | 1 | 973.52 | 1.39 | P < 0.05 |
| 720 | 422.23 | 25.64 | 2 | 843.45 | 1.39 | P < 0.05 |
| 1569 | 532 | 53.75 | 4 | 2124.98 | 1.39 | P < 0.05 |
| 4633 | 978.46 | 50.37 | 1 | 978.46 | 1.39 | CountDiff |
| 2723 | 785.92 | 66.44 | 2 | 1570.83 | 1.4 | P < 0.005 |
| 88 | 322.18 | 29.89 | 3 | 964.52 | 1.4 | P < 0.01 |
| 4237 | 426.21 | 35.62 | 1 | 426.21 | 1.41 | P < 0.001 |
| 2457 | 699.34 | 62.69 | 1 | 699.34 | 1.41 | P < 0.05 |
| 1733 | 559.4 | 53.2 | 5 | 2792.97 | 1.41 | P < 0.05 |
| 1704 | 555.78 | 50.1 | 2 | 1110.55 | 1.42 | P < 0.001 |
| 48 | 312.83 | 33.43 | 3 | 936.47 | 1.42 | P < 0.005 |
| 4323 | 511.28 | 22.86 | 2 | 1021.55 | 1.42 | P < 0.05 |
| 1565 | 531.28 | 52.2 | 2 | 1061.55 | 1.43 | P < 0.005 |
| 400 | 379.72 | 27.31 | 2 | 758.43 | 1.43 | P < 0.005 |
| 1727 | 558.73 | 21.44 | 2 | 1116.45 | 1.43 | P < 0.005 |
| 2368 | 674.85 | 64.55 | 2 | 1348.69 | 1.43 | P < 0.005 |
| 3342 | 526.22 | 28.38 | 3 | 1576.64 | 1.43 | P < 0.05 |
| 3541 | 630.96 | 62.7 | 3 | 1890.86 | 1.43 | P < 0.05 |
| 4757 | 567.25 | 92.73 | 4 | 2265.98 | 1.43 | CountDiff |
| 4007 | 940.16 | 74.57 | 4 | 3757.62 | 1.44 | P < 0.05 |
| 1571 | 532.26 | 48.01 | 3 | 1594.76 | 1.45 | P < 0.01 |
| 828 | 433.23 | 40.32 | 2 | 865.45 | 1.45 | P < 0.05 |
| 2136 | 624.32 | 51.59 | 3 | 1870.94 | 1.45 | P < 0.05 |
| 2982 | 960.09 | 35.93 | 3 | 2878.25 | 1.45 | CountDiff |
| 4659 | 563.26 | 38.51 | 2 | 1125.51 | 1.45 | CountDiff |
| 1694 | 553.69 | 50.84 | 2 | 1106.37 | 1.45 | CountDiff |
| 4719 | 858.75 | 71.63 | 1 | 858.75 | 1.45 | NA |
| 695 | 419.2 | 27.39 | 2 | 837.39 | 1.46 | P < 0.005 |
| 3518 | 623.32 | 51.82 | 3 | 1867.94 | 1.46 | P < 0.05 |
| 132 | 332.15 | 16.33 | 2 | 663.29 | 1.46 | P < 0.05 |
| 4642 | 1117.49 | 82.35 | 4 | 4466.94 | 1.46 | P < 0.05 |
| 2949 | 932.52 | 48.7 | 1 | 932.52 | 1.46 | CountDiff |
| 327 | 366.16 | 39.18 | 3 | 1096.46 | 1.47 | P < 0.01 |
| 1260 | 487.28 | 54.08 | 3 | 1459.82 | 1.47 | P < 0.05 |
| 2130 | 622.82 | 53.25 | 2 | 1244.63 | 1.47 | P < 0.05 |
| 4378 | 572.84 | 61.44 | 5 | 2860.17 | 1.47 | P < 0.05 |
| 2886 | 876.96 | 56.3 | 2 | 1752.91 | 1.48 | P < 0.005 |
| 2633 | 749.33 | 39.73 | 2 | 1497.65 | 1.48 | CountDiff |
| 2725 | 786.37 | 59.71 | 1 | 786.37 | 1.49 | P < 0.005 |
| 2932 | 920.45 | 44.92 | 1 | 920.45 | 1.49 | P < 0.005 |
| 2317 | 657.35 | 31.91 | 1 | 657.35 | 1.49 | P < 0.005 |
| 2618 | 744.34 | 53.76 | 3 | 2231 | 1.49 | P < 0.01 |
| 1074 | 463.58 | 47.03 | 5 | 2313.87 | 1.49 | P < 0.01 |
| 3459 | 593.49 | 56.69 | 5 | 2963.42 | 1.49 | P < 0.05 |
| 356 | 370.86 | 50.12 | 3 | 1110.56 | 1.5 | P < 0.001 |
| 2697 | 772.04 | 46.84 | 3 | 2314.1 | 1.5 | P < 0.001 |
| 4086 | 1061.58 | 52.2 | 1 | 1061.58 | 1.5 | P < 0.001 |
| 3852 | 799.11 | 66.21 | 4 | 3193.42 | 1.5 | P < 0.005 |
| 4524 | 852.86 | 55.21 | 4 | 3408.42 | 1.5 | P < 0.05 |
| 2171 | 628.71 | 51.69 | 1 | 628.71 | 1.5 | P < 0.05 |
| 3434 | 579.78 | 75.84 | 4 | 2316.1 | 1.5 | P < 0.05 |
| 80 | 319.68 | 48.65 | 4 | 1275.7 | 1.5 | P < 0.05 |
| 1258 | 487.26 | 45.19 | 2 | 973.51 | 1.51 | P < 0.001 |
| 2033 | 602.98 | 49.55 | 3 | 1806.92 | 1.51 | P < 0.005 |
| 1163 | 473.75 | 40.24 | 2 | 946.49 | 1.51 | P < 0.005 |
| 3068 | 1116.01 | 53.76 | 2 | 2231.01 | 1.51 | P < 0.01 |
| 2818 | 835.39 | 71.39 | 4 | 3338.54 | 1.51 | P < 0.05 |
| 4122 | 1162.85 | 73.85 | 3 | 3486.53 | 1.51 | P < 0.05 |
| 1276 | 489.73 | 50.37 | 2 | 978.45 | 1.51 | P < 0.05 |
| 2391 | 682.49 | 55.25 | 5 | 3408.42 | 1.52 | P < 0.05 |
| 365 | 372.72 | 40.29 | 1 | 372.72 | 1.52 | P < 0.05 |
| 2592 | 738.79 | 55.32 | 2 | 1476.57 | 1.53 | P < 0.05 |
| 3832 | 787.87 | 54.62 | 4 | 3148.46 | 1.53 | P < 0.05 |
| 4782 | 589.83 | 31.56 | 2 | 1178.65 | 1.53 | CountDiff |
| 1248 | 485.25 | 49.14 | 2 | 969.49 | 1.54 | P < 0.001 |
| 4123 | 1163.49 | 64.07 | 4 | 4650.94 | 1.54 | P < 0.005 |
| 823 | 432.74 | 35.99 | 2 | 864.47 | 1.54 | P < 0.01 |
| 3366 | 536.74 | 98.24 | 4 | 2143.94 | 1.54 | P < 0.05 |
| 218 | 347.68 | 26.64 | 2 | 694.35 | 1.55 | P < 0.05 |
| 3001 | 989.19 | 66.15 | 5 | 4941.92 | 1.55 | P < 0.05 |
| 331 | 366.41 | 46.86 | 4 | 1462.62 | 1.55 | P < 0.05 |
| 4632 | 930.97 | 64.08 | 5 | 4650.82 | 1.55 | P < 0.05 |
| 531 | 397.2 | 34.11 | 2 | 793.39 | 1.56 | P < 0.05 |
| 1301 | 492.88 | 55.24 | 3 | 1476.62 | 1.57 | P < 0.05 |
| 3649 | 680.08 | 81.09 | 4 | 2717.3 | 1.57 | P < 0.05 |
| 930 | 444.87 | 22.84 | 3 | 1332.59 | 1.57 | P < 0.05 |
| 4608 | 671.3 | 54.62 | 1 | 671.3 | 1.57 | CountDiff |
| 3726 | 725.33 | 57.49 | 1 | 725.33 | 1.58 | P < 0.001 |
| 4 | 300.7 | 26.26 | 2 | 600.39 | 1.58 | P < 0.005 |
| 563 | 401.49 | 39.18 | 3 | 1202.45 | 1.58 | P < 0.01 |
| 1537 | 526.29 | 62.08 | 3 | 1576.85 | 1.58 | P < 0.01 |
| 2411 | 688.37 | 25.86 | 1 | 688.37 | 1.59 | P < 0.001 |
| 1732 | 559.25 | 53.19 | 5 | 2792.22 | 1.59 | P < 0.05 |
| 1697 | 554.26 | 52.23 | 3 | 1660.76 | 1.59 | P < 0.05 |
| 2676 | 763.09 | 52.63 | 4 | 3049.34 | 1.6 | P < 0.005 |
| 114 | 328.2 | 31.63 | 1 | 328.2 | 1.6 | P < 0.05 |
| 1119 | 469.72 | 31.01 | 2 | 938.43 | 1.6 | P < 0.05 |
| 3224 | 456.25 | 23.34 | 2 | 911.49 | 1.6 | CountDiff |
| 992 | 451.98 | 49.51 | 4 | 1804.9 | 1.61 | P < 0.001 |
| 2894 | 883.89 | 52.24 | 2 | 1766.77 | 1.61 | P < 0.05 |
| 3435 | 579.78 | 73.1 | 4 | 2316.1 | 1.61 | P < 0.05 |
| 1848 | 573.57 | 50.13 | 5 | 2863.82 | 1.62 | P < 0.001 |
| 4259 | 455.53 | 41.8 | 3 | 1364.57 | 1.62 | P < 0.001 |
| 1316 | 495.27 | 40.48 | 2 | 989.53 | 1.62 | P < 0.001 |
| 3129 | 311.19 | 33.16 | 2 | 621.37 | 1.62 | P < 0.005 |
| 2356 | 671.34 | 38.28 | 1 | 671.34 | 1.62 | P < 0.005 |
| 2435 | 694.34 | 58.61 | 3 | 2081 | 1.62 | P < 0.01 |
| 3186 | 417.21 | 36.27 | 2 | 833.41 | 1.63 | P < 0.001 |
| 2024 | 601.75 | 39.19 | 2 | 1202.49 | 1.63 | P < 0.005 |
| 3650 | 680.08 | 82.94 | 4 | 2717.3 | 1.63 | P < 0.005 |
| 3084 | 1148.98 | 64.5 | 4 | 4592.9 | 1.63 | P < 0.005 |
| 3182 | 415.24 | 16 | 2 | 829.47 | 1.63 | P < 0.01 |
| 1302 | 492.94 | 57.64 | 3 | 1476.8 | 1.63 | P < 0.05 |
| 1971 | 594.06 | 48.62 | 1 | 594.06 | 1.63 | P < 0.05 |
| 152 | 336.17 | 38.28 | 2 | 671.33 | 1.64 | P < 0.005 |
| 3859 | 800.94 | 62.17 | 4 | 3200.74 | 1.64 | CountDiff |
| 1706 | 555.8 | 52.58 | 2 | 1110.59 | 1.65 | P < 0.001 |
| 4228 | 418.72 | 27.67 | 1 | 418.72 | 1.65 | P < 0.05 |
| 281 | 358.71 | 34.21 | 2 | 716.41 | 1.66 | P < 0.001 |
| 2930 | 919.39 | 64.5 | 5 | 4592.92 | 1.66 | P < 0.005 |
| 4664 | 608.94 | 54.61 | 3 | 1824.8 | 1.66 | P < 0.05 |
| 722 | 423.02 | 30.9 | 1 | 423.02 | 1.67 | P < 0.001 |
| 2073 | 610.66 | 52.64 | 5 | 3049.27 | 1.67 | P < 0.005 |
| 3215 | 444.27 | 40.66 | 1 | 444.27 | 1.67 | P < 0.01 |
| 4318 | 508.47 | 39.22 | 4 | 2030.86 | 1.67 | CountDiff |
| 4708 | 645.8 | 46.29 | 4 | 2580.18 | 1.67 | CountDiff |
| 2948 | 932.18 | 64.43 | 5 | 4656.87 | 1.7 | P < 0.05 |
| 1885 | 579.28 | 46.85 | 4 | 2314.1 | 1.71 | P < 0.001 |
| 2286 | 650.81 | 27.69 | 2 | 1300.61 | 1.71 | P < 0.001 |
| 3892 | 826.39 | 73.75 | 2 | 1651.77 | 1.71 | P < 0.01 |
| 3951 | 878.67 | 26.95 | 3 | 2633.99 | 1.71 | P < 0.05 |
| 411 | 381.18 | 24.76 | 2 | 761.35 | 1.71 | P < 0.05 |
| 4307 | 493.95 | 49.84 | 4 | 1972.78 | 1.71 | CountDiff |
| 2763 | 802.75 | 48.73 | 3 | 2406.23 | 1.72 | P < 0.005 |
| 4475 | 681.51 | 73.68 | 4 | 2723.02 | 1.72 | P < 0.05 |
| 1033 | 458.74 | 39.69 | 2 | 916.47 | 1.72 | P < 0.05 |
| 2866 | 858.48 | 35.02 | 1 | 858.48 | 1.74 | P < 0.001 |
| 4575 | 442.72 | 39.02 | 4 | 1767.86 | 1.75 | P < 0.01 |
| 4295 | 484.76 | 46.41 | 2 | 968.51 | 1.76 | P < 0.05 |
| 3566 | 639.49 | 66.22 | 5 | 3193.42 | 1.77 | P < 0.001 |
| 2069 | 609.79 | 71.58 | 2 | 1218.57 | 1.77 | P < 0.001 |
| 462 | 387.21 | 26.79 | 2 | 773.41 | 1.78 | P < 0.001 |
| 1784 | 565.27 | 79.16 | 4 | 2258.06 | 1.78 | P < 0.001 |
| 1487 | 519.74 | 46.69 | 2 | 1038.47 | 1.83 | P < 0.05 |
| 15 | 304.5 | 23.36 | 3 | 911.48 | 1.85 | P < 0.05 |
| 967 | 448.75 | 39.11 | 2 | 896.49 | 1.88 | P < 0.05 |
| 3617 | 663.26 | 67.78 | 4 | 2650.02 | 1.89 | P < 0.05 |
| 2887 | 878.41 | 56.31 | 2 | 1755.81 | 1.89 | NA |
| 1841 | 573.24 | 50.05 | 5 | 2862.17 | 1.92 | P < 0.05 |
| 2189 | 631.96 | 59.7 | 3 | 1893.86 | 1.93 | P < 0.01 |
| 3138 | 330.18 | 30.36 | 2 | 659.35 | 1.93 | P < 0.05 |
| 2616 | 744.34 | 62.03 | 3 | 2231 | 1.96 | P < 0.01 |
| 507 | 393.55 | 31.7 | 3 | 1178.63 | 2.03 | P < 0.01 |
| 4386 | 583.08 | 52.02 | 5 | 2911.37 | 2.05 | P < 0.01 |
| 2076 | 611.31 | 25.89 | 1 | 611.31 | 2.08 | CountDiff |
| 3066 | 1110.59 | 50.12 | 1 | 1110.59 | 2.1 | P < 0.001 |

TABLE 6
Unidentified Proteins Decreased in Subjects having RA

| Component | | R.T. | | | Fold | |
|---|---|---|---|---|---|---|
| # | m/z | (min.) | z | M + H | Change | P |
| 318 | 364.95 | 23.48 | 5 | 1820.72 | −1 | CountDiff |
| 1241 | 484.26 | 54.27 | 2 | 967.51 | −1.01 | CountDiff |
| 3903 | 837.4 | 27.47 | 1 | 837.4 | −1.01 | CountDiff |
| 83 | 320.78 | 31.6 | 3 | 960.32 | −1.02 | CountDiff |
| 3661 | 689.29 | 60.49 | 2 | 1377.57 | −1.06 | CountDiff |
| 3690 | 700.02 | 64.51 | 3 | 2098.04 | −1.08 | CountDiff |
| 3932 | 857.36 | 47.86 | 1 | 857.36 | −1.08 | CountDiff |
| 4115 | 1131.12 | 57.28 | 3 | 3391.34 | −1.08 | CountDiff |
| 551 | 400.19 | 15.89 | 2 | 799.37 | −1.09 | CountDiff |
| 4071 | 1025.1 | 59.01 | 3 | 3073.28 | −1.09 | CountDiff |
| 2504 | 710.85 | 47.1 | 1 | 710.85 | −1.1 | P < 0.01 |
| 4747 | 1116.4 | 39.7 | 5 | 5577.97 | −1.1 | CountDiff |
| 3230 | 459.86 | 60.4 | 3 | 1377.56 | −1.1 | CountDiff |
| 1139 | 471.73 | 42.23 | 2 | 942.45 | −1.11 | P < 0.05 |
| 4025 | 958.73 | 90.27 | 3 | 2874.17 | −1.11 | CountDiff |
| 2653 | 754.33 | 68.05 | 3 | 2260.97 | −1.12 | CountDiff |
| 602 | 407.26 | 42.79 | 1 | 407.26 | −1.13 | P < 0.01 |
| 3677 | 695.01 | 68.99 | 3 | 2083.01 | −1.13 | CountDiff |
| 3599 | 655.95 | 58.61 | 3 | 1965.83 | −1.13 | CountDiff |
| 2209 | 634.34 | 67.94 | 3 | 1901 | −1.13 | CountDiff |
| 756 | 426.2 | 46.65 | 3 | 1276.58 | −1.14 | P < 0.01 |
| 2410 | 687.82 | 51.52 | 2 | 1374.63 | −1.14 | P < 0.05 |
| 4446 | 638.34 | 62.21 | 3 | 1913 | −1.14 | CountDiff |
| 274 | 357.18 | 27.64 | 2 | 713.35 | −1.15 | P < 0.05 |
| 3161 | 394.68 | 42.76 | 1 | 394.68 | −1.15 | P < 0.05 |
| 4743 | 656.85 | 60.16 | 2 | 1312.69 | −1.15 | CountDiff |
| 4132 | 1186.96 | 69.82 | 4 | 4744.82 | −1.16 | P < 0.005 |
| 2014 | 600.28 | 44.19 | 1 | 600.28 | −1.16 | P < 0.01 |
| 2167 | 628.31 | 42.75 | 2 | 1255.61 | −1.16 | P < 0.05 |
| 1005 | 453.26 | 55.38 | 1 | 453.26 | −1.16 | CountDiff |
| 209 | 346.13 | 47.08 | 3 | 1036.37 | −1.17 | P < 0.01 |
| 1833 | 572.73 | 36.07 | 1 | 572.73 | −1.17 | P < 0.05 |
| 896 | 441.17 | 47.81 | 3 | 1321.49 | −1.18 | P < 0.005 |
| 3637 | 670.83 | 56.54 | 2 | 1340.65 | −1.18 | P < 0.05 |
| 1791 | 566.28 | 63.12 | 3 | 1696.82 | −1.18 | P < 0.05 |
| 3352 | 529.91 | 34.65 | 3 | 1587.71 | −1.18 | P < 0.05 |
| 3319 | 514.72 | 44.77 | 4 | 2055.86 | −1.18 | P < 0.05 |
| 3547 | 632.27 | 60.49 | 4 | 2526.06 | −1.18 | CountDiff |
| 2690 | 769.43 | 34.83 | 1 | 769.43 | −1.18 | CountDiff |
| 1553 | 528.46 | 60.79 | 4 | 2110.82 | −1.19 | P < 0.01 |
| 1622 | 540.27 | 52.88 | 3 | 1618.79 | −1.19 | P < 0.01 |
| 2041 | 605.28 | 60.14 | 2 | 1209.55 | −1.19 | CountDiff |
| 3185 | 416.25 | 29.14 | 1 | 416.25 | −1.2 | P < 0.001 |
| 781 | 429.21 | 45.22 | 3 | 1285.61 | −1.2 | P < 0.01 |
| 3743 | 737.28 | 60.1 | 5 | 3682.37 | −1.2 | P < 0.01 |
| 639 | 411.83 | 44.17 | 3 | 1233.47 | −1.2 | P < 0.05 |
| 1888 | 579.7 | 10.11 | 2 | 1158.39 | −1.2 | P < 0.05 |
| 1616 | 539.52 | 53.43 | 3 | 1616.54 | −1.2 | P < 0.05 |
| 3814 | 778.32 | 58.9 | 2 | 1555.63 | −1.2 | P < 0.05 |
| 705 | 420.2 | 46.68 | 3 | 1258.58 | −1.21 | P < 0.001 |
| 2242 | 638.81 | 46.68 | 2 | 1276.61 | −1.21 | P < 0.005 |
| 361 | 372.17 | 53.7 | 2 | 743.33 | −1.21 | P < 0.01 |
| 1684 | 551.26 | 62.19 | 3 | 1651.76 | −1.21 | P < 0.05 |
| 2644 | 752.77 | 29.66 | 2 | 1504.53 | −1.21 | P < 0.05 |
| 3653 | 682.8 | 66.48 | 2 | 1364.59 | −1.21 | P < 0.05 |
| 2476 | 704.3 | 60.83 | 3 | 2110.88 | −1.21 | P < 0.05 |
| 3302 | 503.27 | 62.28 | 2 | 1005.53 | −1.21 | CountDiff |
| 822 | 432.54 | 41.74 | 3 | 1295.6 | −1.22 | P < 0.001 |
| 933 | 445.22 | 25.58 | 2 | 889.43 | −1.22 | P < 0.05 |
| 3196 | 422.24 | 41.17 | 2 | 843.47 | −1.22 | P < 0.05 |
| 3282 | 497.2 | 26.65 | 2 | 993.39 | −1.22 | P < 0.05 |
| 1037 | 459.26 | 16.88 | 1 | 459.26 | −1.23 | P < 0.001 |
| 1526 | 524.78 | 52.11 | 2 | 1048.55 | −1.23 | P < 0.005 |
| 412 | 381.2 | 37.13 | 2 | 761.39 | −1.23 | P < 0.05 |
| 1886 | 579.3 | 39.98 | 1 | 579.3 | −1.23 | CountDiff |
| 1803 | 568.34 | 28.91 | 1 | 568.34 | −1.24 | P < 0.05 |
| 2321 | 659.29 | 38.31 | 2 | 1317.57 | −1.24 | P < 0.05 |
| 2150 | 625.79 | 51.25 | 2 | 1250.57 | −1.25 | P < 0.005 |
| 2454 | 698.96 | 60.79 | 3 | 2094.86 | −1.25 | P < 0.05 |
| 30 | 308.65 | 23.12 | 2 | 616.29 | −1.25 | P < 0.05 |
| 3786 | 758.81 | 61.48 | 4 | 3032.22 | −1.25 | P < 0.05 |
| 708 | 420.69 | 39.39 | 2 | 840.37 | −1.26 | P < 0.005 |
| 3528 | 627.76 | 31.87 | 2 | 1254.51 | −1.26 | P < 0.05 |
| 2627 | 746.39 | 54.08 | 2 | 1491.77 | −1.26 | P < 0.05 |
| 4315 | 504.3 | 59.03 | 1 | 504.3 | −1.26 | CountDiff |
| 3847 | 796.32 | 63.29 | 2 | 1591.63 | −1.27 | P < 0.01 |
| 3993 | 926.85 | 60.09 | 4 | 3704.38 | −1.27 | P < 0.01 |
| 3559 | 636.29 | 50.39 | 2 | 1271.57 | −1.27 | P < 0.05 |
| 1865 | 575.79 | 48.26 | 2 | 1150.57 | −1.27 | P < 0.05 |
| 2346 | 669.35 | 52.81 | 1 | 669.35 | −1.27 | CountDiff |
| 4720 | 868.39 | 95.83 | 3 | 2603.15 | −1.28 | P < 0.01 |
| 1339 | 499.17 | 30.18 | 3 | 1495.49 | −1.28 | P < 0.05 |
| 544 | 399.5 | 29.97 | 3 | 1196.48 | −1.28 | P < 0.05 |
| 3093 | 1181.18 | 69.83 | 4 | 4721.7 | −1.28 | P < 0.05 |
| 3231 | 460.86 | 66.48 | 3 | 1380.56 | −1.29 | P < 0.001 |
| 1523 | 524.53 | 58.91 | 3 | 1571.57 | −1.29 | P < 0.001 |
| 1343 | 499.71 | 41.52 | 4 | 1995.82 | −1.29 | P < 0.001 |
| 1582 | 533.92 | 72.35 | 3 | 1599.74 | −1.29 | P < 0.005 |
| 956 | 447.55 | 56.59 | 3 | 1340.63 | −1.29 | P < 0.01 |
| 2228 | 637.29 | 57.29 | 3 | 1909.85 | −1.29 | P < 0.05 |
| 569 | 402.18 | 39.56 | 4 | 1605.7 | −1.29 | P < 0.05 |
| 3460 | 593.86 | 46.52 | 3 | 1779.56 | −1.29 | P < 0.05 |
| 734 | 424.2 | 27.4 | 2 | 847.39 | −1.29 | NA |
| 3247 | 471.16 | 36.96 | 3 | 1411.46 | −1.3 | P < 0.005 |
| 3385 | 554.64 | 49.15 | 3 | 1661.9 | −1.3 | P < 0.005 |
| 2207 | 634.28 | 52.68 | 3 | 1900.82 | −1.3 | P < 0.005 |
| 1471 | 518.28 | 55.64 | 2 | 1035.55 | −1.3 | P < 0.01 |
| 3513 | 620.49 | 38 | 4 | 2478.94 | −1.3 | P < 0.01 |
| 2601 | 739.98 | 67.12 | 3 | 2217.92 | −1.3 | P < 0.05 |
| 1388 | 505.74 | 46.01 | 2 | 1010.47 | −1.31 | P < 0.005 |
| 1676 | 549.26 | 52.42 | 2 | 1097.51 | −1.32 | P < 0.005 |
| 1416 | 510.6 | 62.03 | 3 | 1529.78 | −1.32 | P < 0.01 |
| 4393 | 588.07 | 60.25 | 5 | 2936.32 | −1.32 | CountDiff |
| 4273 | 467.51 | 49.21 | 3 | 1400.51 | −1.32 | CountDiff |
| 1949 | 589.9 | 46.06 | 3 | 1767.68 | −1.33 | P < 0.001 |
| 979 | 450.21 | 41.32 | 2 | 899.41 | −1.33 | P < 0.005 |
| 1619 | 539.94 | 50.88 | 3 | 1617.8 | −1.34 | P < 0.005 |
| 1083 | 465.18 | 60.43 | 3 | 1393.52 | −1.34 | P < 0.005 |
| 4268 | 463.71 | 31.67 | 1 | 463.71 | −1.34 | P < 0.005 |
| 948 | 446.57 | 45.93 | 3 | 1337.69 | −1.34 | P < 0.005 |
| 2492 | 709.28 | 60.78 | 1 | 709.28 | −1.34 | P < 0.01 |
| 623 | 409.65 | 11.88 | 2 | 818.29 | −1.34 | P < 0.05 |
| 3922 | 848.59 | 57.28 | 4 | 3391.34 | −1.34 | P < 0.05 |
| 922 | 444.2 | 46.5 | 4 | 1773.78 | −1.34 | P < 0.05 |
| 1893 | 580.27 | 37.89 | 2 | 1159.53 | −1.35 | P < 0.005 |
| 3375 | 543.28 | 41.64 | 2 | 1085.55 | −1.35 | P < 0.01 |
| 1459 | 516.76 | 36.71 | 2 | 1032.51 | −1.35 | P < 0.05 |
| 1219 | 480.95 | 54.37 | 4 | 1920.78 | −1.36 | P < 0.001 |
| 1501 | 521.5 | 69.02 | 4 | 2082.98 | −1.36 | P < 0.001 |
| 3028 | 1022.75 | 58.92 | 3 | 3066.23 | −1.36 | P < 0.05 |
| 3379 | 547.79 | 41.79 | 2 | 1094.57 | −1.36 | P < 0.05 |
| 2175 | 629.28 | 36.79 | 1 | 629.28 | −1.36 | P < 0.05 |
| 1480 | 519.21 | 58.92 | 3 | 1555.61 | −1.37 | P < 0.001 |
| 2386 | 681.32 | 59.85 | 1 | 681.32 | −1.37 | P < 0.01 |
| 1545 | 527.57 | 48.59 | 3 | 1580.69 | −1.37 | P < 0.05 |
| 2135 | 624.28 | 66.75 | 3 | 1870.82 | −1.37 | P < 0.05 |
| 1583 | 533.97 | 50.62 | 4 | 2132.86 | −1.38 | P < 0.01 |
| 468 | 387.71 | 36 | 4 | 1547.82 | −1.38 | P < 0.05 |
| 1500 | 521.32 | 49.35 | 1 | 521.32 | −1.39 | P < 0.05 |
| 2687 | 768.41 | 65.45 | 1 | 768.41 | −1.39 | P < 0.05 |
| 4507 | 745.7 | 38.35 | 2 | 1490.39 | −1.39 | P < 0.05 |
| 3813 | 777.73 | 29.64 | 2 | 1554.45 | −1.39 | P < 0.05 |
| 534 | 397.65 | 23.95 | 2 | 794.29 | −1.4 | P < 0.005 |
| 3916 | 845.86 | 64.65 | 2 | 1690.71 | −1.41 | P < 0.05 |
| 333 | 366.68 | 25.31 | 2 | 732.35 | −1.42 | P < 0.005 |
| 4233 | 421.68 | 62.22 | 4 | 1683.7 | −1.42 | P < 0.05 |
| 4522 | 842.36 | 62.23 | 2 | 1683.71 | −1.43 | P < 0.005 |
| 4715 | 729.06 | 70.36 | 4 | 2913.22 | −1.43 | P < 0.01 |
| 4515 | 797.8 | 58.98 | 2 | 1594.59 | −1.44 | P < 0.005 |
| 1623 | 540.27 | 51.25 | 3 | 1618.79 | −1.46 | P < 0.001 |
| 2412 | 688.74 | 34.24 | 2 | 1376.47 | −1.48 | P < 0.01 |
| 1682 | 550.32 | 46.09 | 1 | 550.32 | −1.48 | P < 0.05 |
| 4485 | 715.23 | 43.15 | 3 | 2143.67 | −1.48 | P < 0.05 |
| 2066 | 609.3 | 48.71 | 1 | 609.3 | −1.48 | P < 0.05 |
| 4451 | 644.3 | 44.82 | 3 | 1930.88 | −1.49 | P < 0.05 |
| 4454 | 646.63 | 57.7 | 3 | 1937.87 | −1.5 | P < 0.05 |
| 3152 | 356.5 | 55.62 | 3 | 1067.48 | −1.5 | P < 0.05 |
| 1028 | 458.22 | 33.59 | 1 | 458.22 | −1.51 | P < 0.001 |
| 3357 | 532.2 | 59.04 | 3 | 1594.58 | −1.51 | P < 0.05 |
| 351 | 369.71 | 35.73 | 2 | 738.41 | −1.52 | P < 0.05 |
| 2413 | 688.78 | 34.81 | 2 | 1376.55 | −1.53 | NA |
| 811 | 431.88 | 36.99 | 3 | 1293.62 | −1.54 | P < 0.05 |
| 1513 | 522.95 | 51.24 | 3 | 1566.83 | −1.55 | P < 0.01 |
| 1013 | 454.73 | 42.15 | 2 | 908.45 | −1.56 | P < 0.005 |
| 683 | 417.7 | 28.98 | 2 | 834.39 | −1.56 | P < 0.005 |
| 919 | 444.19 | 34.05 | 2 | 887.37 | −1.56 | P < 0.05 |
| 790 | 429.85 | 50.45 | 3 | 1287.53 | −1.57 | P < 0.05 |
| 3360 | 534.26 | 55.63 | 2 | 1067.51 | −1.58 | P < 0.05 |
| 2020 | 601.27 | 54.07 | 2 | 1201.53 | −1.58 | P < 0.05 |
| 2485 | 707.8 | 68.94 | 2 | 1414.59 | −1.59 | P < 0.05 |
| 246 | 352.14 | 42.14 | 3 | 1054.4 | −1.59 | P < 0.05 |
| 1208 | 480.23 | 52.23 | 2 | 959.45 | −1.59 | P < 0.05 |
| 1324 | 496.9 | 53.03 | 3 | 1488.68 | −1.59 | P < 0.05 |
| 1557 | 529.28 | 47.26 | 2 | 1057.55 | −1.61 | P < 0.005 |
| 606 | 407.7 | 29.3 | 2 | 814.39 | −1.61 | P < 0.05 |
| 4126 | 1176.92 | 73.99 | 4 | 4704.66 | −1.62 | P < 0.001 |
| 1179 | 475.74 | 51.59 | 2 | 950.47 | −1.63 | P < 0.05 |
| 1145 | 472.47 | 41.83 | 4 | 1886.86 | −1.66 | P < 0.05 |
| 2648 | 753.26 | 51.11 | 2 | 1505.51 | −1.7 | P < 0.005 |
| 891 | 440.21 | 53.63 | 2 | 879.41 | −1.71 | P < 0.005 |
| 3365 | 536.68 | 43.17 | 4 | 2143.7 | −1.73 | P < 0.05 |
| 4238 | 429.67 | 53.75 | 4 | 1715.66 | −1.75 | P < 0.005 |
| 1330 | 497.77 | 46.04 | 2 | 994.53 | −1.78 | CountDiff |
| 2341 | 668.33 | 54.87 | 1 | 668.33 | −1.83 | P < 0.001 |
| 147 | 334.66 | 54.92 | 2 | 668.31 | −1.83 | CountDiff |
| 1595 | 536.27 | 58.54 | 2 | 1071.53 | −1.85 | P < 0.005 |
| 276 | 357.2 | 31.12 | 2 | 713.39 | −1.85 | P < 0.05 |
| 4329 | 519.55 | 56.49 | 3 | 1556.63 | −1.86 | P < 0.05 |
| 989 | 451.69 | 31.31 | 2 | 902.37 | −1.89 | CountDiff |
| 4187 | 344.8 | 38.35 | 3 | 1032.38 | −1.95 | P < 0.05 |
| 4328 | 517.71 | 41.31 | 2 | 1034.41 | −1.96 | P < 0.05 |
| 3263 | 483.45 | 44.81 | 4 | 1930.78 | −1.99 | P < 0.05 |
| 3953 | 882.4 | 47.02 | 2 | 1763.79 | −2 | P < 0.01 |
| 1566 | 531.71 | 48.59 | 2 | 1062.41 | −2.02 | P < 0.001 |
| 4327 | 516.7 | 38.34 | 2 | 1032.39 | −2.03 | P < 0.05 |
| 3523 | 625.23 | 34.1 | 3 | 1873.67 | −2.04 | P < 0.005 |
| 625 | 409.71 | 35.09 | 2 | 818.41 | −2.05 | P < 0.001 |
| 2395 | 683.33 | 64.51 | 1 | 683.33 | −2.07 | P < 0.001 |
| 1097 | 466.75 | 48.7 | 2 | 932.49 | −2.08 | P < 0.005 |
| 189 | 342.16 | 64.49 | 2 | 683.31 | −2.13 | P < 0.005 |
| 484 | 389.22 | 34.35 | 2 | 777.43 | −2.13 | P < 0.01 |
| 4172 | 301.12 | 46.02 | 2 | 601.23 | −2.28 | P < 0.05 |
| 4179 | 323.14 | 48.38 | 2 | 645.27 | −2.32 | P < 0.01 |
| 2819 | 835.38 | 71.65 | 4 | 3338.5 | −2.32 | P < 0.05 |
| 373 | 373.74 | 75.12 | 2 | 746.47 | −2.47 | P < 0.001 |

TABLE 7
Cell Populations Increased in Subjects having RA

| | | | | | | | Effect |
|---|---|---|---|---|---|---|---|
| Study | General Cell Type | Assay | Cell Population | Property | p | adjp | Size |
| Study2 | B cell subset | CD69_CD71_CD20 | CD20pCD69p/CD20p | RATIO | 0.034 | 1 | 0.2562762 |
| Study 1 | B Cell subset | CD69_CD71_CD20v4 | CD20pCD71p/CD20p | RATIO | 0.000028 | 0.017111 | 0.9447959 |
| Study2 | CD4 T Cell subset | CD26_CD4_CD3 | CD3pCD4pCD26p/CD3pCD4p | RATIO | <0.001 | 0.594 | 0.7056072 |
| Study2 | CD4 T Cell subset | CD101_CD14_CD4 | CD4pCD14nCD101p | COUNT | <0.001 | 0.471 | 0.6725686 |
| Study2 | CD4 T Cell subset | CD101_CD14_CD4 | CD4pCD14nCD101p/CD4pCD14n | RATIO | <0.001 | 0.012 | 0.8145183 |
| Study2 | CD4 T Cell subset | CD25_CD14_CD4 | CD4pCD14nCD25p/CD4pCD14n | RATIO | 0.027 | 1 | 0.4226701 |
| Study2 | CD4 T Cell subset | CD38_CD14_CD4 | CD4pCD14nCD38p/CD4pCD14n | RATIO | 0.015 | 1 | 0.5438194 |
| Study2 | CD4 T Cell subset | CD71_CD14_CD4 | CD4pCD14nCD71p | COUNT | 0.004 | 1 | 0.4125228 |
| Study2 | CD4 T Cell subset | CD71_CD14_CD4 | CD4pCD14nCD71p/CD4pCD14n | RATIO | 0.002 | 1 | 0.3309689 |
| Study 1 | CD4 T Cell subset | CD45RB_CD27_CD4v3 | CD4pCD27pCD45RBp | COUNT | 0.009331 | 1.000000 | 0.4183194 |
| Study2 | CD4 T Cell subset | CD28_CD45RA_CD4 | CD4pCD28pCD45RAp/CD4p | RATIO | 0.006 | 1 | 0.5449754 |
| Study2 | CD4 T Cell subset | CD62L_CD45RA_CD4 | CD4pCD45RApCD62Lp/CD4p | RATIO | 0.022 | 1 | 0.4697498 |
| Study2 | CD4 T Cells | CD4_CD8_CD3 | CD4 T cells/T cells | RATIO | <0.001 | 0.027 | 0.8437971 |
| Study 1 | CD4 T Cells | AVERAGE | CD4 T cells/T cells | RATIO | 0.000002 | 0.001481 | 0.8474299 |
| Study2 | CD8 T Cell subset | CD57_CD6_CD8 | CD6pCD8pCD57p/CD6pCD8p | RATIO | 0.012 | 1 | 0.4859035 |
| Study2 | CD8 T Cell subset | CD26_CD7_CD8 | CD7pCD8pCD26p/CD7pCD8p | RATIO | 0.026 | 1 | 0.4544495 |
| Study2 | CD8 T Cell subset | CD38_CD20_CD8 | CD8pCD20nCD38p/CD8pCD20n | RATIO | 0.002 | 1 | 0.710085 |
| Study 1 | CD8 T cell subset | CD95_CD20_CD8v3 | CD8pCD20nCD95p/CD8pCD20n | RATIO | 0.037409 | 1.000000 | 0.3435766 |
| Study 1 | CD8 T cell subset | CD69_CD25_CD8v9 | CD8pCD25p/CD8p | RATIO | 0.011157 | 1.000000 | 0.4500325 |
| Study2 | CD8 T Cell subset | CD69_CD25_CD8 | CD8pCD25p/CD8p | RATIO | 0.018 | 1 | 0.5885806 |
| Study2 | CD8 T Cell subset | CD28_CD62L_CD8 | CD8pCD28nCD62Lp/CD8p | RATIO | 0.027 | 1 | 0.2799138 |
| Study2 | CD8 T Cell subset | CD28_CD62L_CD8 | CD8pCD28pCD62Lp/CD8p | RATIO | 0.023 | 1 | 0.3682324 |
| Study2 | CD8 T Cell subset | CD161_CD45RA_CD8 | CD8pCD45RApCD161p/CD8p | RATIO | 0.034 | 1 | 0.3000852 |
| Study2 | CD8 T Cell subset | CD60_CD45RA_CD8 | CD8pCD45RApCD60p/CD8p | RATIO | 0.046 | 1 | 0.3834613 |
| Study2 | CD8 T Cell subset | CD62L_CD45RA_CD8 | CD8pCD45RApCD62Lp/CD8p | RATIO | 0.023 | 1 | 0.5056861 |
| Study 1 | CD8 T cell subset | CD71_CD57_CD8v7 | CD8pCD57p/CD8p | RATIO | 0.023666 | 1.000000 | 0.4121008 |
| Study2 | CD8 T Cell subset | CD69_CD25_CD8 | CD8pCD69p/CD8p | RATIO | 0.004 | 1 | 0.5096072 |
| Study2 | CD8 T Cell subset | CD71_CD57_CD8 | CD8pCD71p/CD8p | RATIO | <0.001 | 0.225 | 0.5837997 |
| Study 1 | Eosinophils | AVERAGE | Eosinophils | COUNT | 0.049423 | 1.000000 | 0.2972259 |
| Study 1 | Ganulocytes | AVERAGE | Granulocytes/WBC | RATIO | 0.002313 | 1.000000 | 0.5760901 |
| Study 1 | Ganulocytes subset | CD52_CD66b_CD16v10 | CD16pCD66bpCD52n | COUNT | 0.000839 | 0.490015 | 0.6358233 |
| Study 1 | Granulocyet subset | CD89_CD15_CD14v13 | CD14nCD15pCD89p | COUNT | 0.010515 | 1.000000 | 0.4998616 |
| Study2 | Granulocyte | CD45_CD14_CD16 | CD14nCD16pCD45p/CD45p | RATIO | <0.001 | 0.159 | 0.6611517 |
| Study2 | Granulocyte subset | CD32_CD11b_CD16 | CD11bpCD16p | COUNT | <0.001 | 0.522 | 0.6897164 |
| Study2 | Granulocyte subset | CD32_CD11b_CD16 | CD11bpCD16pCD32p | COUNT | <0.001 | 0.314 | 0.7209454 |
| Study2 | Granulocyte subset | CD89_CD15_CD14 | CD14nCD15pCD89p | COUNT | 0.004 | 1 | 0.606532 |
| Study 1 | Granulocyte subset | CD64_CD14_CD16v11 | CD14nCD16pCD64n | COUNT | 0.004652 | 1.000000 | 0.5420431 |
| Study2 | Granulocyte subset | CD44_CD18_CD16 | CD16pCD18pCD44p | COUNT | 0.002 | 0.832 | 0.662342 |
| Study 1 | Granulocytes | AVERAGE | Granulocytes | COUNT | 0.003999 | 1.000000 | 0.5622894 |
| Study2 | Granulocytes | CD45_CD14_CD16 | Granulocytes | COUNT | <0.001 | 0.417 | 0.6978234 |
| Study2 | Leukocytes | CD45_CD14_CD16 | CD45p | COUNT | 0.005 | 1 | 0.5891922 |
| Study 1 | Leukocytes | AVERAGE | WBC | COUNT | 0.022359 | 1.000000 | 0.4339659 |
| Study 1 | Monocyte subset | CCR5_CD60_CD14v9 | CCR5nCD14pCD60n | COUNT | 0.000018 | 0.011226 | 0.8008547 |
| Study2 | Monocyte subset | CD89_CD15_CD14 | CD14pCD15n | COUNT | 0.02 | 1 | 0.4712457 |
| Study2 | Monocyte subset | CD89_CD15_CD14 | CD14pCD15nCD89p | COUNT | 0.023 | 1 | 0.3803188 |
| Study 1 | Monocyte subset | CD89_CD15_CD14v13 | CD14pCD15nCD89p | COUNT | 0.000067 | 0.040736 | 0.7798395 |
| Study 1 | Monocyte subset | CD119_CD14_CD16v6 | CD14pCD16nCD119n | COUNT | 0.002333 | 1.000000 | 0.5632859 |
| Study2 | Monocyte subset | CD64_CD14_CD16 | CD14pCD16nCD64p | COUNT | 0.033 | 1 | 0.4669147 |
| Study2 | Monocyte subset | CD40_CD14_CD20 | CD14pCD20nCD40n/CD14pCD20n | RATIO | 0.003 | 1 | 0.6391118 |
| Study 1 | Monocyte subset | CD62L_CD14_CD20v7 | CD14pCD20nCD62Ln | COUNT | 0.000349 | 0.206385 | 0.7185618 |
| Study2 | Monocyte subset | HLADP_CD14_CD20 | CD14pCD20nDPn | COUNT | 0.006 | 1 | 0.5451047 |
| Study2 | Monocyte subset | HLADQ_CD14_CD20 | CD14pCD20nDQn | COUNT | <0.001 | 0.199 | 0.8248779 |
| Study2 | Monocyte subset | HLADQ_CD14_CD20 | CD14pCD20nDQt | COUNT | 0.017 | 1 | 0.4817503 |
| Study2 | Monocyte subset | HLADR4_CD14_CD20 | CD14pCD20nDR4n | COUNT | <0.001 | 0.214 | 0.6198077 |
| Study2 | Monocyte subset | HLADR_CD14_CD20 | CD14pCD20nDRn | COUNT | <0.001 | 0.706 | 0.5486261 |
| Study 1 | Monocyte subset | HLADP_CD14_CD20v8 | CD14pCD20nHLADPn | COUNT | 0.000496 | 0.290933 | 0.7591109 |
| Study 1 | Monocyte subset | HLADQ_CD14_CD20v8 | CD14pCD20nHLADQn | COUNT | 0.001188 | 0.686415 | 0.7638421 |
| Study 1 | Monocyte subset | HLADR_CD14_CD20v9 | CD14pCD20nHLADRp | COUNT | 0.000187 | 0.111461 | 0.6868374 |
| Study 1 | Monocyte subset | HLAPAN_CD14_CD20v7 | CD14pCD20nHLAPANp | COUNT | 0.000012 | 0.007617 | 0.8128984 |
| Study2 | Monocyte subset | HLAPAN_CD14_CD20 | CD14pCD20nPANt | COUNT | 0.044 | 1 | 0.4641527 |
| Study2 | Monocyte subset | CCR5_CD60_CD14 | CD14pCD60n | COUNT | 0.014 | 1 | 0.5367433 |
| Study 1 | Monocyte subset | TLR4_CD33_CD20v2 | CD20nCD33pTLR4p/CD20nCD33p | RATIO | 0.001021 | 0.593409 | 0.3967629 |
| Study 1 | Monocyte subset | CD54_CD14_CD3v10 | CD3nCD14pCD54n | COUNT | 0.005090 | 1.000000 | 0.5809295 |
| Study 1 | Monocyte subset | CD54_CD14_CD3v10 | CD3nCD14pCD54p | COUNT | 0.036752 | 1.000000 | 0.4551894 |
| Study2 | Monocyte subset | CD26_CD4_CD3 | CD3pCD4pCD26p | COUNT | 0.015 | 1 | 0.2978743 |
| Study 1 | Monocyte subset | CD101_CD14_CD4v3 | CD4pnCD14pCD101n | COUNT | 0.000073 | 0.043861 | 0.7771187 |
| Study2 | Monocyte subset | CD33_CD14_CD4 | CD4pnCD14pCD33p | COUNT | 0.02 | 1 | 0.4861742 |
| Study 1 | Monocyte subset | CD38_CD14_CD4v10 | CD4pnCD14pCD38n | COUNT | 0.044678 | 1.000000 | 0.4373296 |
| Study 1 | Monocyte subset | CD38_CD14_CD4v10 | CD4pnCD14pCD38p | COUNT | 0.015898 | 1.000000 | 0.5011491 |
| Study 1 | Monocyte subset | CD86_CD14_CD4v6 | CD4pnCD14pCD86n | COUNT | 0.000042 | 0.025753 | 0.784163 |
| Study2 | Monocyte subset | CD95_CD4_CD14 | CD4pnCD14pCD95t | COUNT | 0.014 | 1 | 0.572237 |
| Study2 | Monocytes | CD45_CD14_CD16 | CD14pCD16nCD45p | COUNT | 0.028 | 1 | 0.3914396 |
| Study 1 | Monocytes | CD64_CD14_CD16v11 | CD14pCD16nCD64p | COUNT | 0.000015 | 0.008968 | 0.8132311 |
| Study2 | Monocytes | CD40_CD14_CD20 | CD14pCD20nCD40n | COUNT | <0.001 | 0.301 | 0.7881695 |
| Study 1 | Monocytes | AVERAGE | Monocytes | COUNT | 0.000003 | 0.001611 | 0.8927377 |
| Study 1 | Monocytes | AVERAGE | Monocytes/WBC | RATIO | 0.000861 | 0.502040 | 0.6049538 |
| Study 1 | Neutrophils | CD32_CD11b_CD16v4 | CD11bpCD16n | COUNT | 0.000003 | 0.001626 | 0.5854824 |
| Study 1 | Neutrophils | AVERAGE | Neutrophils | COUNT | 0.012507 | 1.000000 | 0.4841396 |
| Study 1 | Neutrophils | AVERAGE | Neutrophils/WBC | RATIO | 0.010949 | 1.000000 | 0.3581328 |
| Study 1 | NK cell subset | CD161_CD56_CD3v2 | CD3nCD56pCD161n | COUNT | 0.002574 | 1.000000 | 0.5537043 |
| Study 1 | Other | CD52_CD66b_CD16v10 | CD16nCD66bpCD52p | COUNT | 0.039894 | 1.000000 | 0.423507 |
| Study2 | T cell subset | TCRab_TCRgd_CD3 | CD3pTCRabp/CD3pTCRgdp | RATIO | 0.031 | 1 | 0.2495411 |
| Study 1 | T Cell subset | AVERAGE | CD4 T cells/CD8 T cells | RATIO | 0.000000 | 0.000125 | 0.9125348 |
| Study2 | T Cells subset | CD4_CD8_CD3 | CD3pCD4p/CD3pCD8p | RATIO | <0.001 | 0.011 | 0.739175 |

TABLE 8
Cell Populations Decreased in Subjects having RA

| | | | | | | | Effect |
|---|---|---|---|---|---|---|---|
| Study | General Cell Type | Assay | Cell Population | Property | P | adjp | Size |
| Study 1 | B cell subset | CD38_CD20_CD8v8 | CD8nCD20pCD38p | COUNT | 0.002862 | 1.000000 | 0.3682915 |
| Study 1 | B cell subset | CD62L_CD14_CD20v7 | CD14nCD20pCD62Lp | COUNT | 0.028677 | 1.000000 | 0.3458029 |
| Study2 | B cell subset | CD95_CD20_CD8 | CD8nCD20pCD95p/ | RATIO | 0.044 | 1 | 0.5172224 |
| | | | CD8nCD20p | | | | |
| Study 1 | CD4 T Cell subset | CCR5_CD60_CD4v8 | CCR5pCD4pCD60n | COUNT | 0.039289 | 1.000000 | 0.143363 |
| Study2 | CD4 T Cell subset | CCR5_CD60_CD4 | CCR5pCD4pCD60n | COUNT | <0.001 | 0.484 | 0.6594824 |
| Study2 | CD4 T Cell subset | CCR5_CD60_CD4 | CCR5pCD4pCD60p | COUNT | 0.02 | 1 | 0.6180808 |
| Study 1 | CD4 T Cell subset | CCR5_CD60_CD4v8 | CCR5pCD4pCD60p | COUNT | 0.000003 | 0.001971 | 0.8299302 |
| Study2 | CD4 T Cell subset | CCR5_CD60_CD4 | CCR5pCD4pCD60p/ | RATIO | 0.044 | 1 | 0.5271522 |
| | | | CD4pCD60p | | | | |
| Study2 | CD4 T Cell subset | CD26_CD4_CD3 | CD3pCD4nCD26p | COUNT | <0.001 | 0.582 | 0.6011615 |
| Study2 | CD4 T Cell subset | CD71_CD14_CD4 | CD4pCD14nCD71n | INTENSITY2 | 0.037 | 1 | 0.2079245 |
| Study 1 | CD4 T Cell subset | CD45RB_CD27_CD4v3 | CD4pCD27nCD45RBpn | COUNT | 0.000339 | 0.201163 | 0.6604196 |
| Study2 | CD4 T Cell subset | CD28_CD45RA_CD4 | CD4pCD28nCD45RAn | COUNT | 0.024 | 1 | 0.5365788 |
| Study 1 | CD4 T Cell subset | CD62L_CD45RA_CD4v10 | CD4pCD45RAnCD62Ln | COUNT | 0.020345 | 1.000000 | 0.2301247 |
| Study2 | CD4 T Cell subset | CD62L_CD45RA_CD4 | CD4pCD45RAnCD62Ln | COUNT | 0.003 | 1 | 0.5810947 |
| Study2 | CD4 T Cell subset | CCR5_CD60_CD4 | CD4pCD60n | COUNT | 0.005 | 1 | 0.3887799 |
| Study 1 | CD8 T cell subset | CCR5_CD60_CD8v10 | CCR5nCD8pCD60n | COUNT | 0.000340 | 0.201163 | 0.5948072 |
| Study 1 | CD8 T cell subset | CCR5_CD60_CD8v10 | CCR5pCD8nCD60p | COUNT | 0.000154 | 0.091844 | 0.648314 |
| Study 1 | CD8 T cell subset | CCR5_CD60_CD8v10 | CCR5pCD8pCD60n | COUNT | 0.000220 | 0.131181 | 0.5630113 |
| Study2 | CD8 T Cell subset | CCR5_CD60_CD8 | CCR5pCD8pCD60n | COUNT | <0.001 | 0.55 | 0.6738924 |
| Study 1 | CD8 T cell subset | CCR5_CD60_CD8v10 | CCR5pCD8pCD60p | COUNT | 0.000083 | 0.049881 | 0.6799198 |
| Study 1 | CD8 T cell subset | CD57_CD6_CD8v7 | CD6pCD8pCD57n | COUNT | 0.000021 | 0.013126 | 0.7616691 |
| Study 1 | CD8 T cell subset | CD57_CD6_CD8v7 | CD6pCD8pCD57p | COUNT | 0.034692 | 1.000000 | 0.2409203 |
| Study 1 | CD8 T cell subset | CD26_CD7_CD8v6 | CD7pCD8pCD26n | COUNT | 0.000040 | 0.024211 | 0.6693274 |
| Study 1 | CD8 T cell subset | CD26_CD7_CD8v6 | CD7pCD8pCD26p | COUNT | 0.000007 | 0.004312 | 0.7044912 |
| Study2 | CD8 T Cell subset | CD26_CD7_CD8 | CD7pCD8pn | COUNT | 0.049 | 1 | 0.2474836 |
| Study 1 | CD8 T cell subset | CD26_CD7_CD8v6 | CD7pCD8pnCD26p | COUNT | 0.000000 | 0.000174 | 0.5913666 |
| Study 1 | CD8 T cell subset | CD101_CD8_CD16v4 | CD8pCD16nCD101n | COUNT | 0.000006 | 0.003846 | 0.7599001 |
| Study 1 | CD8 T cell subset | CD101_CD8_CD16v4 | CD8pCD16nCD101p | COUNT | 0.001030 | 0.597433 | 0.5640556 |
| Study2 | CD8 T Cell subset | CD101_CD8_CD16 | CD8pCD16nCD101p | COUNT | 0.018 | 1 | 0.5640679 |
| Study 1 | CD8 T cell subset | CD38_CD20_CD8v8 | CD8pCD20nCD38n | COUNT | 0.000083 | 0.049859 | 0.5750184 |
| Study 1 | CD8 T cell subset | CD38_CD20_CD8v8 | CD8pCD20nCD38p | COUNT | 0.002985 | 1.000000 | 0.6074203 |
| Study2 | CD8 T Cell subset | CD44_CD20_CD8 | CD8pCD20nCD44p/ | RATIO | 0.003 | 1 | 0.6407937 |
| | | | CD8pCD20n | | | | |
| Study2 | CD8 T Cell subset | CD95_CD20_CD8 | CD8pCD20nCD95p | COUNT | 0.002 | 1 | 0.5072614 |
| Study2 | CD8 T Cell subset | CD27_CD45RA_CD8 | CD8pCD27nCD45RAp | COUNT | 0.023 | 1 | 0.2520115 |
| Study2 | CD8 T Cell subset | CD27_CD45RA_CD8 | CD8pCD27pCD45RAp | COUNT | 0.024 | 1 | 0.2149937 |
| Study 1 | CD8 T cell subset | CD28_CD62L_CD8v12 | CD8pCD28nCD62Ln | COUNT | 0.002633 | 1.000000 | 0.4409627 |
| Study2 | CD8 T Cell subset | CD28_CD62L_CD8 | CD8pCD28pCD62Ln | COUNT | <0.001 | 0.554 | 0.6604842 |
| Study 1 | CD8 T cell subset | CD28_CD62L_CD8v12 | CD8pCD28pCD62Ln | COUNT | 0.000000 | 0.000270 | 1.1057356 |
| Study2 | CD8 T Cell subset | CD28_CD62L_CD8 | CD8pCD28pCD62Ln/ | RATIO | 0.02 | 1 | 0.3431341 |
| | | | CD8p | | | | |
| Study2 | CD8 T Cell subset | CD60_CD45RA_CD8 | CD8pCD45RAnCD60n | COUNT | <0.001 | 0.054 | 0.9521301 |
| Study 1 | CD8 T cell subset | CD62L_CD45RA_CD8v7 | CD8pCD45RAnCD62Ln | COUNT | 0.000000 | 0.000223 | 0.8592887 |
| Study2 | CD8 T Cell subset | CD62L_CD45RA_CD8 | CD8pCD45RAnCD62Ln | COUNT | <0.001 | 0.021 | 0.8769976 |
| Study 1 | CD8 T cell subset | CD62L_CD45RA_CD8v7 | CD8pCD45RAnCD62Lp | COUNT | 0.010010 | 1.000000 | 0.4030196 |
| Study2 | CD8 T Cell subset | CD161_CD45RA_CD8 | CD8pCD45RApCD161n | COUNT | 0.039 | 1 | 0.2853463 |
| Study2 | CD8 T Cell subset | CD60_CD45RA_CD8 | CD8pCD45RApCD60n | COUNT | 0.027 | 1 | 0.3142181 |
| Study 1 | CD8 T cell subset | CD62L_CD45RA_CD8v7 | CD8pCD45RApCD62Ln | COUNT | 0.001431 | 0.821474 | 0.3975255 |
| Study2 | CD8 T Cell subset | CD62L_CD45RA_CD8 | CD8pCD45RApCD62Ln | COUNT | 0.004 | 1 | 0.4458392 |
| Study 1 | CD8 T cell subset | CD62L_CD45RA_CD8v7 | CD8pCD45RApCD62Lp | COUNT | 0.000970 | 0.564536 | 0.6330695 |
| Study2 | CD8 T Cell subset | CD71_CD57_CD8 | CD8pCD57n | COUNT | <0.001 | 0.122 | 0.7908748 |
| Study 1 | CD8 T cell subset | CD94_CD57_CD8v2 | CD8pCD57nCD94n | COUNT | 0.000466 | 0.273881 | 0.7045932 |
| Study 1 | CD8 T cell subset | CD94_CD57_CD8v2 | CD8pCD57nCD94p | COUNT | 0.038480 | 1.000000 | 0.3735341 |
| Study2 | CD8 T Cell subset | CD71_CD57_CD8 | CD8pCD57p | COUNT | 0.003 | 1 | 0.6847291 |
| Study 1 | CD8 T cell subset | CD94_CD57_CD8v2 | CD8pCD57pCD94n | COUNT | 0.004381 | 1.000000 | 0.2096493 |
| Study2 | CD8 T Cell subset | CD69_CD25_CD8 | CD8pCD69n | COUNT | <0.001 | 0.161 | 0.7076249 |
| Study2 | CD8 T Cell subset | CD71_CD57_CD8 | CD8pCD71n | COUNT | <0.001 | 0.009 | 0.9965162 |
| Study2 | CD8 T Cells | AVERAGE | CD8 T cells | COUNT | <0.001 | 0.14 | 0.6295512 |
| Study 1 | CD8 T Cells | AVERAGE | CD8 T cells | COUNT | 0.000003 | 0.002003 | 0.8110482 |
| Study2 | CD8 T cells | CD4_CD8_CD3 | CD8 T cells/T cells | RATIO | <0.001 | 0.008 | 0.9017942 |
| Study 1 | CD8 T Cells | AVERAGE | CD8 T cells/T cells | RATIO | 0.000000 | 0.000151 | 1.037131 |
| Study 1 | CD8 T Cells | AVERAGE | CD8 T cells/WBC | RATIO | 0.000000 | 0.000011 | 1.1785306 |
| Study2 | Lymphocyte | CD45_CD14_CD16 | CD14nCD16nCD45p/ | RATIO | <0.001 | 0.125 | 0.6246031 |
| | | | CD45p | | | | |
| Study 1 | Lymphocytes | AVERAGE | Lymphocytes/WBC | RATIO | 0.001382 | 0.795972 | 0.4419753 |
| Study2 | Monocyte subset | CD40_CD14_CD20 | CD14pCD20nCD40pn | COUNT | 0.021 | 1 | 0.3695376 |
| Study2 | Monocyte subset | CD40_CD14_CD20 | CD14pCD20nCD40pn/ | RATIO | 0.003 | 1 | 0.6390621 |
| | | | CD14pCD20n | | | | |
| Study2 | Monocyte subset | HLADP_CD14_CD20 | CD14pCD20nDPp/ | COUNT | 0.009 | 1 | 0.5748591 |
| | | | CD14pCD20n | | | | |
| Study2 | Monocyte subset | HLADQ_CD14_CD20 | CD14pCD20nDQp | COUNT | 0.028 | 1 | 0.3862537 |
| Study2 | Monocyte subset | HLADQ_CD14_CD20 | CD14pCD20nDQp/ | RATIO | 0.003 | 1 | 0.6767177 |
| | | | CD14pCD20n | | | | |
| Study2 | Monocyte subset | HLADR_CD14_CD20 | CD14pCD20nDRp/ | RATIO | 0.003 | 1 | 0.5615718 |
| | | | CD14pCD20n | | | | |
| Study2 | Monocyte subset | HLAPAN_CD14_CD20 | CD14pCD20nPANp/ | RATIO | 0.017 | 1 | 0.5974805 |
| | | | CD14pCD20n | | | | |
| Study2 | Monocyte subset | CD54_CD14_CD3 | CD3nCD14pCD54p/ | COUNT | 0.02 | 1 | 0.5385682 |
| | | | CD3nCD14p | | | | |
| Study2 | Monocyte subset | CD101_CD14_CD4 | CD4pnCD14pCD101p/ | COUNT | 0.038 | 1 | 0.439986 |
| | | | CD4pnCD14p | | | | |
| Study2 | Monocyte subset | CD33_CD14_CD4 | CD4pnCD14pCD33p/ | COUNT | 0.017 | 1 | 0.3216624 |
| | | | CD4pnCD14p | | | | |
| Study2 | Monocyte subset | CD86_CD14_CD4 | CD4pnCD14pCD86p/ | COUNT | 0.035 | 1 | 0.5365573 |
| | | | CD4pnCD14p | | | | |
| Study 1 | Other | CCR5_CD60_CD14v9 | CCR5pCD14nCD60p | COUNT | 0.000043 | 0.025938 | 0.8062149 |
| Study 1 | Other | CCR5_CD60_CD4v8 | CCR5pCD4nCD60p | COUNT | 0.018091 | 1.000000 | 0.4447187 |
| Study 1 | Other | CD26_CD7_CD8v6 | CD7pCD8pnCD26n | COUNT | 0.005930 | 1.000000 | 0.2774447 |
| Study 1 | T Cell subset | CD158b_CD56_CD3v2 | CD3pCD158bp | COUNT | 0.043298 | 1.000000 | 0.1045154 |
| Study 1 | T Cell subset | CD161_CD56_CD3v2 | CD3pCD161p | COUNT | 0.001074 | 0.621719 | 0.5089205 |
| Study 1 | T cell subset | CD57_CD4_CD3v7 | CD3pCD4nCD57p | COUNT | 0.018212 | 1.000000 | 0.2056246 |
| Study 1 | T cell subset | CD94_CD56_CD3v2 | CD3pCD94p | COUNT | 0.019012 | 1.000000 | 0.2200139 |
| Study2 | T cell subset | TCRab_TCRgd_CD3 | CD3pTCRgdp | COUNT | 0.019 | 1 | 0.2370469 |
| Study 1 | T Cell subset | TCRab_TCRgd_CD3v8 | CD3pTCRgdp | COUNT | 0.000047 | 0.028634 | 0.3775772 |
| Study 1 | T Cell subset | CD7_CD6_CD4v7 | CD4nCD6pCD7p | COUNT | 0.000000 | 0.000261 | 0.8440452 |
| Study2 | T Cells | CD56_CD2_CD3 | CD2pCD3p | COUNT | 0.004 | 1 | 0.4343521 |
| Study2 | T Cells | NKB1_CD5_CD7 | CD5pCD7pNKB1n | COUNT | 0.016 | 1 | 0.2490271 |
| Study 1 | T cells | AVERAGE | T cells/WBC | RATIO | 0.001407 | 0.808844 | 0.6047076 |

1. An isolated marker for rheumatoid arthritis selected from the group consisting of
a) a marker selected from the group consisting of the markers set forth in Tables 1-8.
b) a polypeptide comprising an amino acid sequence selected from the group consisting of a polypeptide set forth in Tables 1-4;
c) a polypeptide comprising a homolog of a polypeptide of b), wherein said homolog shares 70% homology with the polypeptide of b) comprises a polypeptide;
d) a fragment of a polypeptide of b) or c);
e) a polynucleotide encoding any of the polypeptides of b), c), or d);
f) a polynucleotide encoding a homolog of a polypeptide of encoded by a nucleic acid sequence of e), and
g) a polypeptide which is fully complementary to a nucleic acid molecule of f).

2. A method for diagnosing rheumatoid arthritis in a subject, the method comprising:
a) obtaining a biological sample from the subject;
b) determining the level of a marker in the sample; and
c) comparing the level of the marker in the sample to a standard level or reference range.

3. The method of claim 2, wherein the marker is a marker of claim 1.

4. The method of claim 2, wherein the biological sample is a body fluid.

5. The method of claim 4, wherein the body fluid is selected from the group consisting of blood, serum, plasma, synovial fluid, urine, and saliva.

6. The method of claim 2, wherein the standard level or reference range is the level or range of the marker in at least one sample from a non-RA subject.

7. The method of claim 3, wherein the marker is not expressed in non-RA subjects.

8. The method of claim 3, wherein the level of the marker is determined by detecting the presence of a polypeptide.

9. The method of claim 8, wherein the polypeptide is the marker.

10. The method of claim 8, wherein the polypeptide is a modified form of the marker.

11. The method of claim 8, wherein the polypeptide is a precursor to the marker.

12. The method of claim 8, wherein the method further comprises detecting the presence of the polypeptide using a reagent that specifically binds to the polypeptide or a fragment thereof.

13. The method of claim 12, wherein the reagent is selected from the group consisting of an antibody, an antibody derivative, and an antibody fragment.

14. The method of claim 3, wherein the subject is a lab animal.

15. The method of claim 3, wherein the subject is a human subject.

16. A method for diagnosing rheumatoid arthritis in a subject, the method comprising:
a) obtaining one or more biological samples from the subject;
b) determining the level of a plurality of markers in the one or more biological samples, wherein at least one of the plurality of markers is a marker of claim 1; and
c) comparing the level of at least one of the plurality of markers to a reference value.

17. The method of claim 16, wherein at least one of the plurality of markers is a marker as set forth in Tables 1-8.

18. The method of claim 16, wherein the biological sample is a body fluid.

19. The method of claim 18, wherein the body fluid is selected from the group consisting of blood, serum, plasma, synovial fluid, urine, and saliva.

20. The method of claim 16, wherein at least two of the plurality of markers are a marker of claim 1

21. The method of claim 20, wherein at least two of the plurality of markers are selected from the group consisting of the markers set forth in Tables 1-8.

22. The method of claim 16, wherein at least ten of the plurality of markers are a marker of claim 1.

23. The method of claim 22, wherein at least ten of the plurality of markers are selected from the group consisting of the markers set forth in Tables 1-8.

24. The method of claim 16, wherein the standard level or reference range is the level of at least one of the plurality of markers in at least one sample from a non-RA subject, and wherein the level of the at least one of the plurality of markers is increased by at least one fold with respect to the reference value.

25. The method of claim 24, wherein the level of the at least one of the plurality of markers is increased by at least two fold with respect to the standard level or reference range.

26. The method of claim 16, wherein at least one of the plurality of markers is selected from the group consisting of the markers set forth in Tables 5-8.

27. The method of claim 26, wherein the reference value is the level of the at least one of the plurality of markers in at least one sample from a non-RA subject, and wherein the level of the at least one of the plurality of markers is increased by at least one fold with respect to the reference value.

28. The method of claim 27, wherein the level of the at least one of the plurality of markers is increased by at least two fold with respect to the reference value.

29. The method of claim 16, wherein the level of the at least two of the plurality of markers is indicative of differential expression in RA.

30. A method for monitoring the progression of rheumatoid arthritis in a subject, the method comprising:
a) obtaining a first biological sample from the subject;
b) measuring the level of a marker in the first sample, wherein the marker is a marker of claim 1;
c) obtaining a second biological sample from the subject;
d) measuring the level of the marker in the second sample; and
e) comparing the level of the marker measured in the first sample with the level of the marker measured in the second sample.

31. The method of claim 30, wherein said obtaining a first biological sample from the subject occurs a time t₀, and said obtaining a second biological sample from the subject occurs at a later time t₁.

32. The method of claim 31, wherein said obtaining a first biological sample from the said obtaining a second biological sample from the subject is repeated over a range of times.

33. The method of claim 30, wherein the marker is selected from the group consisting of the markers set forth in Tables 1-8.

34. The method of claim 30, wherein the marker is selected from the group consisting of the markers set forth in Tables 5-8.

35. A method of assessing the efficacy of a treatment for rheumatoid arthritis in a subject, the method comprising comparing:
i) the level of a marker measured in a first sample obtained from the subject at a time t₀, wherein the marker is selected from the group consisting of
a) a polypeptide comprising an amino acid sequence selected from the group consisting of a polypeptide set forth in Tables 1-4;
b) a polypeptide comprising a homolog of a polypeptide of a), wherein said homolog shares 70% homology with the polypeptide of a) comprises a polypeptide;
c) a fragment of a polypeptide of a) or b); and
d) a polynucleotide encoding any of the polypeptides of a), b), or c).
e) a polynucleotide encoding a homolog of a polypeptide of encoded by a nucleic acid sequence of (d), and
f) a polypeptide which is fully complementary to a nucleic acid molecule of (e); and
(ii) the level of the marker in a second sample obtained from the subject at time t₁,
wherein a decrease in the level of the marker in the second sample relative to the first sample is an indication that the treatment is efficacious for treating rheumatoid arthritis in the subject.

36. The method of claim 35, wherein said time t₀ is before the treatment has been administered to the subject, and said time t₁ is after the treatment has been administered to the subject.

37. The method of claim 36, wherein said comparing is repeated over a range of times.

38. A method of assessing the efficacy of a treatment for rheumatoid arthritis in a subject, the method comprising comparing:
(i) the level of a marker in a first sample obtained from the subject at a time t₀, wherein the marker is selected from the group consisting of the markers set forth in Tables 5-8; and
(ii) the level of the marker in a second sample obtained from the subject at a time t₁,
wherein an increase in the amount of the marker in the second sample, relative to the first sample, is an indication that the treatment is efficacious for inhibiting rheumatoid arthritis in the subject.

39. The method of claim 38, wherein said time t₀ is before the treatment has been administered to the subject, and said time t₁ is after the treatment has been administered to the subject.

40. The method of claim 39, wherein said comparing is repeated over a range of times.

41. A method of treating rheumatoid arthritis in a subject, the method comprising inhibiting expression of a gene corresponding to a polynucleotide marker selected from the group consisting of the markers set forth in Tables 1-4.

42. The method of claim 41, wherein the first marker is a molecule selected from the group consisting of the markers set forth in Tables 1-4.

43. The method of claim 41, wherein the second marker is a molecule selected from the group consisting of the markers set forth in Tables 5-8.

44. A composition comprising a molecule selected from the group selected from the group consisting of
a) a marker selected from the group consisting of the markers set forth in Tables 1-8.
b) a polypeptide comprising an amino acid sequence selected from the group consisting of a polypeptide set forth in Tables 1-4;
c) a polypeptide comprising a homolog of a polypeptide of b), wherein said homolog shares 70% homology with the polypeptide of b) comprises a polypeptide;
d) a fragment of a polypeptide of b) or c);
e) a polynucleotide encoding any of the polypeptides of b), c), or d);
f) a polynucleotide encoding a homolog of a polypeptide of encoded by a nucleic acid sequence of e), and
g) a polypeptide which is fully complementary to a nucleic acid molecule of f).

45. A method for determining the type, stage or severity of rheumatoid arthritis in a subject, the method comprising:
obtaining a biological sample from the subject;
determining the level of a marker in the sample, wherein the marker is a marker of claim 1;
comparing the level of the marker in the sample to a reference value; and
determining from the results of the comparison the type, stage or severity of Rheumatoid arthritis in the subject.

46. The method of claim 45, wherein the marker is selected from the group consisting of the markers set forth in Tables 1-8.

47. A method for determining the risk of developing rheumatoid arthritis in a subject, the method comprising:
obtaining a biological sample from the subject;
determining the level of a marker in the sample, wherein the marker is a marker of claim 1;
comparing the level of the marker in the sample to a reference value; and
determining from the results of the comparison that the subject has an increased or decreased risk of developing rheumatoid arthritis.

48. The method of claim 47, wherein the marker is selected from the group consisting of the markers set forth in Tables 1-8.

49. A kit comprising a marker selected from a marker of claim 2.

50. The kit of claim 49, wherein the marker is selected from the group consisting of the markers set forth in Tables 1-8.

51. A kit comprising a reagent that specifically binds to a marker of claim 2.

52. The kit of claim 51, wherein the marker is selected from the group consisting of the markers set forth in Tables 1-8.


SEQUENCE LISTING (395 sequences)

SEQ ID NO: 1 - protein, 15 aa, Homo sapiens
IECVSAETTEDCIAK

SEQ ID NO: 2 - protein, 17 aa, Homo sapiens
LAPNNLKPVVAEFYGSK

SEQ ID NO: 3 - protein, 20 aa, Homo sapiens
DAYLAPNNLKPVVAEFYGSK

SEQ ID NO: 4 - protein, 9 aa, Homo sapiens
LYCDLPEPR

SEQ ID NO: 5 - protein, 8 aa, Homo sapiens
YYAVAVVK

SEQ ID NO: 6 - protein, 12 aa, Homo sapiens
SAGWNIPIGLLY

SEQ ID NO: 7 - protein, 17 aa, Homo sapiens
EDLIWELLNQAQEHFGK

SEQ ID NO: 8 - protein, 13 aa, Homo sapiens
KPVEEYANCHLAR

SEQ ID NO: 9 - protein, 9 aa, Homo sapiens
LKCDEWSVN

SEQ ID NO: 10 - protein, 7 aa, Homo sapiens
SKEFQLF

SEQ ID NO: 11 - protein, 15 aa, Homo sapiens
ADRDQYELLCLDNTR

SEQ ID NO: 12 - protein, 12 aa, Homo sapiens
SLDGGFVYIAGK

SEQ ID NO: 13 - protein, 12 aa, Homo sapiens
DQYELLCLDNTR

SEQ ID NO: 14 - protein, 26 aa, Homo sapiens
LCMGSGLNLCEPNNKEGYYGYTGAFR

SEQ ID NO: 15 - protein, 11 aa, Homo sapiens
DYELLCLDGTR

SEQ ID NO: 16 - protein, 11 aa, Homo sapiens
HSTIFENLANK

SEQ ID NO: 17 - protein, 11 aa, Homo sapiens
CDEWSVNSVGK

SEQ ID NO: 18 - protein, 11 aa, Homo sapiens
KDSGFQMNQLR

SEQ ID NO: 19 - protein, 16 aa, Homo sapiens
EGTCPEAPTDECKPVK

SEQ ID NO: 20 - protein, 15 aa, Homo sapiens
DCHLAQVPSHTVVAR

SEQ ID NO: 21 - protein, 7 aa, Homo sapiens
NPDPWAK

SEQ ID NO: 22 - protein, 13 aa, Homo sapiens
CSTSSLLEACTFR

SEQ ID NO: 23 - protein, 16 aa, Homo sapiens
NLNEKDYELLCLDGTR

SEQ ID NO: 24 - protein, 19 aa, Homo sapiens
SDNCEDTPEAGYFAVAVVK

SEQ ID NO: 25 - protein, 8 aa, Homo sapiens
DDTVCLAK

SEQ ID NO: 26 - protein, 15 aa, Homo sapiens
  misc_feature (9)..(9): carboxymethylation
FDEFFSEGCAPGSKK

SEQ ID NO: 27 - protein, 10 aa, Homo sapiens
DSGFQMNQLR

SEQ ID NO: 28 - protein, 14 aa, Homo sapiens
TAGWNIPMGLLYNK

SEQ ID NO: 29 - protein, 8 aa, Homo sapiens
SCHTAVGR

SEQ ID NO: 30 - protein, 9 aa, Homo sapiens
WCALSHHER

SEQ ID NO: 31 - protein, 12 aa, Homo sapiens
MYLGYEYVTAIR

SEQ ID NO: 32 - protein, 6 aa, Homo sapiens
AVGNLR

SEQ ID NO: 33 - protein, 13 aa, Homo sapiens
LKCDEWSVNSVGK

SEQ ID NO: 34 - protein, 14 aa, Homo sapiens
EDPQTFYYAVAVVK

SEQ ID NO: 35 - protein, 10 aa, Homo sapiens
DGAGDVAFVK

SEQ ID NO: 36 - protein, 11 aa, Homo sapiens
WCAVSEHEATK

SEQ ID NO: 37 - protein, 11 aa, Homo sapiens
EGYYGYTGAFR

SEQ ID NO: 38 - protein, 14 aa, Homo sapiens
SVIPSDGPSVACVK

SEQ ID NO: 39 - protein, 10 aa, Homo sapiens
LGMFNIQHCK

SEQ ID NO: 40 - protein, 8 aa, Homo sapiens
GSFPWQAK

SEQ ID NO: 41 - protein, 13 aa, Homo sapiens
TEGDGVYTLNNEK

SEQ ID NO: 42 - protein, 11 aa, Homo sapiens
DIFTGLIGPMK

SEQ ID NO: 43 - protein, 23 aa, Homo sapiens
HYYIAAEEIIWNYAPSGIDIFTK

SEQ ID NO: 44 - protein, 10 aa, Homo sapiens
FTVLQDVPVR

SEQ ID NO: 45 - protein, 9 aa, Homo sapiens
TVLQDVPVR

SEQ ID NO: 46 - protein, 17 aa, Homo sapiens
MCPQLQQYEMHGPEGLR

SEQ ID NO: 47 - protein, 25 aa, Homo sapiens
VLLAYLTAQPAPTSEDLTSATNIVK

SEQ ID NO: 48 - protein, 14 aa, Homo sapiens
AVLPTGDVIGDSAK

SEQ ID NO: 49 - protein, 13 aa, Homo sapiens
LVHVEEPHTETVR

SEQ ID NO: 50 - protein, 10 aa, Homo sapiens
  MISC_FEATURE (50)..(50): carboxymethylation
YDVENCLANK

SEQ ID NO: 51 - protein, 11 aa, Homo sapiens
SSGSLLNNAIK

SEQ ID NO: 52 - protein, 13 aa, Homo sapiens
DNSVHWERPQKPK

SEQ ID NO: 53 - protein, 21 aa, Homo sapiens
VYDYYETDEFAIAEYNAPCSK

SEQ ID NO: 54 - protein, 18 aa, Homo sapiens
VSVQLEASPAFLAVPVEK

SEQ ID NO: 55 - protein, 23 aa, Homo sapiens
SVSGKPQYMVLVPSLLHTETTEK

SEQ ID NO: 56 - protein, 18 aa, Homo sapiens
  MISC_FEATURE (7)..(7): carboxymethylation
YSDASDCHGEDSQAFCEK

SEQ ID NO: 57 - protein, 19 aa, Homo sapiens
KYSDASDCHGEDSQAFCEK

SEQ ID NO: 58 - protein, 7 aa, Homo sapiens
GPTQEFK

SEQ ID NO: 59 - protein, 10 aa, Homo sapiens
QTVSWAVTPK

SEQ ID NO: 60 - protein, 14 aa, Homo sapiens
TAQEGDHGSHVYTK

SEQ ID NO: 61 - protein, 10 aa, Homo sapiens
YDVENCLANK

SEQ ID NO: 62 - protein, 14 aa, Homo sapiens
TEHPFTVEEFVLPK

SEQ ID NO: 63 - protein, 16 aa, Homo sapiens
QFSFPLSSEPFQGSYK

SEQ ID NO: 64 - protein, 11 aa, Homo sapiens
RHPYFYAPELL

SEQ ID NO: 65 - protein, 11 aa, Homo sapiens
FYAPELLFFAK

SEQ ID NO: 66 - protein, 16 aa, Homo sapiens
RPCFSALEVDETYVPK

SEQ ID NO: 67 - protein, 8 aa, Homo sapiens
TYETTLEK

SEQ ID NO: 68 - protein, 17 aa, Homo sapiens
VHTECCHGDLLECADDR

SEQ ID NO: 69 - protein, 14 aa, Homo sapiens
VPQVSTPTLVEVSR

SEQ ID NO: 70 - protein, 17 aa, Homo sapiens
SLHTLFGDKLCTVATLR

SEQ ID NO: 71 - protein, 12 aa, Homo sapiens
AAFTECCQAADK

SEQ ID NO: 72 - protein, 8 aa, Homo sapiens
DLGEENFK

SEQ ID NO: 73 - protein, 22 aa, Homo sapiens
LVRPEVDVMCTAFHDNEETFLK

SEQ ID NO: 74 - protein, 12 aa, Homo sapiens
RHPDYSVVLLLR

SEQ ID NO: 75 - protein, 9 aa, Homo sapiens
CCTESLVNR

SEQ ID NO: 76 - protein, 15 aa, Homo sapiens
KVPQVSTPTLVEVSR

SEQ ID NO: 77 - protein, 13 aa, Homo sapiens
ADDKETCFAEEGK

SEQ ID NO: 78 - protein, 17 aa, Homo sapiens
VFDEFKPLVEEPQNLIK

SEQ ID NO: 79 - protein, 5 aa, Homo sapiens
LDELR

SEQ ID NO: 80 - protein, 6 aa, Homo sapiens
AWAVAR

SEQ ID NO: 81 - protein, 12 aa, Homo sapiens
AVMDDFAAFVEK

SEQ ID NO: 82 - protein, 10 aa, Homo sapiens
LVNEVTEFAK

SEQ ID NO: 83 - protein, 14 aa, Homo sapiens
  MISC_FEATURE (8)..(9): carboxymethylation
YKAAFTECCQAADK

SEQ ID NO: 84 - protein, 12 aa, Homo sapiens
YICENQDSISSK

SEQ ID NO: 85 - protein, 12 aa, Homo sapiens
RHPYFYAPELLF

SEQ ID NO: 86 - protein, 10 aa, Homo sapiens
TEVNVLPGAK

SEQ ID NO: 87 - protein, 7 aa, Homo sapiens
TILDDLR

SEQ ID NO: 88 - protein, 11 aa, Homo sapiens
LSNENHGIAQR

SEQ ID NO: 89 - protein, 8 aa, Homo sapiens
VQSTITSR

SEQ ID NO: 90 - protein, 13 aa, Homo sapiens
AHVSFKPTVAQQR

SEQ ID NO: 91 - protein, 15 aa, Homo sapiens
IQPSGGTNINEALLR

SEQ ID NO: 92 - protein, 7 aa, Homo sapiens
IYLQPGR

SEQ ID NO: 93 - protein, 19 aa, Homo sapiens
VVNNSPQPQNVVFDVQIPK

SEQ ID NO: 94 - protein, 10 aa, Homo sapiens
SSALDMENFR

SEQ ID NO: 95 - protein, 7 aa, Homo sapiens
ALYAQAR

SEQ ID NO: 96 - protein, 15 aa, Homo sapiens
AEDHFSVIDFNQNIR

SEQ ID NO: 97 - protein, 9 aa, Homo sapiens
WFYIASAFR

SEQ ID NO: 98 - protein, 22 aa, Homo sapiens
GFYPSDIAVEWESNGQPENNYK

SEQ ID NO: 99 - protein, 12 aa, Homo sapiens
VVAGVANALAHK

SEQ ID NO: 100 - protein, 10 aa, Homo sapiens
LLVVYPWTQR

SEQ ID NO: 101 - protein, 13 aa, Homo sapiens
VNVDEVGGEALGR

SEQ ID NO: 102 - protein, 22 aa, Homo sapiens
  MISC_FEATURE: carboxymethylation
GTFATLSELHCDKLHVDPENFR

SEQ ID NO: 103 - protein, 19 aa, Homo sapiens
FFESFGDLSTPDAVMGNPK

SEQ ID NO: 104 - protein, 12 aa, Homo sapiens
EFTPPVQAAYQK

SEQ ID NO: 105 - protein, 13 aa, Homo sapiens
GTFATLSELHCDK

SEQ ID NO: 106 - protein, 6 aa, Homo sapiens
TLDPER

SEQ ID NO: 107 - protein, 8 aa, Homo sapiens
QLANGVDR

SEQ ID NO: 108 - protein, 11 aa, Homo sapiens
  misc_feature (8)..(8): carboxymethylation
DIPMNPMCIYR

SEQ ID NO: 109 - protein, 13 aa, Homo sapiens
FATTFYQHLADSK

SEQ ID NO: 110 - protein, 11 aa, Homo sapiens
DDLYVSDAFHK

SEQ ID NO: 111 - protein, 7 aa, Homo sapiens
LQPLDFK

SEQ ID NO: 112 - protein, 16 aa, Homo sapiens
EQLQDMGLVDLFSPEK

SEQ ID NO: 113 - protein, 23 aa, Homo sapiens
ALGISPFHEHAEVVFTANDSGPR

SEQ ID NO: 114 - protein, 13 aa, Homo sapiens
AADDTWEPFASGK

SEQ ID NO: 115 - protein, 13 aa, Homo sapiens
GSPAINVAVHVFR

SEQ ID NO: 116 - protein, 22 aa, Homo sapiens
TSESGELHGLTTEEEFVEGIYK

SEQ ID NO: 117 - protein, 29 aa, Homo sapiens
FVSEAGPTGTGESKCPLMVKVLDAVRGSP

SEQ ID NO: 118 - protein, 12 aa, Homo sapiens
IYGNQDTSSQLK

SEQ ID NO: 119 - protein, 22 aa, Homo sapiens
  misc_feature (12)..(13): carboxymethylation
MLADAPPQDPSCCSGALYYGSK

SEQ ID NO: 120 - protein, 13 aa, Homo sapiens
IYGNQDTSSQLKK

SEQ ID NO: 121 - protein, 8 aa, Homo sapiens
HADPDFTR

SEQ ID NO: 122 - protein, 19 aa, Homo sapiens
  misc_feature (18)..(18): carboxymethylation
MHSMNGFMYGNQPGLTMCK

SEQ ID NO: 123 - protein, 12 aa, Homo sapiens
FEDGVLDPDYPR

SEQ ID NO: 124 - protein, 10 aa, Homo sapiens
  misc_feature (4)..(4): carboxymethylation
GQYCYELDEK

SEQ ID NO: 125 - protein, 6 aa, Homo sapiens
ANLFNK

SEQ ID NO: 126 - protein, 8 aa, Homo sapiens
YTACETAR

SEQ ID NO: 127 - protein, 20 aa, Homo sapiens
  misc_feature (6)..(6): carboxymethylation
DKLAACLEGNCAEGLGTNYR

SEQ ID NO: 128 - protein, 15 aa, Homo sapiens
VGAHAGEYGAEALER

SEQ ID NO: 129 - protein, 30 aa, Homo sapiens
KVADALTNAVAHVDDMPNALSALSDLHAHK

SEQ ID NO: 130 - protein, 29 aa, Homo sapiens
VADALTNAVAHVDDMPNALSALSDLHAHK

SEQ ID NO: 131 - protein, 14 aa, Homo sapiens
LLDNWDSVTSTFSK

SEQ ID NO: 132 - protein, 11 aa, Homo sapiens
QGLLPVLESFK

SEQ ID NO: 133 - protein, 20 aa, Homo sapiens
QSNNKYAASSYLSLTPEQWK

SEQ ID NO: 134 - protein, 19 aa, Homo sapiens
LQQVLHAGSGPCLPHLLSR

SEQ ID NO: 135 - protein, 9 aa, Homo sapiens
QVEGMEDWK

SEQ ID NO: 136 - protein, 17 aa, Homo sapiens
EQLGEFYEALDCLCIPR

SEQ ID NO: 137 - protein, 7 aa, Homo sapiens
MLSLGTK

SEQ ID NO: 138 - protein, 7 aa, Homo sapiens
ATGIPDR

SEQ ID NO: 139 - protein, 15 aa, Homo sapiens
EEEQQRCESLAEVNT

SEQ ID NO: 140 - protein, 12 aa, Homo sapiens
MNQLTQELFSLK

SEQ ID NO: 141 - protein, 8 aa, Homo sapiens
VTSTLTIK

SEQ ID NO: 142 - protein, 13 aa, Homo sapiens
NVPLPVIAELPPK

SEQ ID NO: 143 - protein, 16 aa, Homo sapiens
VVSVLTVVHQDWLNGK

SEQ ID NO: 144 - protein, 17 aa, Homo sapiens
TTPPMLDSDGSFFLYSK

SEQ ID NO: 145 - protein, 10 aa, Homo sapiens
LPECEAVCGK

SEQ ID NO: 146 - protein, 19 aa, Homo sapiens
RLYGSEAFATDFQDSAAAK

SEQ ID NO: 147 - protein, 14 aa, Homo sapiens
EHAVEGDCDFQLLK

SEQ ID NO: 148 - protein, 20 aa, Homo sapiens
  misc_feature (5)..(5): carboxymethylation
KEDSCQLGYSAGPCMGMTSR

SEQ ID NO: 149 - protein, 8 aa, Homo sapiens
EQLTPLIK

SEQ ID NO: 150 - protein, 10 aa, Homo sapiens
EQHPDMSVTR

SEQ ID NO: 151 - protein, 13 aa, Homo sapiens
AGALNSNDAFVLK

SEQ ID NO: 152 - protein, 19 aa, Homo sapiens
GSLVQASEANLQAAQDFVR

SEQ ID NO: 153 - protein, 14 aa, Homo sapiens
  misc_feature (8)..(8): carboxymethylation
IASFSQNCDIYPGK

SEQ ID NO: 154 - protein, 33 aa, Homo sapiens
CGLVPVLAENYKSQQSSDPDPNCVDRPVEGYLA

SEQ ID NO: 155 - protein, 10 aa, Homo sapiens
ISNIPDEYFK

SEQ ID NO: 156 - protein, 11 aa, Homo sapiens
SLEDLQLTHNK

SEQ ID NO: 157 - protein, 9 aa, Homo sapiens
QNGGLATVE

SEQ ID NO: 158 - protein, 15 aa, Homo sapiens
  misc_feature (1)..(1): oxidation
MCEQALGKGCGGDSK

SEQ ID NO: 159 - protein, 18 aa, Homo sapiens
  misc_feature (8)..(8): carboxymethylation
LLNLDGTCADSYSFVFSR

SEQ ID NO: 160 - protein, 10 aa, Homo sapiens
AGKSTFLKKH

SEQ ID NO: 161 - protein, 18 aa, Homo sapiens
EIVMTQSPATLSVSPGER

SEQ ID NO: 162 - protein, 12 aa, Homo sapiens
EGLCCGPSIPPV

SEQ ID NO: 163 - protein, 8 aa, Homo sapiens
AAYMNKER

SEQ ID NO: 164 - protein, 11 aa, Homo sapiens
YYCFQGNQFLR

SEQ ID NO: 165 - protein, 14 aa, Homo sapiens
  misc_feature (7)..(7): carboxymethylation
GGCLPPCDGGPKSR

SEQ ID NO: 166 - protein, 12 aa, Homo sapiens
ASDDDVGENARI

SEQ ID NO: 167 - protein, 9 aa, Homo sapiens
EEAIAVTMR

SEQ ID NO: 168 - protein, 14 aa, Homo sapiens
YNPDSGLEVLAVQR

SEQ ID NO: 169 - protein, 10 aa, Homo sapiens
IVDLVKELDR

SEQ ID NO: 170 - protein, 18 aa, Homo sapiens
HKLIHTGVKSHACEQCGK

SEQ ID NO: 171 - protein, 18 aa, Homo sapiens
VFWRSSGLPHPSQAQSAR

SEQ ID NO: 172 - protein, 16 aa, Homo sapiens
  misc_feature (7)..(7): carboxymethylation
GNALSVCSRESPGSKK

SEQ ID NO: 173 - protein, 36 aa, Homo sapiens
CLQRIVTKLQMEAGLCEEQLNQADALLQSDVRLLAA

SEQ ID NO: 174 - protein, 20 aa, Homo sapiens
IITHPNFNGNTLDNDIMLIK

SEQ ID NO: 175 - protein, 15 aa, Homo sapiens
FTVDRPFLFLIYEHR

SEQ ID NO: 176 - protein, 11 aa, Homo sapiens
GGSIFGLAPGK

SEQ ID NO: 177 - protein, 9 aa, Homo sapiens
GQGKPPVWR

SEQ ID NO: 178 - protein, 31 aa, Homo sapiens
AVGDKLPECEADDGCPKPPEIAHGYVEHSVR

SEQ ID NO: 179 - protein, 10 aa, Homo sapiens
FSEGCAPGSK

SEQ ID NO: 180 - protein, 15 aa, Homo sapiens
LCMGSGLNLCEPNNK

SEQ ID NO: 181 - protein, 20 aa, Homo sapiens
  misc_feature (4)..(4): carboxymethylation
SDNCEDTPEAGYFAVAVVKK

SEQ ID NO: 182 - protein, 8 aa, Homo sapiens
YLGEEYVK

SEQ ID NO: 183 - protein, 22 aa, Homo sapiens
SMGGKEDLIWELLNQAQEHFGK

SEQ ID NO: 184 - protein, 15 aa, Homo sapiens
LCMGSGLNLCEPNNK

SEQ ID NO: 185 - protein, 6 aa, Homo sapiens
DSSLCK

SEQ ID NO: 186 - protein, 8 aa, Homo sapiens
QINDYVEK

SEQ ID NO: 187 - protein, 6 aa, Homo sapiens
FLEDVK

SEQ ID NO: 188 - protein, 14 aa, Homo sapiens
ITPNLAEFAFSLYR

SEQ ID NO: 189 - protein, 18 aa, Homo sapiens
LYHSEAFTVNFGDTEEAK

SEQ ID NO: 190 - protein, 18 aa, Homo sapiens
VFSNGADLSGVTEEAPLK

SEQ ID NO: 191 - protein, 9 aa, Homo sapiens
KQINDYVEK

SEQ ID NO: 192 - protein, 10 aa, Homo sapiens
SVLGQLGITK

SEQ ID NO: 193 - protein, 9 aa, Homo sapiens
KQINDYVEK

SEQ ID NO: 194 - protein, 15 aa, Homo sapiens
TDTSHHDQDHPTFNK

SEQ ID NO: 195 - protein, 7 aa, Homo sapiens
SPLFMGK

SEQ ID NO: 196 - protein, 8 aa, Homo sapiens
SASLHLPK

SEQ ID NO: 197 - protein, 8 aa, Homo sapiens
SASLHLPK

SEQ ID NO: 198 - protein, 8 aa, Homo sapiens
WERPFEVK

SEQ ID NO: 199 - protein, 8 aa, Homo sapiens
AVLTIDEK

SEQ ID NO: 200 - protein, 10 aa, Homo sapiens
EDPQGDAAQK

SEQ ID NO: 201 - protein, 22 aa, Homo sapiens
GTEAAGAMFLEAIPMSIPPEVK

SEQ ID NO: 202 - protein, 16 aa, Homo sapiens
DTEEEDFHVDQVTTVK

SEQ ID NO: 203 - protein, 8 aa, Homo sapiens
QINDYVEK

SEQ ID NO: 204 - protein, 8 aa, Homo sapiens
FLENEDRR

SEQ ID NO: 205 - protein, 10 aa, Homo sapiens
KLSSWVLLMK

SEQ ID NO: 206 - protein, 24 aa, Homo sapiens
TLNQPDSQLQLTTGNGLFLSEGLK

SEQ ID NO: 207 - protein, 11 aa, Homo sapiens
LVDKFLEDVKK

SEQ ID NO: 208 - protein, 5 aa, Homo sapiens
VPMMK

SEQ ID NO: 209 - protein, 17 aa, Homo sapiens
ELDRDTVFALVNYIFFK

SEQ ID NO: 210 - protein, 19 aa, Homo sapiens
LYHSEAFTVNFGDTEEAKK

SEQ ID NO: 211 - protein, 34 aa, Homo sapiens
MFNIQHCKKLSSWVLLMKYLGNATAIFFLPDEGK

SEQ ID NO: 212 - protein, 6 aa, Homo sapiens
VSVNER

SEQ ID NO: 213 - protein, 6 aa, Homo sapiens
KQWINK

SEQ ID NO: 214 - protein, 9 aa, Homo sapiens
HTFCAGMSK

SEQ ID NO: 215 - protein, 10 aa, Homo sapiens
HYEGSTVPEK

SEQ ID NO: 216 - protein, 12 aa, Homo sapiens
TEGDGVYTLNDK

SEQ ID NO: 217 - protein, 12 aa, Homo sapiens
DIAPTLTLYVGK

SEQ ID NO: 218 - protein, 34 aa, Homo sapiens
YQEDTCYGDAGSAFAVHDLEEDTWYATGILSFDK

SEQ ID NO: 219 - protein, 11 aa, Homo sapiens
HYEGSTVPEKK

SEQ ID NO: 220 - protein, 13 aa, Homo sapiens
TEGDGVYTLNDKK

SEQ ID NO: 221 - protein, 7 aa, Homo sapiens
QLVEIEK

SEQ ID NO: 222 - protein, 6 aa, Homo sapiens
VSVNER

SEQ ID NO: 223 - protein, 7 aa, Homo sapiens
NPANPVQ

SEQ ID NO: 224 - protein, 7 aa, Homo sapiens
DYAEVGR

SEQ ID NO: 225 - protein, 12 aa, Homo sapiens
SCAVAEYGVYVK

SEQ ID NO: 226 - protein, 20 aa, Homo sapiens
SPVGVQPILNEHTFCAGMSK

SEQ ID NO: 227 - protein, 10 aa, Homo sapiens
VTSIQDWVQK

SEQ ID NO: 228 - protein, 17 aa, Homo sapiens
AVGDKLPECEAVCGKPK

SEQ ID NO: 229 - protein, 9 aa, Homo sapiens
VGYVSGWGR

SEQ ID NO: 230 - protein, 13 aa, Homo sapiens
DIAPTLTLYVGKK

SEQ ID NO: 231 - protein, 9 aa, Homo sapiens
ILGGHLDAK

SEQ ID NO: 232 - protein, 4 aa, Homo sapiens
NYYK

SEQ ID NO: 233 - protein, 26 aa, Homo sapiens
LPECEADDGCPKPPEIAHGYVEHSVR

SEQ ID NO: 234 - protein, 31 aa, Homo sapiens
VDSGNDVTDIADDGCPKPPEIAHGYVEHSVR

SEQ ID NO: 235 - protein, 15 aa, Homo sapiens
LRTEGDGVYTLNDKK

SEQ ID NO: 236 - protein, 28 aa, Homo sapiens
QKDVDKEFYLFPTVFDENESLLLEDNIR

SEQ ID NO: 237 - protein, 20 aa, Homo sapiens
AEEEHLGILGPQLHADVGDK

SEQ ID NO: 238 - protein, 13 aa, Homo sapiens
VDKDNEDFQESNR

SEQ ID NO: 239 - protein, 10 aa, Homo sapiens
IYHSHIDAPK

SEQ ID NO: 240 - protein, 8 aa, Homo sapiens
TYCSEPEK

SEQ ID NO: 241 - protein, 14 aa, Homo sapiens
EVGPTNADPVCLAK

SEQ ID NO: 242 - protein, 14 aa, Homo sapiens
DIASGLIGPLIICK

SEQ ID NO: 243 - protein, 19 aa, Homo sapiens
LISVDTEHSNIYLQNGPDR

SEQ ID NO: 244 - protein, 35 aa, Homo sapiens
NMATRPYSIHAHGVQTESSTVTPTLPGETLTYVWK

SEQ ID NO: 245 - protein, 12 aa, Homo sapiens
ALYLQYTDETFR

SEQ ID NO: 246 - protein, 8 aa, Homo sapiens
QYTDSTFR

SEQ ID NO: 247 - protein, 10 aa, Homo sapiens
MYYSAVDPTK

SEQ ID NO: 248 - protein, 20 aa, Homo sapiens
HYYIGIIETTWDYASDHGEK

SEQ ID NO: 249 - protein, 23 aa, Homo sapiens
GPEEEHLGILGPVIWAEVGDTIR

SEQ ID NO: 250 - protein, 10 aa, Homo sapiens
EYTDASFTNR

SEQ ID NO: 251 - protein, 20 aa, Homo sapiens
KLISVDTEHSNIYLQNGPDR

SEQ ID NO: 252 - protein, 19 aa, Homo sapiens
MHSMNGFMYGNQPGLTMCK

SEQ ID NO: 253 - protein, 14 aa, Homo sapiens
DLYSGLIGPLIVCR

SEQ ID NO: 254 - protein, 13 aa, Homo sapiens
GAYPLSIEPIGVR

SEQ ID NO: 255 - protein, 5 aa, Homo sapiens
VFNPR

SEQ ID NO: 256 - protein, 21 aa, Homo sapiens
KAEEEHLGILGPQLHADVGDK

SEQ ID NO: 257 - protein, 13 aa, Homo sapiens
RQSEDSTFYLGER

SEQ ID NO: 258 - protein, 7 aa, Homo sapiens
YTVNQCR

SEQ ID NO: 259 - protein, 6 aa, Homo sapiens
VDSHFR

SEQ ID NO: 260 - protein, 13 aa, Homo sapiens
RHPYFYAPELLFF

SEQ ID NO: 261 - protein, 8 aa, Homo sapiens
NECFLQHK

SEQ ID NO: 262 - protein, 9 aa, Homo sapiens
QTALVELVK

SEQ ID NO: 263 - protein, 14 aa, Homo sapiens
VFDEFKPLVEEPQN

SEQ ID NO: 264 - protein, 10 aa, Homo sapiens
ADLSGITGAR

SEQ ID NO: 265 - protein, 19 aa, Homo sapiens
AVLDVFEEGTEASAATAVK

SEQ ID NO: 266 - protein, 15 aa, Homo sapiens
MEEVEAMLLPETLKR

SEQ ID NO: 267 - protein, 10 aa, Homo sapiens
NLAVSQVVHK

SEQ ID NO: 268 - protein, 10 aa, Homo sapiens
ADLSGITGAR

SEQ ID NO: 269 - protein, 11 aa, Homo sapiens
ITLLSALVETR

SEQ ID NO: 270 - protein, 12 aa, Homo sapiens
DEELSCTVVELK

SEQ ID NO: 271 - protein, 22 aa, Homo sapiens
FNRPFLMIIVPTDTQNIFFMSK

SEQ ID NO: 272 - protein, 18 aa, Homo sapiens
LYGSEAFATDFQDSAAAK

SEQ ID NO: 273 - protein, 20 aa, Homo sapiens
DYNLNDILLQLGIEEAFTSK

SEQ ID NO: 274 - protein, 8 aa, Homo sapiens
KLINDYVK

SEQ ID NO: 275 - protein, 6 aa, Homo sapiens
DSLEFR

SEQ ID NO: 276 - protein, 23 aa, Homo sapiens
NALTGLPPGLFQASATLDTLVLK

SEQ ID NO: 277 - protein, 6 aa, Homo sapiens
DCQVFR

SEQ ID NO: 278 - protein, 9 aa, Homo sapiens
GQTLLAVAK

SEQ ID NO: 279 - protein, 7 aa, Homo sapiens
GPLQLER

SEQ ID NO: 280 - protein, 21 aa, Homo sapiens
DGFDISGNPWICDQNLSDLYR

SEQ ID NO: 281 - protein, 26 aa, Homo sapiens
LQELHLSSNGLESLSPEFLRPVPQLR

SEQ ID NO: 282 - protein, 18 aa, Homo sapiens
TLDLGENQLETLPPDLLR

SEQ ID NO: 283 - protein, 10 aa, Homo sapiens
DLLLPQPDLR

SEQ ID NO: 284 - protein, 11 aa, Homo sapiens
ALGHLDLSGNR

SEQ ID NO: 285 - protein, 8 aa, Homo sapiens
CAGPEAVK

SEQ ID NO: 286 - protein, 16 aa, Homo sapiens
ENQLEVLEVSWLHGLK

SEQ ID NO: 287 - protein, 14 aa, Homo sapiens
EQLGEFYEALDCLR

SEQ ID NO: 288 - protein, 9 aa, Homo sapiens
KDKCEPLEK

SEQ ID NO: 289 - protein, 6 aa, Homo sapiens
CEPLEK

SEQ ID NO: 290 - protein, 9 aa, Homo sapiens
KDKCEPLEK

SEQ ID NO: 291 - protein, 27 aa, Homo sapiens
TYMLAFDVNDEKNWGLSVYADKPETTK

SEQ ID NO: 292 - protein, 12 aa, Homo sapiens
TYMLAFDVNDEK

SEQ ID NO: 293 - protein, 8 aa, Homo sapiens
DKCEPLEK

SEQ ID NO: 294 - protein, 10 aa, Homo sapiens
SDVVYTDWKK

SEQ ID NO: 295 - protein, 29 aa, Homo sapiens
  misc_feature (27)..(27): carboxymethylation
NWGLSVYADKPETTKEQLGEFYEALDCLR

SEQ ID NO: 296 - protein, 7 aa, Homo sapiens
DTLMISR

SEQ ID NO: 297 - protein, 30 aa, Homo sapiens
SCDKTHTCPPCPAPELLGGPSVFLFPPKPK

SEQ ID NO: 298 - protein, 19 aa, Homo sapiens
TPEVTCVVVDVSHEDPEVK

SEQ ID NO: 299 - protein, 14 aa, Homo sapiens
STSGGTAALGCLVK

SEQ ID NO: 300 - protein, 14 aa, Homo sapiens
FNWYVDGVEVHNAK

SEQ ID NO: 301 - protein, 16 aa, Homo sapiens
SNLDEDIIAEENIVSR

SEQ ID NO: 302 - protein, 7 aa, Homo sapiens
VVPEGIR

SEQ ID NO: 303 - protein, 5 aa, Homo sapiens
FALVR

SEQ ID NO: 304 - protein, 9 aa, Homo sapiens
CLAPLEGAR

SEQ ID NO: 305 - protein, 15 aa, Homo sapiens
LELHVDGPPPRPQLR

SEQ ID NO: 306 - protein, 19 aa, Homo sapiens
IFFHLNAVALGDGGHYTCR

SEQ ID NO: 307 - protein, 6 aa, Homo sapiens
VEIDTK

SEQ ID NO: 308 - protein, 10 aa, Homo sapiens
DDEEFIESNK

SEQ ID NO: 309 - protein, 29 aa, Homo sapiens
VYPGEQYTYMLLATEEQSPGEGDGNCVTR

SEQ ID NO: 310 - protein, 21 aa, Homo sapiens
  misc_feature (3)..(3): carboxymethylation
TYCSEPEKVDKDNEDFQESNR

SEQ ID NO: 311 - protein, 13 aa, Homo sapiens
KALYLQYTDETFR

SEQ ID NO: 312 - protein, 16 aa, Homo sapiens
DWHGVPGQVDAAMAGR

SEQ ID NO: 313 - protein, 9 aa, Homo sapiens
IYISGMAPR

SEQ ID NO: 314 - protein, 7 aa, Homo sapiens
LAIPEGK

SEQ ID NO: 315 - protein, 10 aa, Homo sapiens
SPAFTDLHLR

SEQ ID NO: 316 - protein, 7 aa, Homo sapiens
VAAEDWK

SEQ ID NO: 317 - protein, 17 aa, Homo sapiens
QEPSQGTTTFAVTSILR

SEQ ID NO: 318 - protein, 10 aa, Homo sapiens
WLQGSQELPR

SEQ ID NO: 319 - protein, 17 aa, Homo sapiens
QEPSQGTTTFAVTSILR

SEQ ID NO: 320 - protein, 11 aa, Homo sapiens
  misc_feature (3)..(3): carboxymethylation
LICQATGFSPR

SEQ ID NO: 321 - protein, 25 aa, Homo sapiens
  misc_feature (24)..(24): carboxymethylation
YAATSQVLLPSKDVMQGTDEHVVCK

SEQ ID NO: 322 - protein, 13 aa, Homo sapiens
  misc_feature (5)..(5): carboxymethylation
SKLICQATGFSPR

SEQ ID NO: 323 - protein, 18 aa, Homo sapiens
SLGECCDVEDSTTCFNAK

SEQ ID NO: 324 - protein, 7 aa, Homo sapiens
VLEPTLK

SEQ ID NO: 325 - protein, 22 aa, Homo sapiens
VPTADLEDVLPLAEDITNILSK

SEQ ID NO: 326 - protein, 19 aa, Homo sapiens
LAVTTHGLPCLAWASAQAK

SEQ ID NO: 327 - protein, 9 aa, Homo sapiens
LSPLGEEMR

SEQ ID NO: 328 - protein, 20 aa, Homo sapiens
ATLVCLISDFYPGAVTVAWK

SEQ ID NO: 329 - protein, 8 aa, Homo sapiens
LTVLGQPK

SEQ ID NO: 330 - protein, 11 aa, Homo sapiens
LCQDLGPGAFR

SEQ ID NO: 331 - protein, 7 aa, Homo sapiens
FAFNLYR

SEQ ID NO: 332 - protein, 6 aa, Homo sapiens
EVLLPK

SEQ ID NO: 333 - protein, 12 aa, Homo sapiens
QKWEAEPVYVQR

SEQ ID NO: 334 - protein, 9 aa, Homo sapiens
SDVMYTDWK

SEQ ID NO: 335 - protein, 9 aa, Homo sapiens
EQINNYVEK

SEQ ID NO: 336 - protein, 20 aa, Homo sapiens
VDNALQSGNSQESVTEQDSK

SEQ ID NO: 337 - protein, 20 aa, Homo sapiens
VDNALQSGNSQESVTEQDSK

SEQ ID NO: 338 - protein, 6 aa, Homo sapiens
TQQRNN

SEQ ID NO: 339 - protein, 6 aa, Homo sapiens
LELSQR

SEQ ID NO: 340 - protein, 14 aa, Homo sapiens
LRTEGDGVYTLNDK

SEQ ID NO: 341 - protein, 8 aa, Homo sapiens
KQLVEIEK

SEQ ID NO: 342 - protein, 23 aa, Homo sapiens
SVPPSASHVAPTETFTYEWTVPK

SEQ ID NO: 343 - protein, 16 aa, Homo sapiens
NNEGTYYSPNYNPQSR

SEQ ID NO: 344 - protein, 15 aa, Homo sapiens
VFAIPPSFASIFLTK

SEQ ID NO: 345 - protein, 25 aa, Homo sapiens
HYTNPSQDVTVPCPVPPPPPCCHPR

SEQ ID NO: 346 - protein, 19 aa, Homo sapiens
VVSVLTVLHQNWLDGKEYK

SEQ ID NO: 347 - protein, 10 aa, Homo sapiens
DFTCVHQALK

SEQ ID NO: 348 - protein, 9 aa, Homo sapiens
LLIYGASSR

SEQ ID NO: 349 - protein, 11 aa, Homo sapiens
KAACLDILMLR

SEQ ID NO: 350 - protein, 15 aa, Homo sapiens
DVWGIEGPIDAAFTR

SEQ ID NO: 351 - protein, 7 aa, Homo sapiens
EDTNKWK

SEQ ID NO: 352 - protein, 19 aa, Homo sapiens
ANAGKPKDPTFIPAPIQAK

SEQ ID NO: 353 - protein, 9 aa, Homo sapiens
AVYDQSATA

SEQ ID NO: 354 - protein, 12 aa, Homo sapiens
DSSTWLTAFVLK

SEQ ID NO: 355 - protein, 10 aa, Homo sapiens
PMPVLLMGQA

SEQ ID NO: 356 - protein, 18 aa, Homo sapiens
SETEIHQGFQHLHQLFAK

SEQ ID NO: 357 - protein, 9 aa, Homo sapiens
KYFIDFVAR

SEQ ID NO: 358 - protein, 14 aa, Homo sapiens
  misc_feature (6)..(6): carboxymethylation
EEPIPCTAHWHFGQ

SEQ ID NO: 359 - protein, 13 aa, Homo sapiens
HNLKDAGEAEEGK

SEQ ID NO: 360 - protein, 13 aa, Homo sapiens
GLSRTSMKPRSSR

SEQ ID NO: 361 - protein, 7 aa, Homo sapiens
DSSYMPS

SEQ ID NO: 362 - protein, 5 aa, Homo sapiens
LPLIK

SEQ ID NO: 363 - protein, 10 aa, Homo sapiens
IAEFAFEYAR

SEQ ID NO: 364 - protein, 10 aa, Homo sapiens
EGKLENGYRK

SEQ ID NO: 365 - protein, 15 aa, Homo sapiens
PQLDLFSCMLKHRLK

SEQ ID NO: 366 - protein, 21 aa, Homo sapiens
EAPTSLSQLLDNSGAPNVTIK

SEQ ID NO: 367 - protein, 9 aa, Homo sapiens
KVNEKDVDK

SEQ ID NO: 368 - protein, 17 aa, Homo sapiens
  misc_feature (5)..(5): carboxymethylation
GQELCADYSENTFTEYK

SEQ ID NO: 369 - protein, 9 aa, Homo sapiens
KNGNVANYV

SEQ ID NO: 370 - protein, 12 aa, Homo sapiens
  misc_feature (1)..(1): oxidation
MPVINIEDLTEK

SEQ ID NO: 371 - protein, 10 aa, Homo sapiens
LGKSVVAKVK

SEQ ID NO: 372 - protein, 7 aa, Homo sapiens
  misc_feature (2)..(2): oxidation
IMKDVQK

SEQ ID NO: 373 - protein, 16 aa, Homo sapiens
  misc_feature (8)..(8): carboxymethylation
ANPGYKWCPTTNKPVK

SEQ ID NO: 374 - protein, 12 aa, Homo sapiens
LGDFGIRLLCVG

SEQ ID NO: 375 - protein, 16 aa, Homo sapiens
FDDQNLRSVNGAEITM

SEQ ID NO: 376 - protein, 10 aa, Homo sapiens
ELDSQLNEPR

SEQ ID NO: 377 - protein, 9 aa, Homo sapiens
KTTNQNVIK

SEQ ID NO: 378 - protein, 9 aa, Homo sapiens
LSSWVLLMK

SEQ ID NO: 379 - protein, 13 aa, Homo sapiens
TLVITSTPASPNR

SEQ ID NO: 380 - protein, 11 aa, Homo sapiens
KGAAKVMVTNV

SEQ ID NO: 381 - protein, 15 aa, Homo sapiens
  misc_feature (3)..(3): oxidation
TEMRNSENKNIFCVR

SEQ ID NO: 382 - protein, 12 aa, Homo sapiens
TQTVECTQTGSV

SEQ ID NO: 383 - protein, 12 aa, Homo sapiens
KMKEAAQRYQYA

SEQ ID NO: 384 - protein, 11 aa, Homo sapiens
PREEQFNSTFR

SEQ ID NO: 385 - protein, 27 aa, Homo sapiens
MGPGGGKAKALGGAGSGSKGSAGGGSK

SEQ ID NO: 386 - protein, 19 aa, Homo sapiens
TGNNRINITETGQLMVKDF

SEQ ID NO: 387 - protein, 7 aa, Homo sapiens
LELFMGK

SEQ ID NO: 388 - protein, 21 aa, Homo sapiens
ELGVDQESEEGKGKTSPDKQK

SEQ ID NO: 389 - protein, 8 aa, Homo sapiens
NANAVCDT

SEQ ID NO: 390 - protein, 8 aa, Homo sapiens
MPQVFNFL

SEQ ID NO: 391 - protein, 16 aa, Homo sapiens
IAPQLSTEELVSLGEK

SEQ ID NO: 392 - protein, 17 aa, Homo sapiens
ECGKAFYSGSSLTQHQR

SEQ ID NO: 393 - protein, 11 aa, Homo sapiens
FVPQDVPPEPK

SEQ ID NO: 394 - protein, 6 aa, Homo sapiens
LTLDEK

SEQ ID NO: 395 - protein, 18 aa, Homo sapiens
DIQMTQSPSSVSASVGDR